SEQUENCE LISTING (60 sequences)

SEQ ID NO: 1 - DNA, 22 bp, Artificial
  Synthetic, Forward primer Chr7
ataaaggttcctgtgatgtcag

SEQ ID NO: 2 - DNA, 27 bp, Artificial
  Synthetic, Reverse primer Chr7
gtaagttatttagatgattttgaatgc

SEQ ID NO: 3 - DNA, 27 bp, Artificial
  Synthetic, Forward primer Chr13
attaaacatgataaaagggattcttac

SEQ ID NO: 4 - DNA, 23 bp, Artificial
  Synthetic, Forward primer Chr13
ataatgagcacttagagcaggat

SEQ ID NO: 5 - DNA, 27 bp, Artificial
  Synthetic, Reverse primer Chr20
aaacgtgtgctcttttccccatcttat

SEQ ID NO: 6 - DNA, 26 bp, Artificial
  Synthetic, Reverse primer Chr20
gctagccctctctggattccttcttc

SEQ ID NO: 7 - DNA, 20 bp, Artificial
  Synthetic, Forward primer ALDH1A1
gggcagccatttcttctcac

SEQ ID NO: 8 - DNA, 20 bp, Artificial
  Synthetic, Reverse primer ALDH1A1
cttcttagcccgctcaacac

SEQ ID NO: 9 - DNA, 20 bp, Artificial
  Synthetic, Forward primer DAT1
agtgtgcccatgagtaagag

SEQ ID NO: 10 - DNA, 20 bp, Artificial
  Synthetic, Reverse primer DAT1
tcccagcaatgaccatgaag

SEQ ID NO: 11 - DNA, 20 bp, Artificial
  Synthetic, Forward primer VMAT2
ggacaacatgctgctcactg

SEQ ID NO: 12 - DNA, 20 bp, Artificial
  Synthetic, Reverse primer VMAT2
attcccggtgaccatagtcg

SEQ ID NO: 13 - DNA, 20 bp, Artificial
  Synthetic, Forward primer TH
gaggggaaggccgtgctaaa

SEQ ID NO: 14 - DNA, 20 bp, Artificial
  Synthetic, Reverse primer TH
gaggcgcacgaagtactcca

SEQ ID NO: 15 - DNA, 20 bp, Artificial
  Synthetic, Forward primer NURR1
agaccttcgctgtgcccaac

SEQ ID NO: 16 - DNA, 20 bp, Artificial
  Synthetic, Reverse primer NURR1
tcccaacagccaggcacttc

SEQ ID NO: 17 - DNA, 20 bp, Artificial
  Synthetic, Forward primer FOXA1
gggctggatggttgtattgg

SEQ ID NO: 18 - DNA, 18 bp, Artificial
  Synthetic, Reverse primer FOXA1
atgttgccgctcgtagtc

SEQ ID NO: 19 - DNA, 20 bp, Artificial
  Synthetic, Forward primer FLJ21924
tggcggtaacagtccatcag

SEQ ID NO: 20 - DNA, 20 bp, Artificial
  Synthetic, Reverse primer FLJ21924
gcagttgggctagtagttgc

SEQ ID NO: 21 - DNA, 18 bp, Artificial
  Synthetic, Forward primer CBLN1
aatgagacggagcccatc

SEQ ID NO: 22 - DNA, 18 bp, Artificial
  Synthetic, Reverse primer CBLN1
tgaaagtgctgcgttctg

SEQ ID NO: 23 - DNA, 20 bp, Artificial
  Synthetic, Forward primer PITX2
accttacggaagcccgagtc

SEQ ID NO: 24 - DNA, 20 bp, Artificial
  Synthetic, Reverse primer PITX2
tggatagggaggcggatgta

SEQ ID NO: 25 - DNA, 20 bp, Artificial
  Synthetic, Forward primer IGF1
tggtggatgctcttcagttc

SEQ ID NO: 26 - DNA, 20 bp, Artificial
  Synthetic, Reverse primer IGF1
tccctctacttgcgttcttc

SEQ ID NO: 27 - DNA, 18 bp, Artificial
  Synthetic, Forward primer PITX3
gccgctactgggagtctg

SEQ ID NO: 28 - DNA, 18 bp, Artificial
  Synthetic, Reverse primer PITX3
ctgtgctcctggcccttg

SEQ ID NO: 29 - DNA, 21 bp, Artificial
  Synthetic, Forward primer DLK1
ttcacggactctgtggagaac

SEQ ID NO: 30 - DNA, 21 bp, Artificial
  Synthetic, Reverse primer DLK1
cgcagaaattgcctgagaagc

SEQ ID NO: 31 - DNA, 19 bp, Artificial
  Synthetic, Forward primer GFRa1
caagcacagctacggaatg

SEQ ID NO: 32 - DNA, 21 bp, Artificial
  Synthetic, Reverse primer GFRa1
ctgaggctactggagtctatg

SEQ ID NO: 33 - DNA, 20 bp, Artificial
  Synthetic, Forward primer Hs.130544
ctccaacatgtcgagtctac

SEQ ID NO: 34 - DNA, 18 bp, Artificial
  Synthetic, Reverse primer Hs.130544
ttgtgatggagccatacc

SEQ ID NO: 35 - DNA, 18 bp, Artificial
  Synthetic, Forward primer Hs.370410
gtgaggcttgttgcagaa

SEQ ID NO: 36 - DNA, 18 bp, Artificial
  Synthetic, Reverse primer Hs.370410
taatggcacctgctttgg

SEQ ID NO: 37 - DNA, 21 bp, Artificial
  Synthetic, Forward primer Hs.9887
tgtagatgggaacatggtttg

SEQ ID NO: 38 - DNA, 21 bp, Artificial
  Synthetic, Reverse primer Hs.9887
gcagtctaagtgaaacccata

SEQ ID NO: 39 - DNA, 22 bp, Artificial
  Synthetic, Forward primer Hs.27261
gtatgtctgtgcacggacaatg

SEQ ID NO: 40 - DNA, 22 bp, Artificial
  Synthetic, Reverse primer Hs.27261
gtttccctaacacagcgagttc

SEQ ID NO: 41 - DNA, 19 bp, Artificial
  Synthetic, Forward primer Hs.132591
ggtggctgactacattgtg

SEQ ID NO: 42 - DNA, 19 bp, Artificial
  Synthetic, Reverse primer Hs.132591
tgggtggaagatggaagag

SEQ ID NO: 43 - DNA, 19 bp, Artificial
  Synthetic, Forward primer Hs.30875
gtcaccagcctccaacttc

SEQ ID NO: 44 - DNA, 19 bp, Artificial
  Synthetic, Reverse primer Hs.30875
cccagattcggacaggatg

SEQ ID NO: 45 - DNA, 20 bp, Artificial
  Synthetic, Forward primer Hs.524341
ctacacgtccaaactgtctc

SEQ ID NO: 46 - DNA, 20 bp, Artificial
  Synthetic, Reverse primer Hs.524341
cagcataggctaggatgact

SEQ ID NO: 47 - DNA, 20 bp, Artificial
  Synthetic, Forward primer Hs.21374
cccacttggaataccaaacc

SEQ ID NO: 48 - DNA, 20 bp, Artificial
  Synthetic, Reverse primer Hs.21374
ttcactcccgagacattcac

SEQ ID NO: 49 - DNA, 20 bp, Artificial
  Synthetic, Forward primer B4GALT6
ccttttgccagccaagatac

SEQ ID NO: 50 - DNA, 20 bp, Artificial
  Synthetic, Reverse primer B4GALT6
agttctctcaaccggcatac

SEQ ID NO: 51 - DNA, 20 bp, Artificial
  Synthetic, Forward primer Hs.370410
accattcaccgccttcagcc

SEQ ID NO: 52 - DNA, 20 bp, Artificial
  Synthetic, Reverse primer Hs.370410
tgcctaagtcccgctcacac

SEQ ID NO: 53 - DNA, 20 bp, Artificial
  Synthetic, Forward primer Hs.517868
cgggcttcttcagaagtacc

SEQ ID NO: 54 - DNA, 20 bp, Artificial
  Synthetic, Reverse primer Hs.517868
aggaggagacacatggagag

SEQ ID NO: 55 - DNA, 21 bp, Artificial
  Synthetic, Forward primer Hs.302130
aaggaacgcaagggcttcaac

SEQ ID NO: 56 - DNA, 22 bp, Artificial
  Synthetic, Reverse primer Hs.302130
agacccaggaccagaatttgac

SEQ ID NO: 57 - DNA, 21 bp, Artificial
  Synthetic, Forward primer Hs.7413
gcccaacagattcctgtgatg

SEQ ID NO: 58 - DNA, 21 bp, Artificial
  Synthetic, Reverse primer Hs.7413
cgcactgtcttctcagtcaac

SEQ ID NO: 59 - DNA, 20 bp, Artificial
  Synthetic, Forward primer Hs.26608
tgccaaaccgtttcgtcatc

SEQ ID NO: 60 - DNA, 20 bp, Artificial
  Synthetic, Reverse primer Hs.26608
tgcaaacgacgtcaagaacc

(19) United States
(12) Patent Application Publication
(10) Pub. No.: US 2007/0128168 A1
(43) Pub. Date: Jun. 7, 2007

(54) Novel genes regulated in the developing human ventral mesencephalon

(75) Inventors: Jesper Roland Jorgensen, Frederiksberg C (DK); Lars U. Wahlberg, Asnaes (DK)

Correspondence Address: BROWDY AND NEIMARK, P.L.L.C.;624 NINTH STREET, NW, SUITE 300, WASHINGTON, DC, 20001-5303, US

(73) Assignee: NsGene A/S, Ballerup (DK)

(21) Appl. No.: 11/599,587

(22) Filed: Nov. 15, 2006

Related U.S. Application Data
(60) Provisional application No. 60/736,335, filed on Nov. 15, 2005.

(30) Foreign Application Priority Data
Nov. 15, 2005 (DK) 2005-01592

(51) Int. Cl. A61K 48/00; G01N 33/567; C12N 5/08; C12N 15/86

(52) U.S. Cl. 424/93.2; 435/368; 435/456; 435/7.2

(57) ABSTRACT

A human embryonal stem cell, neural stem cell, neural precursor cell, neural cell or dopaminergic neuron is genetically modified to overexpress at least one of certain genes identified as regulated in the developing human ventral mesencephalon, and more particularly, up-regulated in the ventral tegmentum. The genes are associated with dopaminergic differentiation.

The present invention relates to the field of generation of, manipulation of, and selection of dopaminergic neurons.

### BACKGROUND

In Parkinson's disease (PD), the degeneration of mesencephalic dopaminergic (mDA) neurons cause symptoms characterized by tremor, rigidity, and akinesia (Lang and Lozano, 1998b; Lang and Lozano, 1998a). Although symptomatic treatment is available and relatively effective during the early stages of the disease, the dopaminergic (DA) neuron degeneration continues and no disease modifying or long-term effective treatments are available. Transplantation of first trimester fetal mesencephalic tissue containing immature mDA neurons has demonstrated beneficial effects in Parkinson patients and is regarded as a proof-of-principle that neural replacement can work in the human brain (Winkler et al., 2005). However, both practically and ethically this approach is not a realistic large-scale treatment for the approximately 1% of the human population over the age of 50 affected by the disease (Polymeropoulos et al., 1996). In addition, neural transplantation for PD is not without problems, and dyskinetic side effects have been described that may be related to the heterogenous make-up of the transplanted tissues (Freed et al., 2001;Olanow et al., 2003). Therefore much research is currently geared towards finding alternative and more defined sources of DA neurons. In particular, stem/neural progenitor cells have received much attention as they have the potential to generate large numbers of DA neurons in a standardized and controlled fashion (Roybon et al., 2004). It has been shown in rodents (Kim et al., 2002;Studer et al., 1998; Bjorklund et al., 2002) and recently also in non-human primates (Takagi et al., 2005) that transplantation of DA neurons derived from stem cells can lead to symptomatic recovery in animal models of PD. However, the generation of similar cells from human sources has not been equally successful. Instead of replacing DA neurons through transplantation, an alternative therapeutic strategy for PD is to protect or regenerate the remaining endogenous DA neurons or their precursors that may exist in the adult brain. GDNF is one example of a potent factor important for the development of DA neurons in vivo (Lin et al., 1993) that have shown beneficial effects in PD models and patients (Kirik et al., 2004). Similarly, factors of importance for specifying the DA phenotype during development may have therapeutic potential if delivered to the relevant endogenous or transplantable precursor cells.

In higher vertebrates, the majority of DA neurons are located within the substantia nigra (SN) and the ventral tegmental area (VTA) in the mesencephalon. In the human embryo, immature DA neurons can be found in the ventral part of the tegmentum from approximately six weeks gestational age (GA) (Almqvist et al., 1996). They arise near the mid/hindbrain boundary (isthmus) and the floor plate by the combined actions of two secreted signaling proteins, fibroblast growth factor 8 and sonic hedgehog (Hynes and Rosenthal, 1999). Even though many factors involved in DA neuron specification and survival are known (Vitalis et al., 2005), the ontogeny of the system remains obscure. It is therefore necessary to identify factors and markers expressed during this developmental timeframe in order to learn to control the fate and survival of expandable DA neuron progenitors in cell replacement or regenerative treatment strategies for PD.

### SUMMARY OF THE INVENTION

In a first aspect, the invention relates to a human embryonal stem cell, a human neural stem cell, a human neural precursor cell, a human neural cell, or a human dopaminergic neuron being genetically modified to overexpress at least one gene selected from the group consisting of genes from Table 2, that are not marked with bold, and genes from Table 3, 4, and 6.

The genes identified in the present application are specifically overexpressed in human embryonal tissues associated with the differentiation of dopaminergic neuruons and their precursors. These cells, overexpressing a gene associated with dopaminergic differentiation may be used for therapeutic purposes or as an experimental tool in studying dopaminergic differentiation.

In one embodiment the cell is isolated from the human body.

Preferred genes from Tables 3 and 4 are those wherein the fold change in probe signal between VT and DT of the gene is above 1.5, preferably above 1.6, more preferably above 1.7, more preferably above 1.8, more preferably above 1.9, more preferably above 2.0.

In one embodiment the the gene has the GO annotation “signal transduction” or “binding” in Table 2.

One preferred group of genes are those selected from the group consisting of genes from Table 4 and 6.

In one embodiment, the gene is selected from the group consisting of transmembrane genes from Table 4. Preferably this gene is selected from the group consisting of KIAA1145, SLC10A4, SLC2A13, and LRRC3B.

In another embodiment the gene is selected from the group consisting of transcription factor genes from Table 4. Preferably this gene is selected from the group consisting of FU45455 and C20Orf100.

In another embodiment the gene is selected from the group consisting of genes from Table 6. Preferably this gene is selected from the group consisting of TNFRSF25, SLC25A29, MGC40499, NRN1, FU20519, FU20519, MGC61716, MGC61716, LOC387758, SPOCK3, SPOCK3, DKFZP564K1964, MGC21688, GRCA, and EGFL9. More preferably this gene is selected from the group consisting of TNFRSF25, SLC25A29, MGC40499, NRN1, FU20519, and FU20519. In another preferred embodiment of Table 6, the gene is is selected from the group consisting of OS-9, NRN1, C1QTNF4, C14orf112, SLC25A29, DKFZP564K1964, FAM19A2, and SPOCK3. More preferably this gene is selected from the group consisting of OS-9, NRN1, C1QTNF4, and C14orf112.

Preferably the gene encodes a mature part of said protein.

In another aspect the invention relates to a method for enhancing the generation of dopaminergic neurons, comprising administering to a human cell at least one protein encoded by a gene selected from the group consisting of genes from Table 2, that are not marked with bold, and genes from Table 3, 4, 5 and 6.

In a preferred embodiment of this aspect the human cell is selected from the group consisting of human embryonal stem cells, human neural stem cells, human neural precursor cells, human neurons, and human dopaminergic neurons.

In one embodiment the gene encodes a transcription factor.

In another embodiment the gene encodes a protein involved in signal transduction. In one embodiment said protein is administered as a protein formulation. Preferably, the formulation comprises the mature part of said protein

In one embodiment the the gene is selected from the group of genes from Table 6. Preferably the gene from Table 6 is selected from the group consisting of TNFRSF25, SLC25A29, MGC40499, NRN1, FU20519, FU20519, MGC61716, MGC61716, LOC387758, SPOCK3, SPOCK3, DKFZP564K1964, MGC21688, GRCA, and EGFL9. More preferably the gene from Table 6 is selected from the group consisting of TNFRSF25, SLC25A29, MGC40499, NRN1, FLJ20519, and FLJ20519. In another preferred embodiment the preferred gene from Table 6 is selected from the group consisting of OS-9, NRN1, C1QTNF4, C14orf112, SLC25A29, DKFZP564K1964, FAM19A2, and SPOCK3. More preferably gene is selected from the group consisting of OS-9, NRN1, C1QTNF4, and Cl4orf1 12.

In another embodiment the gene is selected from the group of genes from Table 5. Preferably the gene from Table 5 is selected from the group consisting of FGF13, CSPG5, HDGF, LASS1, IGF1, RABEP1, JAGI, FGF9, BMP2, BMP15, FGF6, GDF3, and PDGFB. More preferably the gene from Table 5 is selected from the group consisting of FGF13, CSPG5, HDGF, LASS1, and IGF1.

The protein may be administered by causing said gene to be overexpressed in said cell. Said overexpression may becaused by transducing or transfecting said cell with an expression vector coding for said gene. The transduction/transfection may be performed in vitro. The transduction/transfection may also be performed in vivo.

The vector may be a virus vector. Alternatively the cell may be transfected using lipofection, electroporation, or calcium phosphate transfection.

The differentiation method of the invention may further comprise the use of a standard dopaminergic differentiation protocol.

In another aspect the invention relates to a method for enriching a population of cells comprising dopaminergic neurons or dopaminergic precursor neurons, said method comprising labelling the cells with a label specific for the expression of at least one gene selected from the group consisting of genes from Table 2, that are not marked with bold, and genes from Table 3, 4 , 5 and 6 and sorting the cells.

The label may compsise an antibody. In another embodiment the label comprises a nucleotide probe.

In a preferred embodiment, the gene is selected from genes from Table 4. The gene may be annotated in Table 4 as a transmembrane gene, preferably selected from the group consisting of KIAA1145, SLC10A4, SLC2A13, and LRRC3B.

In another aspect the invention relates to a method of treatment of Parkinson's disease, said method comprising administering to a patient in need thereof a therapeutically effective amount of at least one protein encoded by a gene selected from the group consisting of genes from Table 2, that are not marked with bold, and genes from Table 3, 4, 5 and 6.

Preferably the gene is selected from the group consisting of genes from Table 3, 4 and 6.

In one embodiment, the gene is selected from genes from Table 4. The gene may be annotated in Table 4 as a transmembrane gene, preferably selected from the group consisting of KIAA1145, SLC10A4, SLC2A13, and LRRC3B. In another embodiment the gene is annotated in Table 4 as a transcription factor. Preferably said transcription factor is FLJ45455 and C20orf100.

In one embodiment the the gene is selected from the group of genes from Table 6. Preferably the gene from Table 6 is selected from the group consisting of TNFRSF25, SLC25A29, MGC40499, NRN1, FLJ20519, FLJ20519, MGC61716, MGC61716, LOC387758, SPOCK3, SPOCK3, DKFZP564K1964, MGC21688, GRCA, and EGFL9. More preferably the gene from Table 6 is selected from the group consisting of TNFRSF25, SLC25A29, MGC40499, NRN1, FLJ20519, and FLJ20519. In another preferred embodiment the preferred gene from Table 6 is selected from the group consisting of OS-9, NRN1, C1QTNF4, C14orf112, SLC25A29, DKFZP564K1964, FAM19A2, and SPOCK3. More preferably gene is selected from the group consisting of OS-9, NRN1, C1QTNF4, and C14orf112.

In another embodiment the gene is selected from the group of genes from Table 5. Preferably the gene from Table 5 is selected from the group consisting of FGF13, CSPG5, HDGF, LASS1, IGF1, RABEP1, JAG1, FGF9, BMP2, BMP15, FGF6, GDF3, and PDGFB. More preferably the gene from Table 5 is selected from the group consisting of FGF13, CSPG5, HDGF, LASS1, and IGF1.

The protein may be administered as a protein formulation.

In cases where the gene encodes a growth factor, and said growth factor may be administered by implanting a compositon of cells secreting said protein in the striatum or substantia nigra of a subject. Preferably, the cells are encapsulated behind a semipermeable immunoisolatory membrane.

In another embodiment the protein is administered by causing said gene to be overexpressed in said cell. Said overexpression may becaused by transducing or transfecting said cell with an expression vector coding for said gene. Said transduction/transfection may be performed in vivo, and preferably by using a virus vector.

### BRIEF DESCRIPTION OF THE DRAWINGS

FIG. 1. A) Illustration of midbrain dissections into dorsal and ventral tegmentum (DT and VT). Midbrain samples older than 7 weeks gestational age, were further sub dissected into VT and DT. We aimed at dissecting only part of the VT as close as possible to where dopaminergic neurons are expected. For comparison, the adjacent DT was used. In both cases the neighboring lateral tissue was discarded. B) RNA quality. The RNA isloated from sub-dissected tegmental samples for array analysis were analyzed using the the Agilent Bioanalyzer 2100. Note the clear 28S and 18S peaks ([28S/18S] ratio >2) and that there are no noise between the peaks or low molecular weight contaminations. This indicates overall high quality and non-degraded RNA samples.

FIG. 2. The relationship between P-values and log₂ fold change for all probe sets on HG-U133A and HGU133B shown as Volcano plots. High similarity between the expression profiles of VT and DT is evident from the very narrow plots. Only very few probes are changed more than two fold. All possible permutations were made, but not included in the plots. The arrow indicates ALDH1A1 with fold a change of 2.87 (log₂) and a P-value of 1.2×10⁷. The hatched areas indicate the low stringency filter used for identification of probes up regulated in VT.

FIG. 3. Verification of HG-U133A data by literature mining. Since the HG-U133A chip contain mostly known human genes, the existing literature can be used to evaluate the hit rate. The y-axis shows the percentage of probes verified by the existing literature as a function of the number of probes. As the top eight probes can be verified by the literature, the verification is 100% (8/8). Including the ninth probe, FLJ21924, which cannot be verified through literature mining, the verification drops to 88.9% (8/9). Including CBLN1 it drops even further to 80% (8/10) and so forth. See text for references. Vertical bars indicate >2 and >1.5 fold increased expression in VT respectively.

FIG. 4. Q-PCR expression profiles of known DA marker genes in first trimester human brain tissue. A panel of human fetal brain tissues 5 to 10 weeks GA was collected and sub dissected where possible. MES: mesencephalon, FBr: forebrain, DT: dorsal tegmentum and VT: ventral tegmentum. Lower case letters (a,b,c,d,e and f) indicate samples from the same biological cases respectively. Note the very similar expression profiles. FIG. 5. Clustering and fold change calculations. The Q-PCR expression profiles from FIG. 4 and FIG. 6 were clustered into two clusters (I and II) using ClustArray. Note that the known DA marker genes cluster together eventhough most samples demonstrate very similar expression profiles. For the 8w DT and VT samples parallel to the array analysis, a t-test was used to evaluate the statistical significance of differential expression. The average fold change (8w FC) and the P-value for each gene are shown in the table right to the diagram.

FIG. 6. Q-PCR expression profiles of novel genes in first trimester human brain tissue. The fifteen most regulated sequences from HG-U133B (except for EPHA5) and Cerebellin, PITX2, FU21924 and DLK1 selected from HG-U133A were analysed by Q-PCR. See legend to FIG. 4 for details.

FIG. 7. Q-PCR expression analysis of SLC1OA4, FLJ45455 and KIAA1145 on a large panel of adult human tissue.

FIG. 8. Expression analysis in developing mouse CNS. Tisue from different brain regions of E10.5, E11.5, E13.5, P1 and adult mice was dissected and RNA isolated. The expression panel consists of cDNA prepared from the following tissues; dorsal forebrain (DFB), ventral forebrain (VFB), ventral mesencephalon (VM), dorsal mesencephalon (DM) and spinal cord (SC) from 10.5 and 11.5 weeks old embryos. In addition, cortex (CTX), medial and lateral ganglionic eminences (MGE/LGE), DM, VM and SC from 13.5 weeks old embryos were included. Furthermore, from newborn mouse (P1), cerebellum (Cb), CTX, VM, DM, and Striatum (MGE/LGE) were used and finally Cb, CTX, VM, DM, and SC were used from adult mouse. Note the uniform GAPDH expression in contrast to the other differentially expressed genes.

### DETAILED DESCRIPTION

The present invention provides new uses of nucleotides and polypeptides encoded thereby. Included in the invention are uses of the nucleic acid sequences, their encoded polypeptides, antibodies, and other related compounds. The sequences are collectively referred to herein as “DA-gene nucleic acids” or “DA-gene polynucleotides” and the corresponding encoded polypeptides are referred to as “DA-polypeptides” or “DA-proteins.” Unless indicated otherwise, “DA-genes” is meant to refer to any of the genes disclosed herein as being associated with a Dopaminergic phenotype. Tables 2, 3, 4, 5, and 6 provides a list of the DA-genes. The invention also relates to splice variants, SNP variants, fragments, derivatives, analogs of the DA-proteins and DA-genes described herein.

“Fragments” provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, and are at most some portion less than a full length sequence. Fragments may be derived from any contiguous portion of a nucleic acid or amino acid sequence of choice.

“Derivatives” are nucleic acid sequences or amino acid sequences formed from the native compounds either directly, by modification, or by partial substitution. “Analogs” are nucleic acid sequences or amino acid sequences that have a structure similar to, but not identical to, the native compound, e.g. they differ from it in respect to certain components or side chains. Analogs may be synthetic or derived from a different evolutionary origin and may have a similar or opposite metabolic activity compared to wild type. Homologs are nucleic acid sequences or amino acid sequences of a particular gene that are derived from different species. Derivatives and analogs may be full length or other than full length. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the proteins of the invention under stringent, moderately stringent, or low stringent conditions.

“A polypeptide having a biologically-active portion of a DA-polypeptide” refers to polypeptides exhibiting activity similar, but not necessarily identical, an activity of a polypeptide of the invention, including mature forms, as measured in a particular biological assay, with or without dose dependency. A nucleic acid fragment encoding a “biologically-active portion of DA-protein” can be prepared by isolating a portion of the relevant ORF, that encodes a polypeptide having a DA-protein biological activity, expressing the encoded portion of the DA-protein (e.g., by recombinant expression in vitro) and assessing the activity of the encoded portion of the DA-protein.

A DA nucleic acid can encode a mature DA-polypeptide. As used herein, a “mature” form of a polypeptide or protein disclosed in the present invention is the product of a naturally occurring polypeptide, precursor form, or proprotein. The naturally occurring polypeptide, precursor or proprotein includes, by way of nonlimiting example, the full-length gene product encoded by the corresponding gene. Alternatively, it may be defined as the polypeptide, precursor or proprotein encoded by an ORF described herein. The product “mature” form arises, by way of nonlimiting example, as a result of one or more naturally occurring processing steps that may take place within the cell (host cell) in which the gene product arises. Examples of such processing steps leading to a “mature” form of a polypeptide or protein include the proteolytic cleavage of a signal peptide or leader sequence. Thus a mature form arising from a precursor polypeptide or protein that has residues 1 to N, in which an N-terminal signal sequence from residue 1 to residue M is cleaved, would have the residues from residue M+1 to residue N remaining. Further as used herein, a “mature” form of a polypeptide or protein may arise from a post-translational modification other than a proteolytic cleavage event. Such additional processes include, by way of non-limiting example, pro-peptide cleavage, glycosylation, myristoylation or phosphorylation. In general, a mature potypeptide or protein may result from the operation of only one of these processes, or a combination of any of them.

Identification of Novel DA-Genes and -Proteins

In this study, we have used genome-wide expression profiling of human developing ventral tegmentum (VT) and dorsal tegmentum (DT) in combination with Q-PCR to identify genes involved in the development of mDA neurons. The vast majority of the known DA marker genes were found, but more importantly, several known genes as well as uncharacterized and hypothetical transcripts could be added to the list of genes preferentially expressed in VT. Overall, including both HG-U133A and HG-U133B data, an exceptionally high hit rate was demonstrated, which is unusual for an array study. Furthermore, the tissue used in this study is unique not only because of its human origin and precise dissection, but also because of its clinical relevance. 6-8 weeks GA mesencephalic tissues from the ventral part of the tegmentum have demonstrated the best viability and function in transplantation studies (Winkler et al., 2005). Therefore the novel DA-genes presented in this study may play important roles in both the creation and evaluation of authentic DA cell lines as well as in regenerative treatment strategies for PD.

One of the primary challenges working with microarrays, and especially arrays covering the entire human genome, is to limit the number of false positives. For that, we used a low stringency data filter taking into account both expression level, fold change, and significance. For the filtered probes on HG-U133A with a fold change higher than 1.5, half of them could be verified by literature mining. However, it should be noted that verification through literature is often based on rodent data where an exact match to the human developmental stage used in the array analysis is not possible. Nevertheless, if a gene has been demonstrated to be specifically expressed by PA neurons, we took it as confirmation, regardless of the developmental stage and species. A thorough literature mining of each gene is a time-consuming process but, in our hands, was more informative than dividing all regulated genes into GO categories and look for regulated clusters. With the latter approach we observed an overrepresentation of transcription and signal transduction, as one might expect in a developmental setting. For HG-U133B data, literature mining is not an option as most probes represent ESTs, but the same data filter designed for HG-U133A can be used to identify differentially expressed transcripts. For all the filtered probes from HG-U133B from the top down to 1.56 fold regulation, we were able to verify differential expression by Q-PCR, confirming a very effective data filter. Hence, we may expect many of the genes identified on HG-U133A, with similar fold changes, also to be differentially expressed.

The concept of fold change is complicated when working with mixed cell populations. A gene could be regulated several fold in a sub-population like the DA neurons, but diluted out in the total RNA mix used for array or Q-PCR analysis. Therefore it may still be interesting to look at genes with very small changes.

We chose to verify the array data on a panel of subdissected brain tissue ranging from 5 to 10 weeks GA. The 8w samples used for Q-PCR verification are different biological samples from the ones analyzed by array. We did not observe a particular good correlation between the fold changes detected by the two techniques. However, this is not only because of biological variation between samples, but more due to the fact that two situations were compared where biologically relevant expression levels may be only expected in one of them. This is most distinct for the DA marker genes expected only to be expressed in VT. Since the dynamic range is much larger and in particular because the detection limit is much lower for Q-PCR compared to the array technique, a higher fold change with Q-PCR was often noted. However, by including samples for Q-PCR verification from different brain regions and of different ages, a spacial and temporal DA expression profile could be seen that was followed by all the known DA genes tested. Several sequences of unknown function also followed this DA signature. Of particular interest is SLC10A4 where virtually all ESTs are from neuroblastomas or glioblastomas. The vast majority of neuroblastomas produce catecholamines including dopamine and human glioblastomas contain stem cell like neural precursors (Galli et al., 2004). These data support a role of the uncharacterized membrane protein SLC10A4 in DA progenitors.

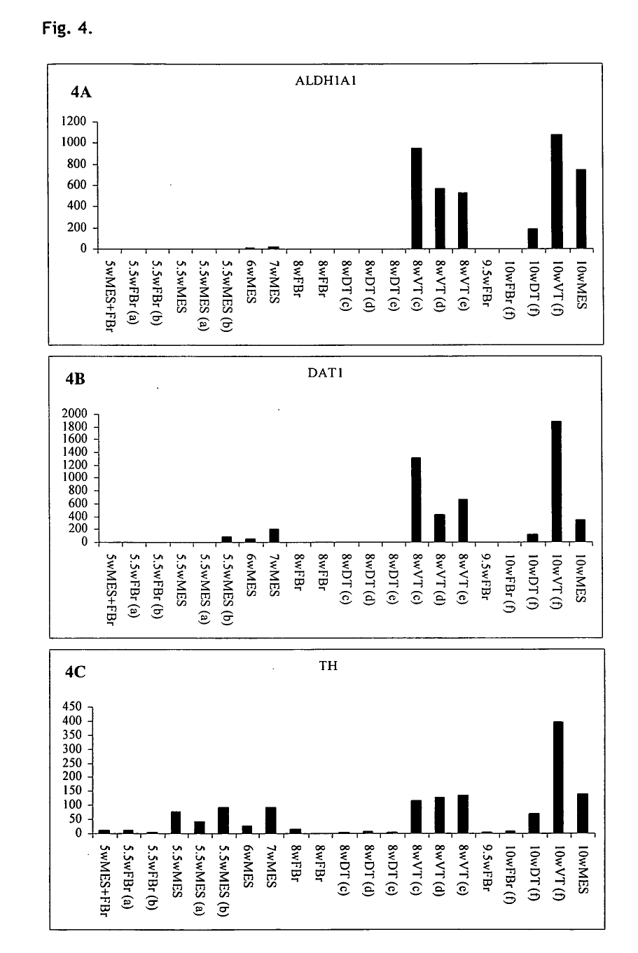

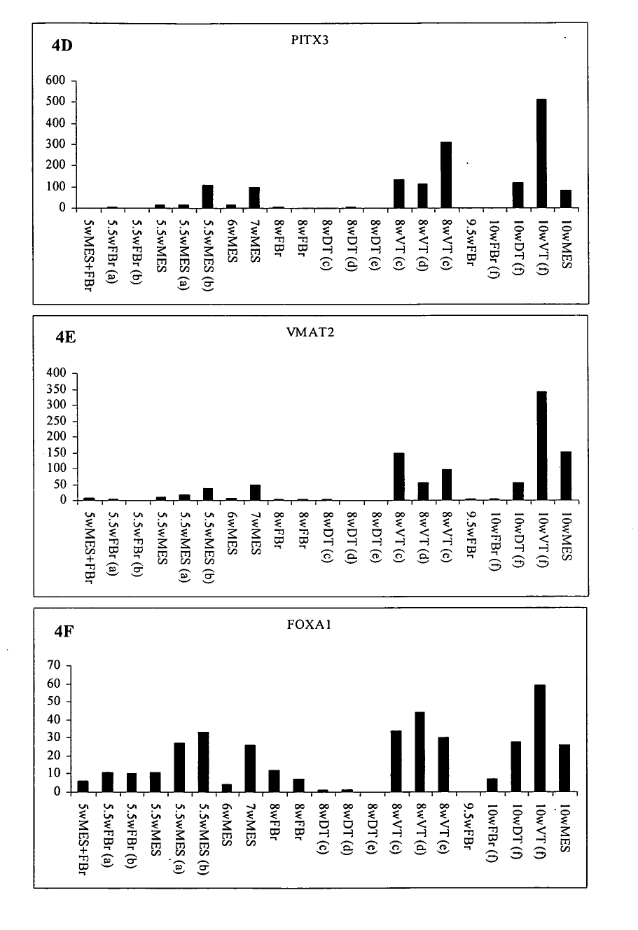

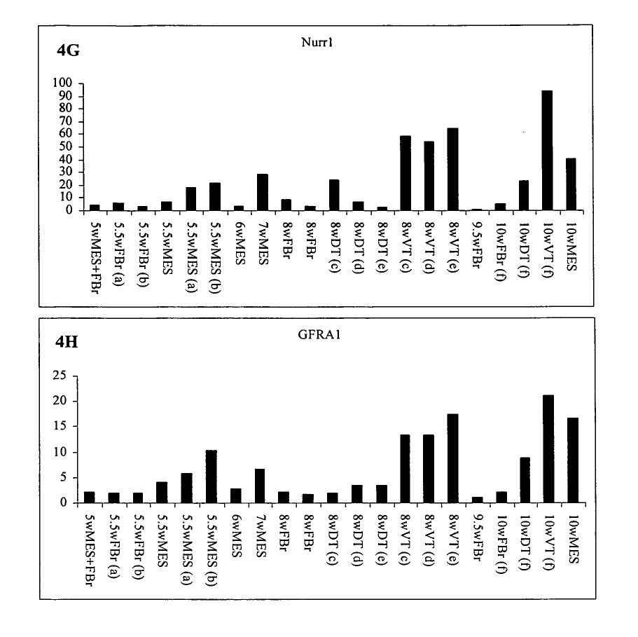

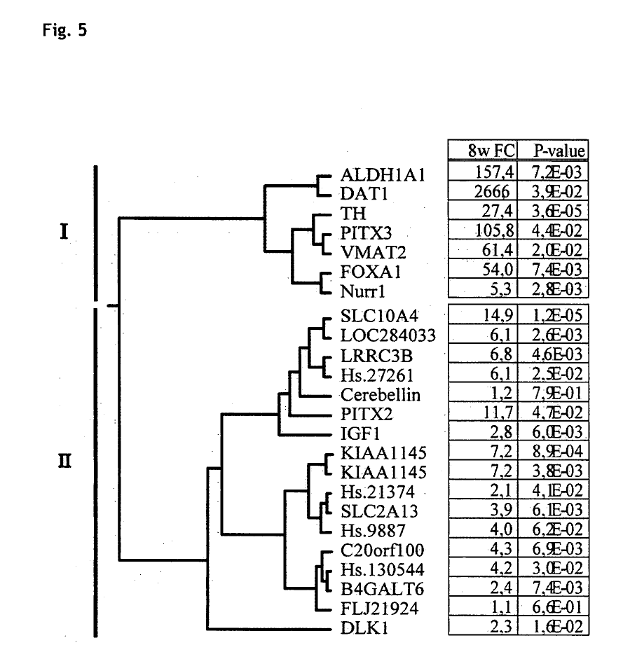

With a simple array setup, the vast majority of genes already known to be involved in DA development were identified. However, the GDNF family receptor alpha 1 (GFRA1) and the transcription factors LMXB1, EN1 and EN2 were not identified as differentially expressed between 8w human VT and DT. Both GFRA1 and LMXB1 were below the detection limit of the GeneChip, but when analyzed by Q-PCR, the expected expression profile of GFRA1 was similar to the other DA marker genes shown in FIG. 4 (data not shown). EN1 and EN2 appear in the neuroectoderm in mouse around E8 and will subsequently mark the midbrain/hindbrain border (Simon et al., 2004). Shortly after, mDA neurons gradually begin to express the two engrailed genes. In the adult, EN1 is highly expressed in all adult midbrain DA neurons whereas EN2 in only a subset (Simon et al., 2001). With this dynamic expression pattern, we did not know whether to expect the engrailed genes to be preferentially expressed in human 8w VT. However, from the chip, a very robust signal was observed for both engrailed genes. For EN1, there was no difference between VT and DT but EN2 showed 1.6 fold higher expression in DT.

To our knowledge, no genome-wide expression profiling of developing DA neurons using non-manipulated human material has been previously done. Expression profiles of laser microdissected adult rat DA neurons have recently been investigated by custom cDNA arrays to elucidate catecholaminergic diversity (Greene et al., 2005;Grimm et al., 2004). Although the purpose of theses studies were different from ours, data can be used to identify genes expressed by DA neurons in general and in particular by mDA neurons. However, the overlap with our data is limited to TH, VMAT, ALDH1A1, ZAKI-4, RAB3B and VGLUT2 for Grimm et al. (2004) and to Calbindin, DRD2, ADRAL B, BDNF and IGF1 for Greene et al. (2005). In another study also using adult rats, fluoro-gold labeling combined with laser capture microdissection was used to compare the expression profile of midbrain DA neurons to non-DA cortical neurons (Yao et al., 2005). Of the 18 genes identified as enriched in SN, the DA marker genes; TH, VMAT2 and ALDH1A1 overlapped with our study. Finally, in an attempt to find genes exclusive to the mDA cell population, PCR based differential display was used to compare mouse mDA neurons from embryonic wild type and EN1/2 double mutants to DA neurons from adult olfactory bulb (Thuret et al., 2004b). Of the five genes identified by Thuret et al. (2004) as restricted to mDA neurons, FOXA1 and ERBB4 were also identified in our study, whereas MAP1 B, EBF3 and SYT1 were all highly but not differentially expressed.

In conclusion, this study demonstrates the power of Affymetrix gene expression analyses in combination with Q-PCR on well-dissected human first trimester developing tissues to identify known and unknown genes with relevance for DA development. In addition, we have discovered known and unknown genes related to DA development that need to be studied further in hopes of discovering the ques necessary to push DA progenitors into functioning mDA neurons. These data could help to make cell replacement and regenerative therapies for PD a reality.

Categories of Genes

In the following there is provided a description of the different gene ontology classes used herein.

Secreted Growth Factors

Growth factor characteristics as used herein define sequence-related features similar to those of classical growth factors, which are secreted proteins acting on a target cell through a receptor to cause one or more of the following responses in the target cell: growth, proliferation, differentiation, survival, regeneration, migration, regain of function, improvement of function.

Secreted growth factors expressed at high levels in the ventral midbrain during embryo development at around the time of generation of dopaminergic precursor cells and/or dopaminergic neurons are likely to have an effect on the division, differentiation and survial of cells of the ventral midbrain. Therefore the mature part of the secreted growth factors may be used for expansion, survival and/or differentiation of neural cells in general, and cells from the ventral midbrain in particular. In particular, the secreted growth factors identified in the present invention in Tables 5 and 6 are likely to have an effect on the generation, migration, differentiation and/or survival of dopaminergic neurons and/or dopaminergic precrusor cells. Dopaminergic neurons of the human ventral midbrain are generated at around Gestational week 5-7 and migrate to their destination during the following weeks. This is exactly the period during which tissues for the expression analyses have been collected. Secreted growth factors with a high expression in the ventral midbrain during this period are likely to be involved in cell-cell signalling and are therefore likely to be involved in generation, migration, survival and/or differentiation of dopaminergic neurons.

As secreted growth factors exert their signalling effects on cells from the outside, a secreted growth factor can be formulated into a pharmaceutical composition or into a protein composition for in vitro use and be added to a composition of cells in vivo or in vitro and exert their effect on the cells.

Specifically the growth factors with high expression in the ventral midbrain at gestational age of 8 weeks include FGF13, CSPG5, HDGF, LASS1, IGF1, RABEP1, JAG1, FGF9, BMP2, BMP15, FGF6, GDF3, PDGFB, KITLG, TFF1, BMP1, INHBB, GDF5, HBEGF, TGFB1, FGF2, NODAL, and VWF.

Predicted growth factors with high expression in the ventral midbrain at gestational age of 8 weeks indluce TNFRSF25, SLC25A29, MGC40499, NRN1, FU20519, FU20519, MGC61716, MGC61716, LOC387758, SPOCK3, SPOCK3, DKFZP564K1964, MGC21688, GRCA, EGFL9, OS-9, FLJ10803, C14orf112, FAM19A2, MGC34647, C1QTNF4, WNT16, WNT16, CCNB1IP1, KITLG, and WNT7B.

Transmembrane Proteins

GO definition (Gene Ontology Consortium, Ashburner et al 2000): “Penetrating at least one phospholipid bilayer of a membrane. Also refers to the state of being buried in the bilayer with no exposure outside the bilayer. When used to describe a protein, indicates that all or part of the peptide sequence spans the membrane.”

Transmembrane proteins with a higher level of expression in the Ventral Tegmentum compared to the Dorsal Tegmentum have a number of possible utilities.

Proteins with a transmembrane domain are typically involved in signal transduction, e.g. as receptors, in cell-cell interaction by communicating with neighbouring cells, or as transporters of compounds across the cell membrane. Therefore, proteins with a transmembrane domain may be involved in regulating the generation, migration, survival and/or differentiation of dopaminergic neurons, which are generated in the Ventral Tegmentum at around the Gestational Age of the samples examined in the present application. Therefore such transmembrane proteins may be used in vitro and in vivo to enhance the generation and/or survival of dopaminergic neurons. The extracellular portion of such membrane proteins may be used as a protein therapeutic or may be added to an in vitro culture. The complete transmembrane proteins may be administered to a cell both in vitro and in vivo by transfection or transduction using expression vectors.

Transmembrane genes may be used as disease targets in one of the screening assays described herein to identity compounds, e.g. small molecules which may be used in the treatment of Parkinson's Disease.

Furthermore, as proteins with a transmembrane domain are bound to the cellular membrane, antibodies can be generated against the extraceltular part of the proteins. Such antibodies may be used for cell sorting. As the proteins with transmembrane domains of the present invention are believed to represent a population of cells that are either dopaminergic precursors or dopaminergic neurons, a population of cells enriched with respect to one or more of the transmembrane proteins described in Table 4, are likely to be enriched for dopaminergic precursor cells and/or dopaminergic neurons.

Such enriched cell populations may be used for experimental purposes or be used for replacement cell therapy wherein the enriched population is transplanted to the ventral midbrain of a subject diagnosed or predisposed with Parkinson's Disease.

Transcription

GO definition (Gene Ontology Consortium, Ashburner et al 2000): “The synthesis of either RNA on a template of DNA or DNA on a template of RNA”.

Transcription factors with a higher level of expression in the Ventral Tegmentum compared to the Dorsal Tegmentum have a number of possible utilities.

Transcription factors regulate gene transcription and may be potent regulators of differentiation. To some extent, transcription factors may also be involved in migration and survival. Therefore, transcription factors may be involved in regulating the generation, migration, survival and/or differentiation of dopaminergic neurons, which are generated in the Ventral Tegmentum at around the Gestational Age of the samples examined in the present application. Therefore such transcription factors may be used in vitro and in vivo to control the generation and of dopaminergic neurons. Transcription factors are most conveniently administered to a cell both in vitro and in vivo by transfection or transduction using expression vectors.

Signal Transduction

GO definition (Gene Ontology Consortium, Ashburner et al 2000): “The cascade of processes by which a signal interacts with a receptor, causing a change in the level or activity of a second messenger or other downstream target, and ultimately effecting a change in the functioning of the cell.”

Genes with a “Signal transduction” annotation may have utilities similar to those for secreted growth factors and/or proteins with transmembrane domains. Signal transduction genes may be used as disease targets in one of the screening assays described herein to identity compounds, e.g. small molecules which may be used in the treatment of Parkinson's Disease. Signal transduction genes may also be used for diagnosis of Parkinson's Disease.

Binding

GO definition (Gene Ontology Consortium, Ashburner et al 2000): “The selective, often stoichiometric interaction of a molecule with one or more specific sites on another molecule.”

Genes with a “binding” annotation may have utilities similar to those for secreted growth factors and/or proteins with transmembrane domains. Signal transduction genes may be used as disease targets in one of the screening assays described herein to identity compounds, e.g. small molecules which may be used in the treatment of Parkinson's Disease.

Transporter Activity

GO definition (Gene Ontology Consortium, Ashburner et al 2000): “Enables the directed movement of substances (such as macromolecules, small molecules, ions) into, out of, within or between cells.”

Genes with the GO annotation “transporter” may be involved in the generation and/or differentiation of dopaminergic precursor cells or dopaminergic neurons and may have a direct role in this process. Therefore overexpression of the relevant transporter gene in a stem cell, such as an embryonal stem cell or a neural precursor or stem cell may enhance the generation of dopaminergic precursors and/or dopaminergic neurons.

Transporter genes involved in the dopaminergic pathway, may also be used as disease targets in the development of treatments of Parkinson's Disease. A further utility is a diagnostic use in connection with diagnosis of Parkinson's Disease.

Catalytic ctivity

GO definition (Gene Ontology Consortium, Ashburner et al 2000): “Catalysis of a biochemical reaction at physiological temperatures. In biologically catalyzed reactions, the reactants are known as substrates, and the catalysts are naturally occurring macromolecular substances known as enzymes. Enzymes possess specific binding sites for substrates, and are usually composed wholly or largely of protein, but RNA that has catalytic activity (ribozyme) is often also regarded as enzymatic.”Genes with the GO annotation “Catalytic” may include enzymes. Enzymes that are involved in the generation and/or differentiation of dopaminergic precursor cells or dopaminergic neurons may have a direct role in this process. Therefore overexpression of the relevant catalytic gene in a stem cell, such as an embryonal stem cell or a neural precursor or stem cell may enhance the generation of dopaminergic precursors and/or dopaminergic neurons.

Catalytic genes involved in the dopaminergic pathway, may also be used as disease targets in the development of treatments of Parkinson's Disease. Catalytic DA-genes may also be used for diagnosis of Parkinson's Disease.

Structural Molecule Activity

GO definition (Gene Ontology Consortium, Ashburner et al 2000): “The action of a molecule that contributes to the structural integrity of a complex or assembly within or outside a cell.”

Genes with the GO annotation “structural” may be involved in the generation and/or differentiation of dopaminergic precursor cells or dopaminergic neurons and may have a direct role in this process. Therefore overexpression of the relevant structural gene in a stem cell, such as an embryonal stem cell or a neural precursor or stem cell may enhance the generation of dopaminergic precursors and/or dopaminergic neurons.

Structural genes involved in the dopaminergic pathway, may also be used as disease targets in the development of treatments of Parkinson's Disease.

Enzyme Regulator Activity

GO definition (Gene Ontology Consortium, Ashburner et al 2000): ∂Modulates the activity of an enzyme.”

Genes with an “enzyme regulator” annotation may have utilities similar to those for transcription factors. Enzyme regulator genes may be used as disease targets in one of the screening assays described herein to identity compounds, e.g. small molecules which may be used in the treatment of Parkinson's Disease. Enzyme regulator DA-genes may also be used as markers, e.g. for the diagnosis of Parkinson's Disease.

Preferred DA-Genes of the Invention

Looking at the expression of individual transcripts from 5w to low in FIG. 4, it is evident than ALDH1A1, DAT1, TH, PITX3, VMAT2, FOXAL and NURR1 representing known DA marker genes have very similar expression profiles. This group of genes cluster together in the top of the diagram in FIG. 5. For this group of genes the differential expression between VT and DT is very distinct, and a robust signal is observed from the younger midbrain samples coinciding with the time of DA induction in the human fetus (Almqvist et al., 1996;Verney, 1999).

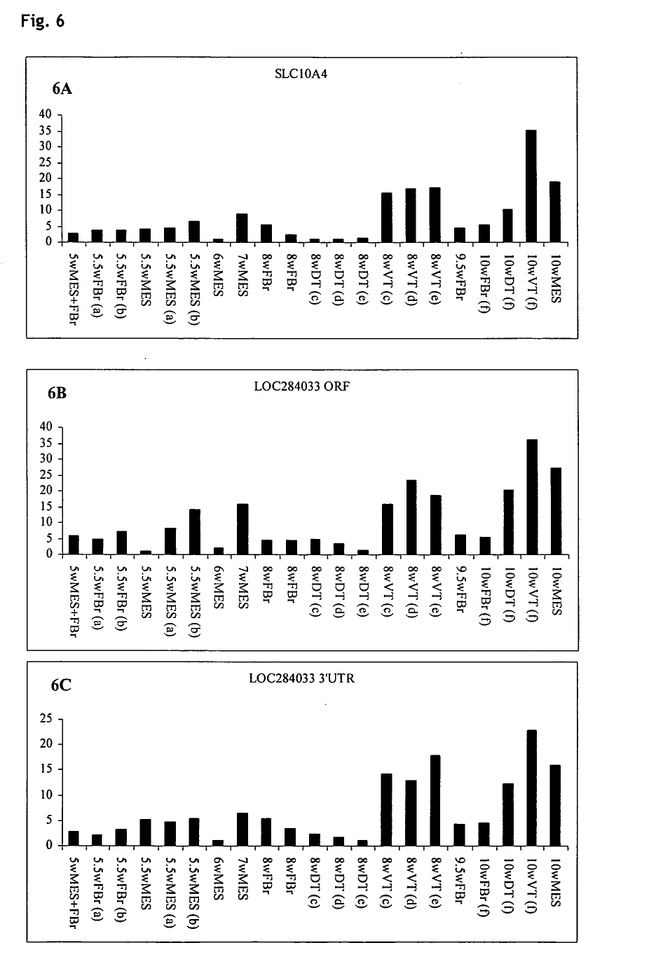

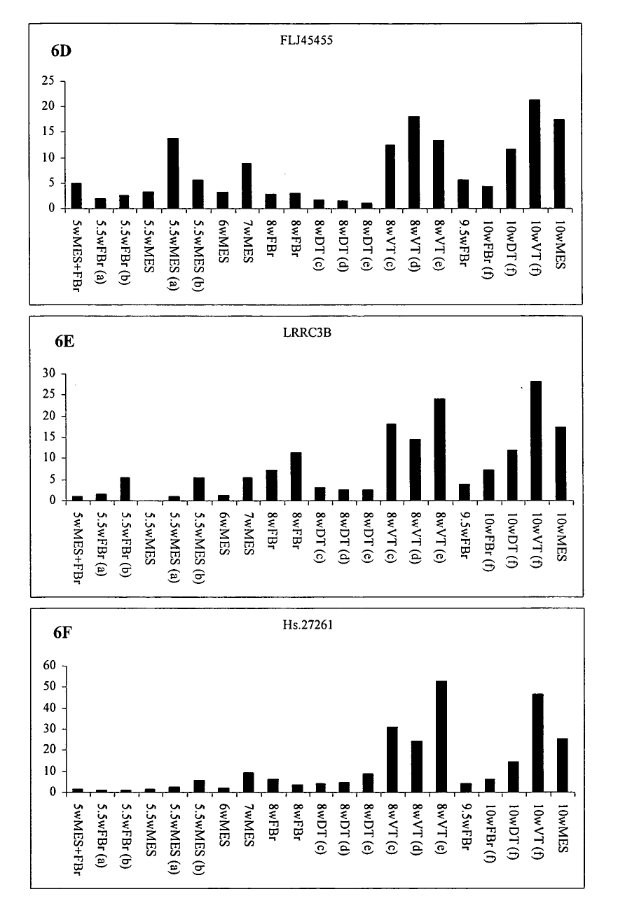

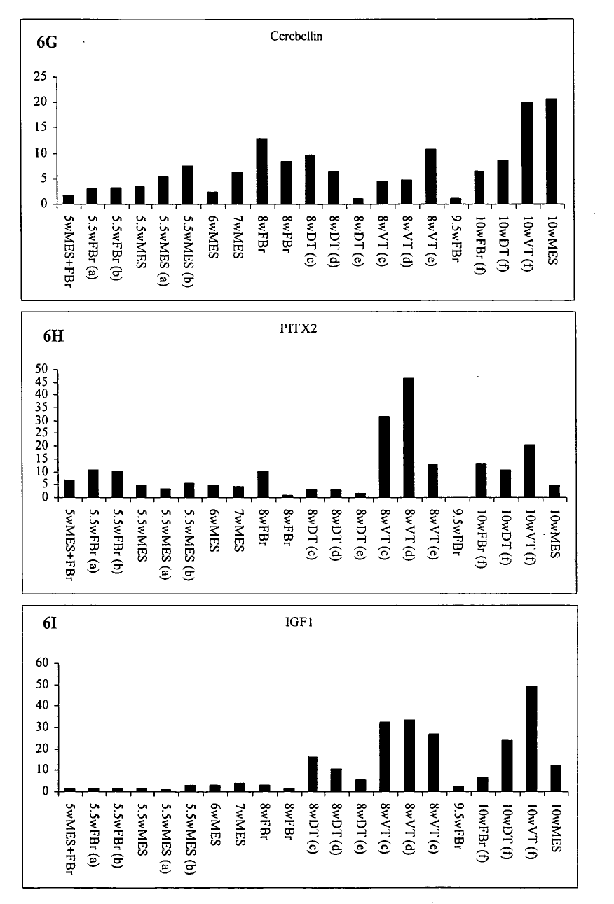

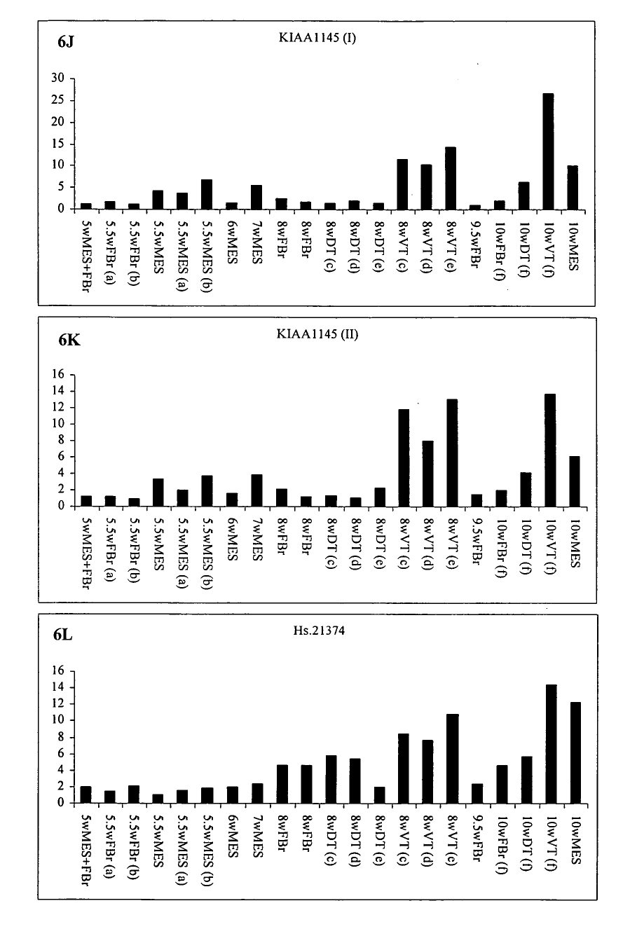

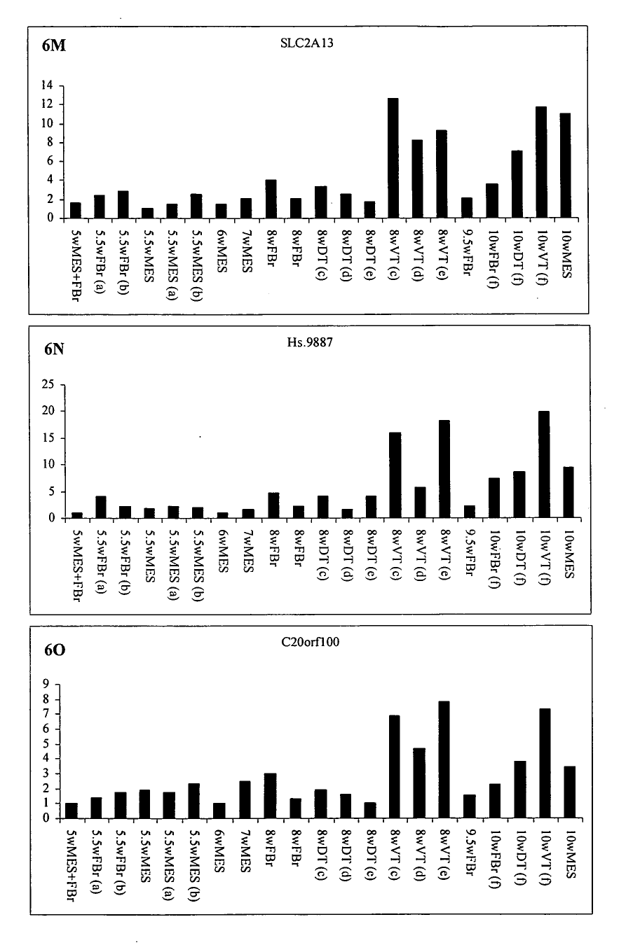

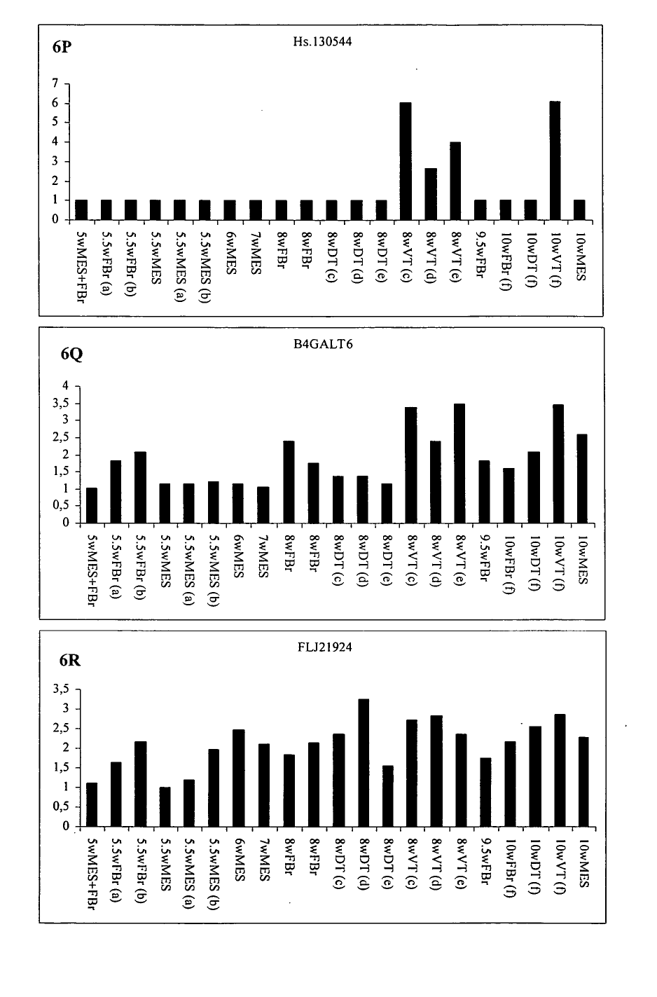

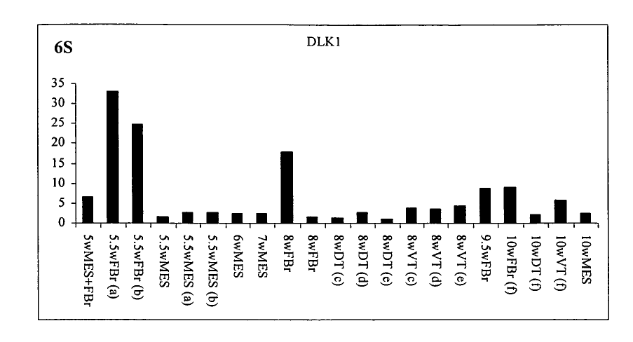

All the genes not previously associated with the DA phenotype are found in the second cluster (II) (FIG. 5). Here the DA signature is still strong for several genes, but in general the overall differential signal is weaker compared to group I (compare FIG. 4 and 6). These genes represent one group of preferrerd DA-genes encoding preferred DA-proteins of the invention.

SLC10A4

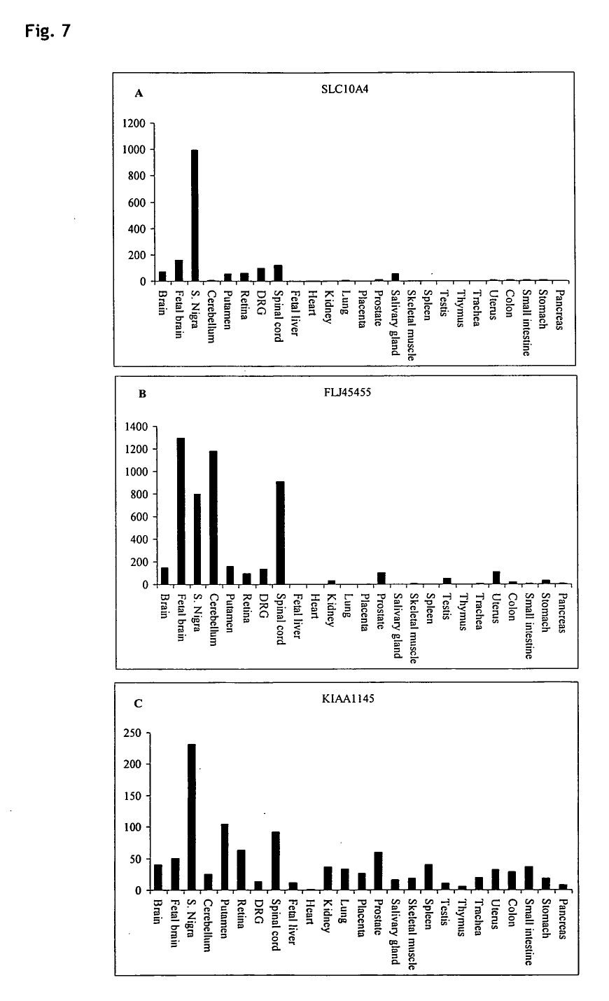

With a large and very significant up regulation in VT at 8w and increased expression in the young midbrain samples, the DA signature is most evident for SLC10A4 (FIG. 6A). This is a type 2 membrane protein belonging to the sodium/bile acid cotransporter family. Q-PCR expression analysis on several adult and a few fetal tissues revealed expression only in the CNS with particular high expression exclusively in substantia nigra (FIG. 7A).

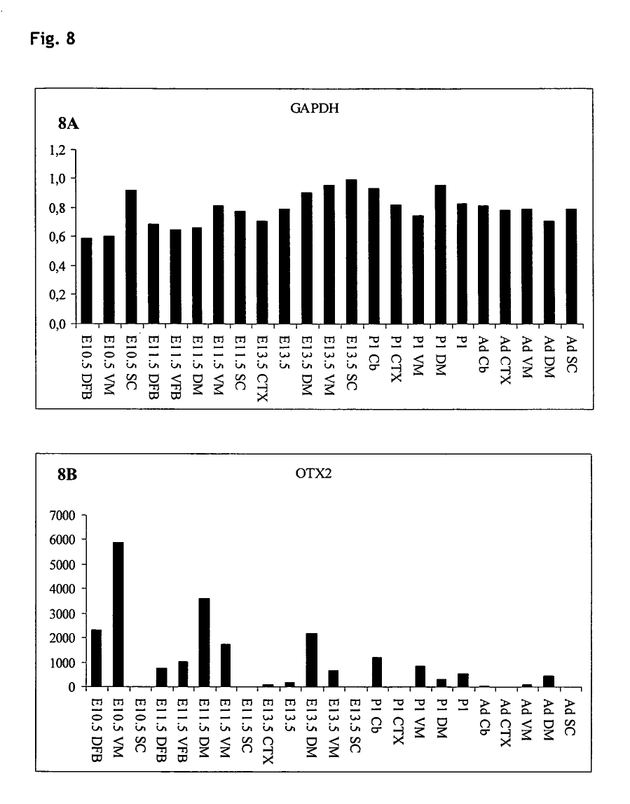

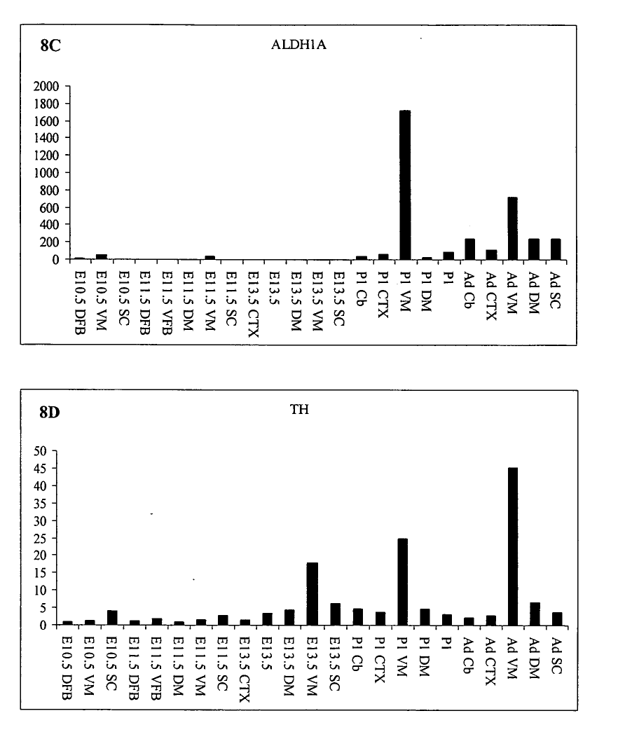

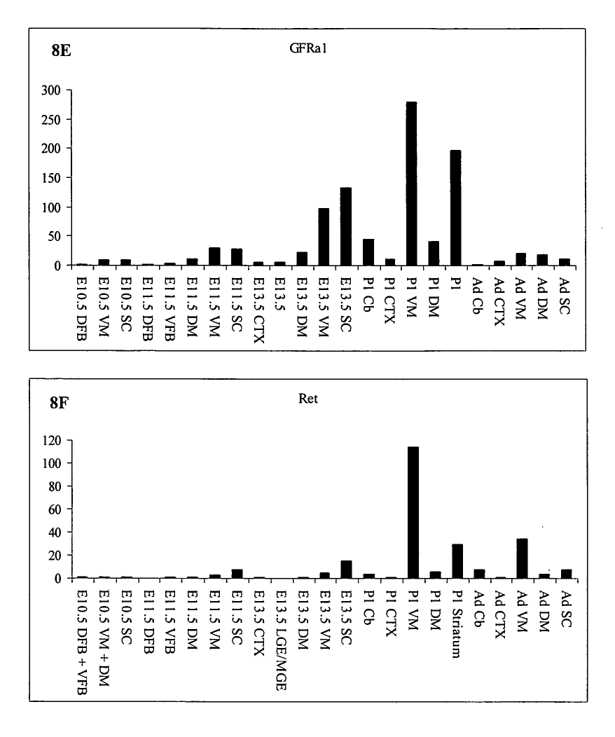

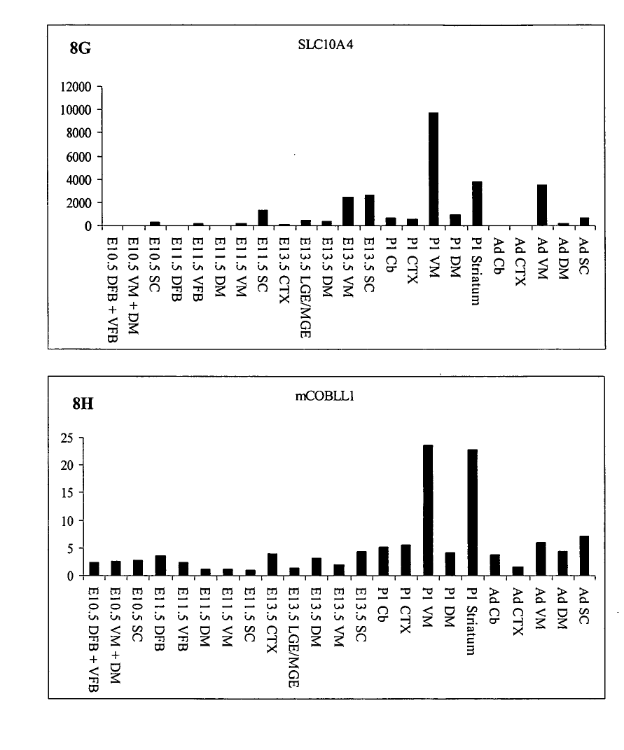

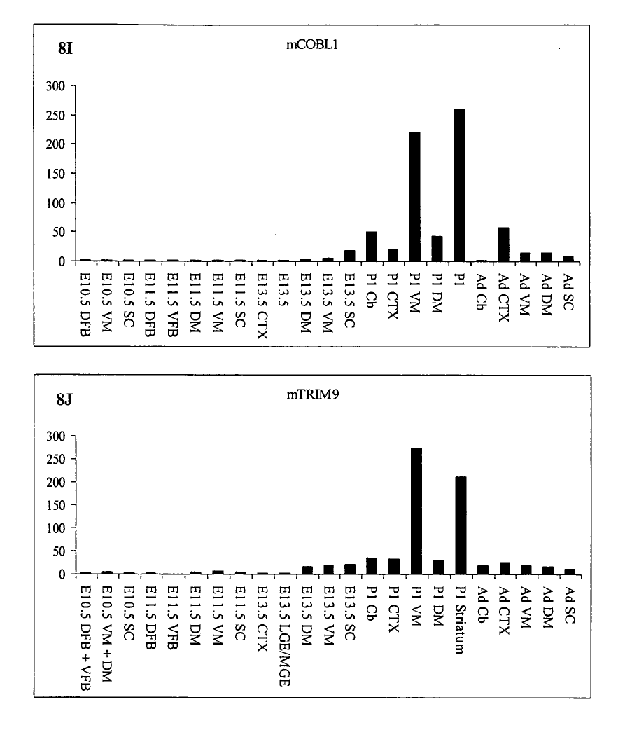

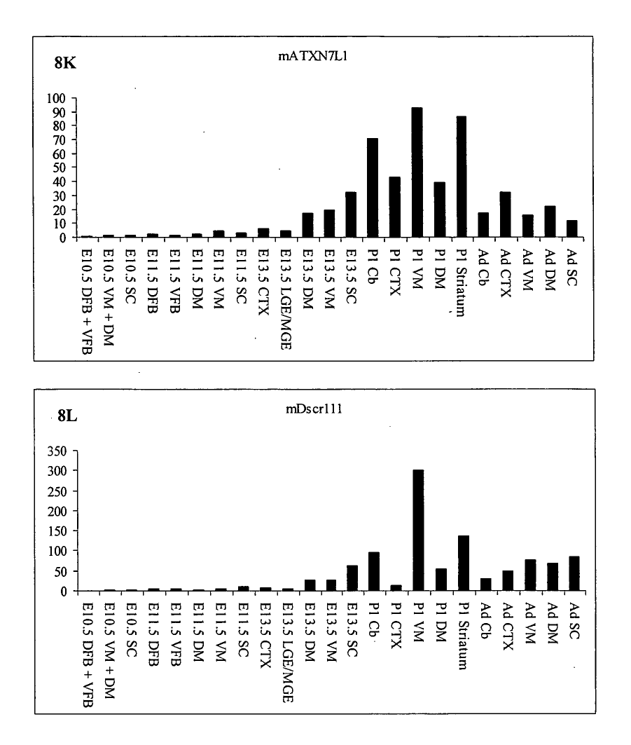

The GDNF receptors, GFRA1 and RET are primarily expressed in the ventral midbrain and spinal cord during development (FIG. 8E-F), in agreement with previous studies (Golden et al., 1999). Further, at P1, increased expression of GFRA1 and RET is observed in the striatum. SLC10A4 is very similar to the expression profiles of GFRA1 and RET with expression in spinal cord and ventral midbrain during development and later, at P1, increased expression in striatum (FIG. 8G). Interestingly, these areas of the brain constitute the nigostriatal pathway, which is a neural pathway connecting the substantia nigra with the striatum. It is one of the major dopamine pathways in the brain, and is particularly involved in the control of movement.

SLC10A4 is a cell surface gene. Therefore, antibodies can be generated against an extracellular epitope of SLC10A4 and a composition of cells comprising SLC10A4 expressing cells can be subjected to cell sorting resulting in a composition of cells enriched in SLC10A4 expressing cells and a composition of cells diminished in cells expressing SLC10A4. As SLC10A4 is a transporter protein, an fluorgenic substrate capable of being transported by SLC10A4 across the cell membrane can be used for in vivo monitoring of the level of SLC10A4 expression, similarly to the flurodopa assay known in the art. As SLC1OA4 is associated with the dopaminergic phenotype such an assay can be used for diagnosis of Parkinson's Disease, which involves loss of dopaminergic neurons, and for monitoring the effect of a clinical trial. Furthermore, a screening assay based on SLC1OA4 can be used to identify small molecules agonists and antagonists of SLC10A4, which may be used in developing therapies for Parkinson's Disease.

LRRC3B

LRRC3B, encoding an extremely well conserved leucine-rich repeat-containing protein with a predicted C-terminal transmembrane domain, is differentially expressed between VT and DT in samples older that 8w (FIG. 6E). However, it also seems to be expressed in forebrain and in samples younger than 8w only very limited differential expression is seen.

LRRC3B is a cell surface gene. Therefore, antibodies can be generated against an extracellular epitope of LRRC3B and a composition of cells comprising LRRC3B expressing cells can be subjected to cell sorting resulting in a composition of cells enriched in LRRC3B expressing cells and a composition of cells diminished in cells expressing LRRC3B. Furthermore, a screening assay based on LRRC3B can be used to identify small molecules agonists and antagonists of LRRC3B, which may be used in developing therapies for Parkinson's Disease.

KIAA1145

KIAA1145 identified by two different probes as higher expressed in VT, follow the DA expression profile in great detail when verified with Q-PCR (FIG. 6J-K). As the two different primersets used are detecting the same transcript, it is noteworthy that they also cluster together in FIG. 5. Q-PCR analysis on a panel of adult tissues demonstrated ubiquitous expression but noticeably the highest expression was seen in SN and putamen (FIG. 7C). KIAA1145 encodes a highly conserved transmembrane protein with coiled-coil domains.

KIAA1145 is a cell surface gene. Therefore, antibodies can be generated against an extracellular epitope of LRRC3B and a composition of cells comprising KIAA1145 expressing cells can be subjected to cell sorting resulting in a composition of cells enriched in KIAA1145 expressing cells and a composition of cells diminished in cells expressing KIAA1145. Furthermore, a screening assay based on KIAA1145 can be used to identify small molecules agonists and antagonists of KIAA1145, which may be used in developing therapies for Parkinson's Disease. SLC2A13

For the glucose transporter SLC2A1 3, differential expression was seen between VT and DT in samples from 8w, but no differential expression was observed between the younger samples (FIG. 6M).

SLC2A13 is a cell surface gene. Therefore, antibodies can be generated against an extracellular epitope of SLC2A13 and a composition of cells comprising SLC2A13 expressing cells can be subjected to cell sorting resulting in a composition of cells enriched in SLC2A13 expressing cells and a composition of cells diminished in cells expressing SLC2A13. As SLC2A13 is a transporter protein, an fluorgenic substrate capable of being transported by SLC2A13 across the cell membrane can be used for in vivo monitoring of the Level of SLC2A13 expression. As SLC2A13 is associated with the dopaminergic phenotype such an assay can be used for diagnosis of Parkinson's Disease, which involves loss of dopaminergic neurons, and for monitoring the effect of a clinical trial. Furthermore, a screening assay based on SLC2A13 can be used to identify small molecules agonists and antagonists of SLC2A13, which may be used in developing therapies for Parkinson's Disease.

FU45455

The Q-PCR analysis of LOC284033 expression also revealed a DA-like profile in the developing human brain (FIG. 6B). Using ProtFun (Jensen et al 2003) and SignalP (Nielsen et al, 1997; Bendtsen et al, 2004) (www.cbs.dtu.dk), the 123 amino acid large hypothetical protein LOC284033, is predicted as a growth factor with a probable signal peptide cleavage site. Q-PCR on several adult and few fetal human tissues revealed expression only in the CNS with particular high expression in adult SN, cerebellum and spinal cord ion addition to the fetal brain (FIG. 7B). However, mouse and rat homologues could not be found and neither did we succeed in heterologous expression of histidine tagged LOC284033.

Therefore it was speculated that LOC284033 might represent the UTR region of the neighboring well-conserved FU45455 less than 3 kb away. This was supported by Q-PCR analysis with primers in separate exons of FU45455 revealing a DA-like expression profile 35 virtually identical to that of LOC284033 (FIG. 6B-D).

FFU45455 encodes a transcription factor. The data presentere herein indicate that FU45455 is associated with a Dopaminergic phenotype. Therefore, FU45455 may be directly involved in differentiation of precursor cells into dopaminergic neurons. Therefore one utility of the FU45455 gene includes a method for differentiation of precursor cells into dopaminergic neurons by transfecting or transdudng a human cell with a FU45455 encoding construct. This method in one aspect may include in vivo gene therapy directed at treating, preventing or ameliorating Parkinson's Disease. C20orf100

For C20orf100, differential expression was seen between VT and DT in samples from 8w, but no differential expression was observed between the younger samples (FIG. 60). C20orf100 is only found in a single copy on chromosome 20, has both mouse and rat homologues and an ORF with a high mobility group box as found in many transcription factors.

C20Orf100 encodes a transcription factor. The data presented herein indicate that C20orf100 is associated with a Dopaminergic phenotype. Therefore, C20orf100 may be directly involved in differentiation of precursor cells into dopaminergic neurons. Therefore one utility of the C20orf100 gene includes a method for differentiation of precursor cells into dopaminergic neurons by transfecting or transducing a human cell with a C20orf100 encoding construct. This method in one aspect may include in vivo gene therapy directed at treating, preventing or ameliorating Parkinson's Disease.

Preferred growth factors with high expression in the ventral midbrain Preferred among the growth factors in Table 5 are those with a high probe signal, which is indicative of a high level of expression in the ventral midbrain. These preferred growth factors include: FGF13, CSPG5, HDGF, LASS1, IGF1, RABEP1, JAG1, FGF9, BMP2, BMP15, FGF6, GDF3, and PDGFB. More preferably the growth factors include: FGF13, CSPG5, HDGF, LASS1, and IGF1. These growth factors are well characterised growth factors.

The ProtFun score given in Table 6 indicates the likelihood that the encoded polypeptide is a growth factor. The higher the score the more certain the prediction is. Preferred factors include those with a ProFun odds score above 5 (TNFRSF25, SLC25A29, MGC40499, NRN1, FLJ20519, FU20519, MGC61716, MGC61716, LOC387758, SPOCK3, SPOCK3, DKFZP564K1964, MGC21688, GRCA, and EGFL9). More perferred factors include those with a ProtFun score above 7 (TNFRSF25, SLC25A29, MGC40499, NRN1, FU20519, and FLJ20519). In another embodiment, the preferred factors include those with the highest probe signal, which is indicative of a high expression level in the ventral midbrain. According to this embodiment, preferred growth factors include OS-9, NRN1, C1QTNF4, C14orf112, SLC25A29, DKFZP564K1964, FAM19A2, and SPOCK3. More preferably the growth factors include OS-9, NRN1, C1QTNF4, and C14orf112.

Verification of Activity and Association with the DA Phenotype

The association of the DA-genes of the present invention with the Dopaminergic phenotype as well as the involvement of the DA-genes in the development of the DA-phenotype can be verified using techniques known to persons skilled in the art.

In situ hybridization with TH co-stain would be the method to unquestionably reveal the DA nature of the genes identified in this study. Furthermore, it would be interesting to study the expression of these sequences in animal models with loss of DA neurons like the weaver mouse (Schmidt et al., 1982), the PITX3 deficient aphakia mouse (Nunes et al., 2003) or the knock-out animals of NURR1 (Zetterstrom et al., 1997), EN1/EN2 (Simon et al., 2001) or LMX1B (Smidt et al., 2000).

Knock-Out Mice

Gene inactivation studies for some of the DA-genes could shed more light on the at [east three separate pathways involved in DA development (Burbach et al., 2003;Vitatis et al., 2005). Methods for generating knock-out mice are well described in literature. Once knock-out mice have been generated, homozygous and heterozygous offspring can be bred and the phenotype of the brains can be studied. A defective development of the midbrain and/or the dopaminergic neurons indicated that the knocked-out gene plays a central role in the development of the midbrain and/or the dopaminergic neurons and that the gene and encoded protein may have pharmaceutical potential.

In Vitro Assays

An effect on the generation of dopaminergic neurons can be assayed in one of several well-described in vitro assays. These include e.g. the bioassay for survival of rat embryonic dopaminergic neurons described in Example 6 of U.S. Pat. No.6,734,284 or the assay testing the effect on survival of slice cultures of pig embryonic dopaminergic ventral mesencephatic neurons described in Example 7 of U.S. Pat. No. 6,734,284.

In Vivo Assays

There are a number of well-described animat models of Parkinson's Disease, which can be used to verify the effect of genes of the present invention on Parkinson's Disease. One particularly preferred assay is the 6′OHDA rat model as described in Rosenblad et al 2000, Mol Cell Neurosci, 15:199-214. Another animal model of Parkinson's Disease is the α-synoctein model, which is available both in rats and primates (Kirik et al, 2002, J Neurosci, 22:2780-91; Kirik et al, 2003, PNAS, 100:2884-2889).

Compositions of Cells

In connection with the present invention the expression “neural cells” means cells of any neural tissue in a mammal, including cells from the Central Nervous System (CNS) and the peripheral and autonomic nervous system, including cells of the adrenal medulla and ganglion cells of the gut.

The expression “primary neural cells” means cells as cottected from the mammal without any in vitro cultivation thereof. The population of primary neural cells may be any mixture of cells. Preferably, the population of neural cells collected from the mammals in the form of a cett suspension, wherein the cells have been dissociated so as to be present as single cells. The dissociation may be effected by any conventional method and equipment suitable for dissociation, such as mechanical dissociation.

The present invention relates to a method of obtaining a cell population enriched or diminished in DA-gene expressing cells comprising the steps of:

a) combining a starting population containing cells originating from mammalian neural cells with antibodies which bind specifically to an extracellular epitope of a DA-polypeptide to produce a first cell mixture,

b) removing unbound antibodies from the first cell mixture to produce a second cell mixture, and

c) separating cells comprising antibodies against an extracellular epitope of a DA-polypeptide from the second cell mixture to produce a cell population enriched in DA-gene expressing cells and a cell population diminished in DA-gene expressing cells.

In a preferred embodiment of the method of the invention, the cells of the starting population originate from human tissue. The cells of the starting population may originate from both fetal and adult tissue, preferably fetal tissue. Preferably, the cells of the starting population originate from the CNS and from subdissected fragments thereof.

The antibodies specific for a protein of the invention may be polyclonal or monoclonal antibodies, preferably the specific antibodies are monoclonal.

Preferably, the antibodies are labelled. In one embodiment of the invention the separation of cells comprising antibodies is carried out by a mechanical cell sorter.

In a preferred embodiment of the invention the specific antibodies are coupled to a fluorescent labelling compound. In this case the separation of cells comprising antibodies is preferably carried out using a fluorescense-activated cell sorter (FACS).

In a further preferred embodiment of the invention the antibodies are biotinylated. A particularly preferred variant of this embodiment is a method, wherein prior to step c) the second cell mixture is contacted with a streptavidin-fluorochrome or a avidin-flurochrome, and wherein the separation of cells comprising antibodies is carried out using a fluorescense-activated cell sorter (FACS). An alternative variant of the said embodiment is a method, wherein prior to step c) the second cell mixture is contacted with streptavidin or avidin linked to a particle, and wherein the separation of cells comprising antibodies is carried out by separating the particulate phase from the liquid phase.

In a further preferred embodiment of the invention, the antibodies are linked to a solid particle. Preferably, the solid particle is a magnetic particle. In this embodiment of the invention, the separation of cells comprising antibodies is preferably carried out by separating the particulate phase from the liquid phase.

A further preferred embodiment of the invention is a method, wherein prior to step c) the second cell mixture is contacted with an antibody to the specific antibody linked to a particle, and wherein the separation of cells comprising antibodies is carried out by separating the particulate phase from the liquid phase. Preferably, the particle is a magnetic particle.

A further preferred embodiment of the invention is a method, wherein the cells of the starting population are adherent cells cultivated on a solid support, and wherein the removal of unbound antibodies is carried out by rinsing.

A further preferred embodiment of the invention is a method, wherein the cells of the starting population are cultivated in suspension, and wherein the removal of unbound antibodies is carried out by centrifugating the first cell mixture and separating off the resulting supernatant.

A further preferred embodiment of the invention is a method, wherein the starting cell population is subjected to a further cell sorting procedure to enrich or diminish the cell population in cells expressing at least one further lineage specific marker. The further lineage specific marker may i.a. be nestin, glial fibrillary acidic protein (GFAP), vimentin, CD133, β3-tubulin and tyrosine hydroxylase (TH), 5E12′, CD24′, CD34-, CD45′. Preferably, the further lineage specific marker is CD133.

The antibodies of the subject invention can be labelled according to standard methods known in the art. For example, antibodies can be labelled with detectable labels such as fluorescein, rhodamine or with radioactive isotopes, or with biotin. Biotin binds strongly and irreversible to avidin. Biotinylated antibodies may be visualized by incubation with conjugates consisting of horseradish perioxidase and biotin bound to avidin followed by detection of the enzymatic activity using a chromogenic substrate.

Alternatively, biotinylated antibodies may be incubated with a streptavidin-flurochrome.

Cell-Sorting Techniques

The ability to recognise dopaminergic cells or dopaminergic precursors with antibodies allows not only for the identification and quantification of these cells in tissue samples, but also for their separation and enrichment in suspension. This can be achieved by a number of cell-sorting techniques by which cells are physically separated by reference to a property associated with the cell-antibody complex, or a label attached to the antibody. This label may be a magnetic particle or a fluorescent molecule. The antibodies may be cross-linked such that they form aggregates of multiple cells, which are separable by their density. Alternatively the antibodies may be attached to a stationary matrix, to which the desired cells adhere.

Various methods of separating antibody-bound cells from unbound cells are known. For example, the antibody bound to the cell (or an anti-isotype antibody) can be labelled and then the cells separated by a mechanical cell sorter that detects the presence of the label. Fluorescence-activated cell sorters are well known in the art. In one embodiment, the anti-DA-protein antibody is attached to a solid support. Various solid supports are known to those of skill in the art, including, but not limited to, agarose beads, polystyrene beads, hollow fiber membranes, polymers, and plastic petri dishes. Cells that are bound by the antibody can be removed from the cell suspension by simply physically separating the solid support from the cell suspension. Preferred protocols, however, will be described.

Super paramagnetic nanoparticles may be used for cell separations. The microparticles are coated with a monoclonal antibody for a cell-surface antigen. The antibody-tagged, super paramagnetic microparticles are then incubated with a solution containing the cells of interest. The microparticles bind to the surfaces of the desired cells, and these cells can then be collected in a magnetic field.

Selective cytophoresis can be used to produce a cell suspension from mammalian brain containing dopaminergic neurons. The cell suspension is allowed to physically contact, for example, a solid phase-linked monoclonal antibody that recognizes an antigen on the desired cells. The solid-phase linking can comprise, for instance, adsorbing the antibodies to a plastic, nitrocellulose, or other surface. The antibodies can also be adsorbed on to the walls of the large pores (sufficiently large to permit flow-through of cells) of a hollow fiber membrane. Alternatively, the antibodies can be covalenty linked to a surface or bead, such as Pharmacia Sepharose 6MB macrobeads. The exact conditions and duration of incubation for the solid phase-linked antibodies with the CNS cell suspension will depend upon several factors specific to the system employed. The selection of appropriate conditions, however, is well within the skill of the art.

The unbound cells are then eluted or washed away with physiologic buffer after allowing sufficient time for the stem cells to be bound. The unbound cells can be recovered and used for other purposes or discarded after appropriate testing has been done to ensure that the desired separation had been achieved. The bound cells are then separated from the solid phase by any appropriate method, depending mainly upon the nature of the solid phase and the antibody. For example, bound cells can be eluted from a plastic petri dish by vigorous agitation. Alternatively, bound cells can be eluted by enzymatically “nicking” or digesting an enzyme-sensitive “spacer” sequence between the solid phase and the antibody. Spacers bound to agarose beads are commercially available from, for example, Pharmacia.

The eluted, enriched fraction of cells may then be washed with a buffer by centrifugation and either said enriched fraction or the unbound fraction may be cryopreserved in a viable state for later use according to conventional technology or introduced into the transplant recipient.

The term ‘enriched’ is used to describe a population of cells in which the proportion of one particular cell type or the proportion of a number of particular cell types is increased when compared with the untreated population. The term ‘diminished’ is used to describe a population of cells in which the proportion of one particular cell type or the proportion of a number of particular cell types is decreased when compared with the untreated population.

The Enriched Compositions of the Invention

The present invention further relates to a composition comprising a population containing cells originating from mammalian neural cells, wherein the percentage of cells expressing a DA-gene is at least 10%. Preferably, the percentage of cells expressing a DA-gene is at least 20%, more preferably at least 30%, more preferably at least 40%, more preferably at least 50%, more preferably at least 60%, more preferably at least 70%, more preferably at least 80% and most preferably at least 90%.

Also, the present invention relates to a composition comprising a population of cells obtainable by a method comprising the steps of:

a) combining a starting population containing cells originating from mammalian neural cells with antibodies which bind specifically to an extraceLlular epitope of a DA-protein to produce a first cell mixture,

b) removing unbound antibodies from the first cell mixture to produce a second cell mixture, and

c) separating cells comprising said antibodies from the second cell mixture to produce a cell population enriched in cells expressing said DA-gene and a cell population diminished in cells expressing said DA-gene.

In addition, the present invention relates to a cell expressing a gene of the invention obtained by the method of the invention, wherein the cell has been subjected to a genetic modification. Preferably, the gene is selected from the group consisting of KIAA1145, SLC10A4, SLC2A13, LRRC3B and FLJ45455.

Uses of the Cell Composition of the Invention

The present invention relates to the use of the composition of the invention or the cells of the invention for transplantation, for drug screening and for gene expression analysis. Furthermore, the present invention relates to the use of the composition of the invention or the cell of the invention as an immunogen for generation of antibodies.

Also, the present invention relates to an implantable encapsulated device comprising the composition of the invention or the cell of the invention.

In addition, the present invention relates to a method of identifying cells expressing a DA-gene in a population containing cells originating from mammalian neural cells comprising contacting the cells with a LabelLed antibody to a DA-protein of the invention and detecting the labelling.

As indicated above, one application for antibodies is the isolation of an enriched source of dopaminergic neurons for transplantation into patients with Parkinson's disease.

The present invention contemplates the use of methods employing an antibody to separate dopaminergic neurons or dopaminergic progenitor cells from other neural cells. Generally, a cell suspension prepared from human CNS tissue (e.g. from human fetal brain) is brought into contact with an antibody. Cells that have been bound by antibody are then separated from unbound cells by any means known to those skilled in the art. The CNS tissue may be taken from any part of the brain or spinal cord and may be selected by dissection of particular regions, which contain particular cell types. For instance the ventral mesencephalon may be selected to provide dopaminergic neurons and the substantia nigra pars compacta is particularly rich in dopaminergic neurons. The developing ventral mesencephalon may be particularly suitable for the enrichment of immature dopaminergic neurons and their commited progenitors.

In a further embodiment, the invention provides cell populations useful in methods of ex vivo gene therapy. Expression vectors may be introduced into and expressed in these cells, or their genome may be modified by homologous or non-homologous recombination by methods known in the art. In this way, diseases may be treated, which are related to the lack of secreted proteins including, but not limited to hormones, enzymes, and growth factors. Inducible expression of a gene of interest under the control of an appropriate regulatory initiation region will allow production (and secretion) of the protein in a fashion similar to that in the cell that normally produces the protein in nature. Antibodies that label the populations of Dopaminergic neurons or precursors are extremely useful in drug screening, gene discovery and for transplantation purposes because they allow the enrichment of populations of e.g. dopaminergic neurons or their progenitors in a single step. Cells recovered with antibody derived from different stages in their development could be used in studies on the mechanisms of action of cells, factors, and genes that regulate dopaminergic cell proliferation and differentiation. Furthermore, dopaminergic neurons from normal and pathological brain tissue may be recovered using antibodies and compared.

The above cell populations containing enriched cells can be used in therapeutic methods such as cell transplantation, as well as other methods that are readily apparent to those skilled in the art. Other uses envisaged for these cells are for drug screening, antibody production and gene discovery.

In another embodiment, the antibodies can be used to isolate enriched cells, which can be used in various protocols of genetic therapy.

Methods for Differentiation of Dopaminergic Neurons

Developing dopaminergic (DAergic) neurons originating from aborted human embryos have been implanted in the brains of patients with Parkinson's disease (PD) and in some cases successfully restored function. However, there are not sufficient numbers of cells available to allow this to develop into a widely used therapy. The limited availability of tissue from embryos may be circumvented by the use of cell lines that can be expanded in vitro for banking and then differentiated into DAergic neurons just prior to implantation into patients. Today, there are four main sources for such cell lines with a future potential for banking and cell therapy for PD: Human embryonic stem cells, human neural stem cells, human genetically immortalized stem/progenitor cells, and human adult-derived non-neural stem cells such as bone marrow-derived stem cells. Currently, it is not possible to utilize these cell sources therapeutically for PD. The primary reasons are that it has not been possible to effectively differentiate these different cells into DAergic neurons and stability of phenotypic expression has been variable.

Different sources of cells may be used for generation of dopaminergic neurons. These include embryonic neural stem cells, preferably cells isolated from the mesencephalon of embryos. Embryonic neural stem and progenitor cells are obtained from embryonic neural tissue and are capable of self-renewing and generating neurons and glia. Research over the past decade has shown that it is possible to select, epigenetically manipulate, and genetically engineer cells in culture prior to intracerebral transplantation. Neural stem cells have been isolated from various parts of the brain, such as the midbrain and forebrain. These cells can be grown as free-floating cell cultures, in so called neurospheres, where they will expand in number. These cells appear to retain their multipotency and the ability to develop along different progenitor, neuronal, and glial lineages under specific culture conditions. One preferred type of neural precursor cells include cells expressing the markers, GFAP and Nestin (U.S. Pat. No. 6,878,543, incorporated by reference).

These may be differentiated into dopaminergic neurons using both epigenetic factors using cytokines and EPO (Studer, L., et al., J Neurosci, 2000. 20(19): p. 7377-83; Storch, A., et al., Exp Neurol, 2001. 170(2): p. 317-25.) and genetic factors using Nurr1 (Kim, J.Y., et al., J Neurochem, 2003. 85(6): p. 1443-54). Another source of cells for dopaminergic differentiation include immortalised neural cell lines, which are typically established by transduction with a retrovirus or lentivirus coding for an oncogene, such as V-myc. By constitutively expressing oncogenes such as v-myc, cell lines proliferate indefinitely in culture, although they still depend on mitogens such as basic fibroblast growth factor (bFGF), epidermal growth factor or serum to divide. In the absence of mitogens, they exit the cell cycle and differentiate. Differentiation into a dopaminergic phenotype requires special culture conditions (US 2004/0247571). Another way to generate neural progenitor cell lines for PD is to overexpress Nurr1 in existing neural stem cell lines. Cells resembling midbrain DAergic neurons have been obtained from an immortalized multipotent neural stem cell line by overexpressing Nurr1, fibroblast growth factor-8 (FGF-8), and sonic hedgehog (Shh) (Kim, T. E., et al., Biochem Biophys Res Commun, 2003. 305(4): p. 1040-8). Shh and FGF-8 are known to direct the differentiation of mesencephalic DA neurons during the development (Ye, W., et al., Cell, 1998. 93(5): p. 755-66).

Embryonal stem cells are another source of cells for generation of dopaminergic neurons for transplantation. Concerning a directed differentiation of human ES cells into DAergic neurons, two studies have been published so far (Park, S., et al., Neurosci Lett, 2004. 359(1-2): p. 99-103; Perrier, A.L., et al., Proc Natl Acad Sci U S A, 2004. 101(34): p. 12543-8).

The first method involves the formation of EBs followed by selection of neuronal stem cells by incubation in serum-free insulin/transferrin/selenium/fibronectin medium and expansion of neural stem cells in the presence of bFGF (Park et al, op cit). In the final step, DAergic neurons are enriched by removal of bFGF and addition of transforming growth factor-α (TGF-α). Following 21 days in the presence of TGF-A, −15% of the differentiated human ES cells in culture express TH and release DA.

The second method (Perrier et al, op cit) is an adaptation of a previously published method for DAergic neuronal differentiation of mouse ES cells. The first step of the 50 days differentiation procedure is an initial neural induction on stromal cells, followed by neural stem cell expansion and differentiation by sequential exposure to Shh, FGF-8, BDNF, glial cell line-derived neurotrophic factor (GDNF), dibutyryl cAMP, AA and transforming growth factor-β3 (TGF-β3). This protocol has been tested on three different human ES cell lines. Interestingly, the proportions of cells that differentiate into TH-immunopositive neurons, expressed as a percentage of the β-III-tubulin expressing cells, are both relatively high and similar (−64%, −70% and −79%).

Differentiation of embryonal stem cells into dopaminergic neurons may also include the use of genetic factors, notably Nurr1, as opposed to soluble protein factors or a combination of both methods.

Further sources of cells for generation of dopaminergic neurons include somatic stem cells such as adult bone marrow cells and ubelical cord blood cells.

The DA-genes and DA-proteins described herein may be used in combination with any of the methods known in the art for differentiation of dopaminergic neurons. The secreted growth factors presented in Tables 5 and 6 and the preferred growth factors from these tables described herein may be used as epigenetic factors or genes encoding these secreted growth factors may be used for transfecting or transducing a composition of cells as described above. All other DA-genes described herein may be used for genetically modifying cells capable of differentiating into dopaminergic neurons.

Methods for expansion and differentiation of neural stem and/or precursor cells The DA-genes in general and the growth factors of Tables 5 and 6 in particular, moree preferably the growth factors of Table 6, may be used for expansion and/or differentiation of mammalian cell cultures, in particular for expansion and/or differentiation of neural stem and precursor cells. Cells that may be used for this aspect of the invention include the sources of cells described above for differentiation of Dopaminergic neurons.

Screening Assays

The invention provides a method (also referred to herein as a “screening assay”) for identifying modulators, i.e., candidate or test compounds or agents (e.g., peptides, peptidomimetics, small molecules or other drugs) that bind to DA-proteins or have a stimulatory or inhibitory effect on, e.g., DA-protein expression or DA-protein activity.

In one embodiment, the invention provides assays for screening candidate or test compounds which bind to or modulate the activity of the membrane-bound form of a DA-protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic Library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, poLypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37: 1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; FeLici, 1991. J. Mol. Biol. 222: 301-310; Ladner, U.S. Pat. No. 5,233,409.).

In one embodiment, an assay is a cell-based assay in which a cell which expresses a membrane-bound form of DA-protein, or a biologically-active portion thereof, on the cell surface is contacted with a test compound and the ability of the test compound to bind to a DA-protein determined. The cell, for example, can of mammalian origin or a yeast cell. Determining the ability of the test compound to bind to the DA-protein can be accomplished, for example, by coupling the test compound with a radioisotope or enzymatic label such that binding of the test compound to the DA-protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of DA-protein, or a biologically-active portion thereof, on the cell surface with a known compound which binds DA-protein to form an assay mixture, contacting the assay mixture with a test compound, and determining the ability of the test compound to interact with a DA-protein, wherein determining the ability of the test compound to interact with a DA-protein comprises determining the ability of the test compound to preferentially bind to DA-protein or a biologically-active portion thereof as compared to the known compound.

In another embodiment, an assay is a cell-based assay comprising contacting a cell expressing a membrane-bound form of DA-protein, or a biologically-active portion thereof, on the cell surface with a test compound and determining the ability of the test compound to modulate (e.g., stimulate or inhibit) the activity of the DA-protein or biologically-active portion thereof. Determining the ability of the test compound to modulate the activity of a DA-protein or a biologically-active portion thereof can be accomplished, for example, by determining the ability of the DA-protein to bind to or interact with a DA-protein target molecule. As used herein, a “target molecule” is a molecule with which a DA-protein binds or interacts in nature, for example, a molecule on the surface of a cell which expresses a DA-protein interacting protein, a molecule on the surface of a second cell, a molecule in the extracellular milieu, a molecule associated with the internal surface of a cell membrane or a cytoplasmic molecule. A DA-protein target molecule can be a non-DA-protein molecule or a DA-protein or polypeptide of the invention. In one embodiment, a DA-protein target molecule is a component of a signal transduction pathway that facilitates transduction of an extracellular signal (e.g. a signal generated by binding of a compound to a membrane-bound DA-protein) through the cell membrane and into the cell. The target, for example, can be a second intercellular protein that has catalytic activity or a protein that facilitates the association of downstream signaling molecules with a DA-protein.

Determining the ability of the DA-protein to bind to or interact with a DA-protein target molecule can be accomplished by one of the methods described above for determining direct binding. In one embodiment, determining the ability of the DA-protein to bind to or interact with a DA-protein target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising a DA-protein -responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting a DA-protein or biologically-active portion thereof with a test compound and determining the ability of the test compound to bind to the DA-protein or biologically-active portion thereof. Binding of the test compound to the DA-protein can be determined either directly or indirectly as described above. In one such embodiment, the assay comprises contacting the DA-protein or biologically-active portion thereof with a known compound which binds DA-protein to form an assay mixture, contacting the assay mixture with a test compound, and determining the ability of the test compound to interact with a DA-protein, wherein determining the ability of the test compound to interact with a DA-protein comprises determining the ability of the test compound to preferentially bind to DA-protein or biologically-active portion thereof as compared to the known compound.

In still another embodiment, an assay is a cell-free assay comprising contacting DA-protein or biologically-active portion thereof with a test compound and determining the ability of the test compound to modulate (e.g. stimulate or inhibit) the activity of the DA-protein or biologically-active portion thereof. Determining the ability of the test compound to modulate the activity of DA-protein can be accomplished, for example, by determining the ability of the DA-protein to bind to a DA-protein molecule by one of the methods described above for determining direct binding. In an alternative embodiment, determining the ability of the test compound to modulate the activity of DA-protein can be accomplished by determining the ability of the DA-protein further modulate a DA-protein target molecule. For example, the catalytic/enzymatic activity of the target molecule on an appropriate substrate can be determined as described, supra.

In yet another embodiment, the cell-free assay comprises contacting the DA-protein or biologically-active portion thereof with a known compound which binds DA-protein to form an assay mixture, contacting the assay mixture with a test compound, and determining the ability of the test compound to interact with a DA-protein, wherein determining the ability of the test compound to interact with a DA-protein comprises determining the ability of the DA-protein to preferentially bind to or modulate the activity of a DA-protein target molecule.

The cell-free assays of the invention are amenable to use of both the soluble form or the membrane-bound form of DA-protein. In the case of cell-free assays comprising the membrane-bound form of DA-protein, it may be desirable to utilize a solubilizing agent such that the membrane-bound form of DA-protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl--N,N-dimethyl-3-ammonio-1-propane sulfonate, 3-(3-cholamidopropyl) dimethylamminiol-1 -propane sulfonate (CHAPS), or 3-(3-cholamidopropyl)dimethylamminiol-2-hydroxy-1 -propane sulfonate (CHAPSO).

In more than one embodiment of the above assay methods of the invention, it may be desirable to immobilize either a DA-protein or its target molecule to facilitate separation of complexed from uncomplexed forms of one or both of the proteins, as well as to accommodate automation of the assay. Binding of a test compound to a DA-protein, or interaction of a DA-protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-DA-protein fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or DA-protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of DA-protein binding or activity determined using standard techniques.

Other techniques for immobilizing proteins on matrices can also be used in the screening assays of the invention. For example, either the DA-protein or its target molecule can be immobilized utilizing conjugation of biotin and streptavidin. Biotinylated DA-protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with a DA-protein or target molecules, but which do not interfere with binding of the DA-protein to its target molecule, can be derivatized to the wells of the plate, and unbound target or DA-protein trapped in the wells by antibody conjugation. Methods for detecting such complexes, in addition to those described above for the GST-immobilized complexes, include immunodetection of complexes using antibodies reactive with the DA-protein or target molecule, as well as enzyme-linked assays that rely on detecting an enzymatic activity associated with the DA-protein or target molecule.

In another embodiment, modulators of DA-protein expression are identified in a method wherein a cell is contacted with a candidate compound and the expression of DA-gene mRNA or DA-protein in the cell is determined. The level of expression of DA-gene mRNA or DA-protein in the presence of the candidate compound is compared to the level of expression of DA-gene mRNA or DA-protein in the absence of the candidate compound. The candidate compound can then be identified as a modulator of DA-gene mRNA or DA-protein expression based upon this comparison. For example, when expression of DA-gene mRNA or DA-protein is greater (i.e., statistically significantly greater) in the presence of the candidate compound than in its absence, the candidate compound is identified as a stimulator of DA-gene mRNA or DA-protein expression. Alternatively, when expression of DA-gene mRNA or DA-protein is less (statistically significantly less) in the presence of the candidate compound than in its absence, the candidate compound is identified as an inhibitor of DA-gene mRNA or DA-protein expression. The level of DA-gene mRNA or DA-protein expression in the cells can be determined by methods described herein for detecting DA-gene mRNA or DA-protein.

In yet another aspect of the invention, the DA-proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with DA-protein (“DA-protein-binding proteins” or “DA-protein-bp”) and modulate DA-protein activity. Such DA-protein-binding proteins are also likely to be involved in the propagation of signals by the DA-proteins as, for example, upstream or downstream elements of the DA-gene pathway.

The two-hybrid system is based on the modular nature of most transcription factors, which consist of separable DNA-binding and activation domains. Briefly, the assay utilizes two different DNA constructs. In one construct, the gene that codes for DA-protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a DA-protein-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with DA-gene.

The invention further pertains to novel agents identified by the aforementioned screening assays and uses thereof for treatments as described herein.

Monitoring of Effects During Clinical Trials

Monitoring the influence of agents (e.g., drugs, compounds) on the expression or activity of DA-gene (e.g., the ability to modulate aberrant cell proliferation and/or differentiation) can be applied not only in basic drug screening, but also in clinical trials. For example, the effectiveness of an agent determined by a screening assay as described herein to increase DA-gene gene expression, protein levels, or upregulate DA-gene activity, can be monitored in clinical trails of subjects exhibiting decreased DA-gene gene expression, protein levels, or downregulated DA-gene activity. Alternatively, the effectiveness of an agent determined by a screening assay to decrease DA-gene expression, protein levels, or downregulate DA-gene activity, can be monitored in clinical trails of subjects exhibiting increased DA-gene expression, protein levels, or upregulated DA-gene activity. In such clinical trials, the expression or activity of DA-gene and, preferably, other genes that have been implicated in, Dopaminergic phenotype or Parkinson's Disease can be used as a “read out” or markers of the Dopaminergic neurons. For transporter proteins, a fluorgenic substrate that can be transported across the cellular membrane can be used in vivo to label cells expressing the transporter and the amount of such labelled cells can be detected by, e.g. PET scanning, as is done e.g. with fluorodopa.

By way of example, and not of limitation, genes, including DA-gene, that are modulated in cells by treatment with an agent (e.g., compound, drug or small molecule) that modulates DA-gene activity (e.g., identified in a screening assay as described herein) can be identified. Thus, to study the effect of agents on cellular proliferation disorders, for example, in a clinical trial, cells can be isolated and RNA prepared and analyzed for the levels of expression of DA-gene and other genes implicated in the disorder. The levels of gene expression (i.e., a gene expression pattern) can be quantified by Northern blot analysis or RT-PCR, as described herein, or alternatively by measuring the amount of protein produced, or by measuring the levels of activity of DA-gene or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of a DA-protein, or DA-gene mRNA in the preadministration sample; (iii) obtaining one or more post-administration samples from the subject; (iv) detecting the level of expression or activity of the DA-protein or DA-gene mRNA in the post-administration samples; (v) comparing the level of expression or activity of the DA-protein, DA-gene mRNA in the pre-administration sample with the DA-protein, DA-gene mRNA in the post administration sample or samples; and (vi) altering the administration of the agent to the subject accordingly. For example, increased administration of the agent may be desirable to increase the expression or activity of DA-gene to higher levels than detected, i.e., to increase the effectiveness of the agent. Alternatively, decreased administration of the agent may be desirable to decrease expression or activity of DA-gene to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant DA-gene expression or activity. The disorders neurodegenerative disorders in general and Parkinson's Disease in particular. These methods of treatment will be discussed more fully, below.

Diseases and Disorders

Diseases and disorders that are characterized by increased (relative to a subject not suffering from the disease or disorder) levels or biological activity may be treated with therapeutics that antagonize (i.e., reduce or inhibit) activity. Therapeutics that antagonize activity may be administered in a therapeutic or prophylactic manner. Therapeutics that may be utilized include, but are not limited to: (i) a DA-peptide, or analogs, derivatives, or fragments thereof; (ii) antibodies to a DA-peptide; (iii) nucleic acids encoding a DA-peptide; (iv) administration of antisense nucleic acid and nucleic acids that are “dysfunctional” (i.e., due to a heterologous insertion within the coding sequences of coding sequences to a DA-peptide) that are utilized to “knockout” endogenous function of an aforementioned peptide by homologous recombination (see, e.g., Capecchi, 1989. Science 244: 1288-1292); or (v) modulators ( i.e., inhibitors, agonists and antagonists, including additional peptide mimetic of the invention or antibodies specific to a DA-peptide of the invention) that alter the interaction between a DA-peptide and its binding partner.

Diseases and disorders that are characterized by decreased (relative to a subject not suffering from the disease or disorder) levels or biological activity may be treated with therapeutics that increase (i.e., are agonists to) activity. Therapeutics that upregulate activity may be administered in a therapeutic or prophylactic manner. Therapeutics that may be utilized include, but are not limited to, a DA-peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, Parkinson's Disease, which may be associated with an aberrant DA-gene expression or activity, by administering to the subject an agent that modulates DA-gene expression or at least one DA-gene activity. Subjects at risk for Parkinson's disease that is caused or contributed to by aberrant DA-gene expression or activity can be identified by diagnostic methods known in the art. Administration of a prophylactic agent can occur prior to the manifestation of symptoms characteristic of the Parkinson's Disease, such that the disease is prevented or, alternatively, delayed in its progression. Depending upon the type of DA-gene aberrancy, for example, a DA-gene agonist or DA-gene antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating DA-gene expression or activity for therapeutic purposes. The modulatory method of the invention involves contacting a cell with an agent that modulates one or more of the activities of DA-protein activity associated with the cell. An agent that modulates DA-protein activity can be an agent as described herein, such as a nucleic acid or a protein, a naturally-occurring cognate ligand of a DA-protein, a peptide, a DA-protein peptidomimetic, or other small molecule. In one embodiment, the agent stimulates one or more DA-protein activities. Examples of such stimulatory agents include active DA-protein and a nucleic acid molecule encoding a DA-protein that has been introduced into the cell. In another embodiment, the agent inhibits one or more DA-protein activities. Examples of such inhibitory agents include antisense DA-gene nucleic acid molecules and anti-DA-protein antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of a DA-protein or nucleic acid molecule. In one embodiment, the method involves administering an agent (e.g., an agent identified by a screening assay described herein), or combination of agents that modulates (e.g., up-regulates or down-regulates) DA-gene expression or activity. In another embodiment, the method involves administering a DA-protein or nucleic acid molecule as therapy to compensate for reduced or aberrant DA-gene expression or activity.

Stimulation of DA-gene activity is desirable in situations in which DA-gene is abnormally downregulated and/or in which increased DA-gene activity is likely to have a beneficial effect.

Prophylactic and Therapeutic Uses of the Compositions of the Invention

The DA-gene nucleic acids and proteins of the invention are useful in potential prophylactic and therapeutic applications implicated in neurodegenerative disorders including Parkinson's Disorder.

As an example, a cDNA encoding the DA-protein of the invention may be useful in gene therapy, and the protein may be useful when administered to a subject in need thereof. By way of non-limiting example, the compositions of the invention will have efficacy for treatment of patients suffering from: neurodegenerative disorders, in particular Parkinson's Disorder.

Both the novel nucleic acid encoding the DA-protein, and the DA-protein of the invention, or fragments thereof, may also be useful in diagnostic applications, wherein the presence or amount of the nucleic acid or the protein are to be assessed. These materials are further useful in the generation of antibodies, which immunospecifically-bind to the novel substances of the invention for use in therapeutic or diagnostic methods.

The invention will be further described in the following examples, which do not limit the scope of the invention described in the claims.

Pharmaceutical Preparations for Gene Therapy

To form a DA-gene composition for gene therapy use in the invention, DA-gene encoding expression viral vectors may be placed into a pharmaceutically acceptable suspension, solution or emulsion. Suitable mediums include saline and liposomal preparations.

More specifically, pharmaceutically acceptable carriers may include sterile aqueous of non-aqueous solutions, suspensions, and emulsions. Examples of nonaqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils.

Intravenous vehicles include fluid and nutrient replenishers, electrolyte replenishers (such as those based on Ringer's dextrose), and the like.

Preservatives and other additives may also be present such as, for example, antimicrobials, antioxidants, chelating agents, and inert gases and the like. Further, a composition of DA-gene transgenes may be lyophilized using means well known in the art, for subsequent reconstitution and use according to the invention.

A colloidal dispersion system may also be used for targeted gene delivery. Colloidal dispersion systems include macromolecule complexes, nanocapsules, microspheres, beads, and lipid-based systems including oil-in-water emulsions, micelles, mixed micelles, and liposoms. Liposomes are artificial membrane vesicles which are useful as delivery vehicles in vitro and in vivo. It has been shown that large unilamellar vesicles (LUV), which range in size from 0.2-4.0 μm can encapsulate a substantial percentage of an aqueous buffer containing large macro molecules. RNA, DNA and intact virions can be encapsulated within the aqueous interior and be delivered to cells in a biologically active form (Fraley, et al., Trends Biochem. Sci., 6: 77,1981). In addition to mammalian cells, liposomes have been used for delivery of operatively encoding transgenes in plant, yeast and bacterial cells. In order for a liposome to be an efficient gene transfer vehicle, the following characteristics should be present: (1) encapsulation of the genes encoding the DA-gene at high efficiency while not compromising their biological activity; (2) preferential and substantial binding to a target cell in comparison to non-target cells; (3) delivery of the aqueous contents of the vesicle to the target cell cytoplasm at high efficiency; and (4) accurate and effective expression of genetic information (Mannino, et al., Biotechniques, 6: 682,1988).

The composition of the liposome is usually a combination of phospholipids, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations.

Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Particularly useful are diacylphosphatidylglycerols, where the lipid moiety contains from 14-18 carbon atoms, particularly from 16-18 carbon atoms, and is saturated. Illustrative phospholipids include egg phosphatidylcholine, dipalmitoylphosphatidylcholine and distearoylphosphatidylcholine.

The targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticulo-endothelial system (RES) in organs which contain sinusoidal capillaries.

Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization.

The surface of the targeted gene delivery system may be modified in a variety of ways. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand.

A further example of a delivery system includes transplantation into the therapeutic area of a composition of packaging cells capable of producing vector particles as described in the present invention. Methods for encapsulation and transplantation of such cells are known in the art, in particular from WO 97/44065 (Cytotherapeutics). By selecting a packaging cell line capable of producing lentiviral particles, transduction of non-dividing cells in the therapeutic area is obtained. By using retroviral particles capable of transducing only dividing cells, transduction is restricted to newly generated cells in the therapeutic area.

Dosing Requirements and Delivery Protocol for Gene Therapy

An important parameter is the dosage of DA-gene therapy vector to be delivered into the target tissue. For viral vectors, the concentration may be defined by the number of transducing units/ml. Optimally, for delivery using a viral expression vector, each unit dosage will comprise 2.5 to 25 μL of a composition, wherein the composition includes a viral expression vector in a pharmaceutically acceptable fluid and provides from 108 up to 10¹² DA-gene transducing units per ml.

Importantly, specific in vivo gene delivery sites are selected so as to cluster in an area of loss, damage, or dysfunction of neural cells, glial cells, retinal cells, sensory cells, or stem cells. Such areas may be identified clinically using a number of known techniques, including magnetic resonance imaging (MRI) and biopsy. In humans, non-invasive, in vivo imaging methods such as MRI will be preferred. Once areas of neuronal loss are identified, delivery sites are selected for stereotaxic distribution so each unit dosage of DA-gene is delivered into the brain at, or within 500 μm from, a targeted cell, and no more than about 10 mm from another delivery site.

Within a given target site, the vector system may transduce a target cell. The target cell may be a cell found in nervous tissue, such as a neuron, astrocyte, oligodendrocyte, microglia, stem cells, neural precursor cells, or ependymal cell.

The vector system is preferably administered by direct injection. Methods for injection into the brain are well known in the art (Bilang-Bleuet et al (1997) Proc. Acad. Nati. Sci. USA 94:8818-8823; Choi-Lundberg et al (1998) Exp. Neurol.154:261-275; Choi-Lundberg et al (1997) Science 275:838-841; and Mandel et al (1997) ) Proc. Acad. Nati. Sci. USA 94:14083-14088). Stereotaxic injections may be given.

As mentioned above, for transduction in tissues such as the brain, it is necessary to use very small volumes, so the viral preparation is concentrated by ultracentrifugation. The resulting preparation should have at least 10⁸ t.u./ml, preferably from 10⁸ to 10¹⁰ t.u./ml, more preferably at least 10⁹ t.u./ml. (The titer is expressed in transducing units per ml (t.u./ml) as described in example 7). It has been found that improved dispersion of transgene expression can be obtained by increasing the number of injection sites and decreasing the rate of injection (Horellou and Mallet (1997) as above). Usually between 1 and 10 injection sites are used, more commonly between 2 and 6. For a dose comprising 1-5×10⁹ t.u./ml, the rate of injection is commonly between 0.1 and 10 μl min, usually about 1 μl/min.

The virus composition is delivered to each delivery cell site in the target tissue by microinjection, infusion, scrape loading, electroporation or other means suitable to directly deliver the composition directly into the delivery site tissue through a surgical incision. The delivery is accomplished slowly, such as over a period of about 5-10 minutes (depending on the total volume of virus composition to be delivered).

Viral Vectors

Broadly, gene therapy seeks to transfer new genetic material to the cells of a patient with resulting therapeutic benefit to the patient. Such benefits include treatment or prophylaxis of a broad range of diseases, disorders and other conditions.

Ex vivo gene therapy approaches involve modification of isolated cells (including but not limited to stem cells, neural and glial precursor cells, and foetal stem cells), which are then infused, grafted or otherwise transplanted into the patient. See, e.g., U.S. Pat. Nos. 4,868,116, 5,399,346 and 5,460,959. In vivo gene therapy seeks to directly target host patient tissue in vivo.

Viruses useful as gene transfer vectors include papovavirus, adenovirus, vaccinia virus, adeno-associated virus, herpesvirus, and retroviruses. Suitable retroviruses include the group consisting of HIV, SIV, FIV, EIAV, MOMLV.

Preferred viruses for treatment of disorders of the nervous system are lentiviruses and adeno-associated viruses. Both types of viruses can integrate into the genome without cell divisions, and both types have been tested in pre-clinical animal studies for indiations of the nervous system, in particular the central nervous system.

Methods for preparation of AAV are described in the art, e.g. U.S. Pat. No. 5,677,158. U.S. Pat. No. 6,309,634 and U.S. Pat. No. 6,683,058 describe examples of delivery of AAV to the central nervous system.

Preferably, a lentivirus vector is a replication-defective lentivirus particle. Such a lentivirus particle can be produced from a lentiviral vector comprising a 5′ lentiviral LTR, a tRNA binding site, a packaging signal, a promoter operably linked to a polynucleotide signal encoding said fusion protein, an origin of second strand DNA synthesis and a 3′ lentiviral LTR. Methods for preparation and in vivo administration of lentivirus to neural cells are described in US 20020037281 (Methods for transducing neural cells using lentiviral vectors).

Retroviral vectors are the vectors most commonly used in human clinical trials, since they carry 7-8 kb and since they have the ability to infect cells and have their genetic material stably integrated into the host cell with high efficiency. See, e.g., WO 95/30761; WO 95/24929. Oncovirinae require at least one round of target cell proliferation for transfer and integration of exogenous nucleic acid sequences into the patient. Retroviral vectors integrate randomly into the patients genome. Retroviruses can be used to target stem cells of the nervous system as very few cell divisions take place in other cells of the nervous system (in particular the CNS).

Three classes of retroviral particles have been described; ecotropic, which can infect murine cells efficiently, and amphotropic, which can infect cells of many species. The third class includes xenotrophic retrovirus which can infect cells of another species than the species which produced the virus. Their ability to integrate only into the genome of dividing cells has made retroviruses attractive for marking cell lineages in developmental studies and for delivering therapeutic or suicide genes to cancers or tumours.

For use in human patients, the retroviral vectors preferably are replication defective. This prevents further generation of infectious retroviral particles in the target tissue—instead the replication defective vector becomes a “captive” transgene stable incorporated into the target cell genome. Typically in replication defective vectors, the gag, env, and pot genes have been deleted (along with most of the rest of the viral genome). Heterologous DNA is inserted in place of the deleted viral genes. The heterologous genes may be under the control of the endogenous heterologous promoter, another heterologous promoter active in the target cell, or the retroviral 5′ LTR (the viral LTR is active in diverse tissues). Typically, retroviral vectors have a transgene capacity of about 7-8 kb.

Replication defective retroviral vectors require provision of the viral proteins necessary for replication and assembly in trans, from, e.g., engineered packaging cell lines. It is important that the packaging cells do not release replication competent virus and/or helper virus. This has been achieved by expressing viral proteins from RNAs lacking the y signal, and expressing the gag/pol genes and the env gene from separate transcriptional units. In addition, in some 2nd and 3rd generation retroviruses, the 5′ LTR's have been replaced with non-viral promoters controlling the expression of these genes, and the 3′ promoter has been minimised to contain only the proximal promoter. These designs minimize the possibility of recombination leading to production of replication competent vectors, or helper viruses.

Expression Vectors

Construction of vectors for recombinant expression of DA-gene polypeptides for use in the invention may be accomplished using conventional techniques, which do not require detailed explanation to one of ordinary skill in the art. For review, however, those of ordinary skill may wish to consult Maniatis et al., in Molecular Cloning: A Laboratory Manual, Cold Spring Harbor Laboratory, (NY 1982). Expression vectors may be used for generating producer cells for recombinant production of DA-gene polypeptides for medical use, and for generating therapeutic cells secreting DA-gene polypeptides for naked or encapsulated therapy.

Briefly, construction of recombinant expression vectors employs standard ligation techniques. For analysis to confirm correct sequences in vectors constructed, the genes are sequenced using, for example, the method of Messing, et al., (Nucleic Acids Res., 9: 309-, 1981), the method of Maxam, et al., (Methods in Enzymology, 65: 499, 1980), or other suitable methods which will be known to those skilled in the art.

Size separation of cleaved fragments is performed using conventional gel electrophoresis as described, for example, by Maniatis, et al., (Molecular Cloning, pp. 133-134,1982).

For generation of efficient expression vectors, these should contain regulatory sequences necessary for expression of the encoded gene in the correct reading frame.

Expression of a gene is controlled at the transcription, translation or post-translation levels. Transcription initiation is an early and critical event in gene expression. This depends on the promoter and enhancer sequences and is influenced by specific cellular factors that interact with these sequences. The transcriptional unit of many genes consists of the promoter and in some cases enhancer or regulator elements (Banerji et al., Cell 27: 299 (1981); Corden et al., Science 209: 1406 (1980); and Breathnach and Chambon, Ann. Rev. Biochem. 50: 349 (1981)). For retroviruses, control elements involved in the replication of the retroviral genome reside in the long terminal repeat (LTR) (Weiss et al., eds., The molecular biology of tumor viruses: RNA tumor viruses, Cold Spring Harbor Laboratory, (NY 1982)). Moloney murine leukemia virus (MLV) and Rous sarcoma virus (RSV) LTRs contain promoter and enhancer sequences (Jolly et al., Nucleic Acids Res. 11: 1855 (1983); Capecchi et al., in: Enhancer and eukaryotic gene expression, Gulzman and Shenk, eds., pp. 101-102, Cold Spring Harbor Laboratories (NY 1991). Other potent promoters include those derived from cytomegalovirus (CMV) and other wild-type viral promoters.

Promoter and enhancer regions of a number of non-viral promoters have also been described (Schmidt et al., Nature 314: 285 (1985); Rossi and deCrombrugghe, Proc. Natl. Acad. Sci. USA 84: 5590-5594 (1987)). Methods for maintaining and increasing expression of transgenes in quiescent cells include the use of promoters including collagen type I (1 and 2) (Prockop and Kivirikko, N. Eng. J. Med. 311: 376 (1984); Smith and Niles, Biochem. 19: 1820 (1980); de Wet et al., J. Biol. Chem., 258: 14385 (1983)), SV40 and LTR promoters.

According to one embodiment of the invention, the promoter is a constitutive promoter selected from the group consisting of: ubiquitin promoter, CMV promoter, JeT promoter (U.S. Pat. No. 6,555,674), SV40 promoter, and Elongation Factor 1 alpha promoter (EF1-alpha).

Examples of inducible/repressible promoters include: Tet-On, Tet-Off, Rapamycin-inducible promoter, Mx1.

In addition to using viral and non-viral promoters to drive transgene expression, an enhancer sequence may be used to increase the level of transgene expression. Enhancers can increase the transcriptional activity not only of their native gene but also of some foreign genes (Armelor, Proc. Natl. Acad. Sci. USA 70 : 2702 (1973)). For example, in the present invention collagen enhancer sequences may be used with the collagen promoter 2 (I) to increase transgene expression. In addition, the enhancer element found in SV40 viruses may be used to increase transgene expression. This enhancer sequence consists of a 72 base pair repeat as described by Gruss et al., Proc. Natl. Acad. Sci. USA 78: 943 (1981); Benoist and Chambon, Nature 290: 304 (1981), and Fromm and Berg, J. Mol. Appl. Genetics, 1: 457 (1982), all of which are incorporated by reference herein. This repeat sequence can increase the transcription of many different viral and cellular genes when it is present in series with various promoters (Moreau et al., Nucleic Acids Res. 9: 6047 (1981).

Further expression enhancing sequences include but are not limited to Woodchuck hepatitis virus post-transcriptional regulation element (WPRE), SP163, CMV enhancer, and Chicken [beta]-globin insulator or other insulators.

Transgene expression may also be increased for long-term stable expression using cytokines to modulate promoter activity. Several cytokines have been reported to modulate the expression of transgene from collagen 2 (I) and LTR promoters (Chua et al., connective Tissue Res., 25: 161-170 (1990); Elias et al., Annals N. Y. Acad. Sci., 580 : 233-244 (1990)); Seliger et al., J. Immunol. 141: 2138-2144 (1988) and Seliger et al., J. Virology 62: 619-621 (1988)). For example, transforming growth factor (TGF), interleukin (IL)-l, and interferon (INF) down regulate the expression of transgenes driven by various promoters such as LTR. Tumor necrosis factor (TNF) and TGF 1 up regulate, and may be used to control, expression of transgenes driven by a promoter. Other cytokines that may prove useful include basic fibroblast growth factor (bFGF) and epidermal growth factor (EGF).

Biocompatible Capsules

Encapsulated cell therapy is based on the concept of isolating cells from the recipient host's immune system by surrounding the cells with a semipermeable biocompatible material before implantation within the host. The invention includes a device in which cells capable of expressing and secreting a DA-protein from Tables 5 or 6, preferably those from Table 6, are encapsulated in an immunoisolatory capsule. An “immunoisolatory capsule” means that the capsule, upon implantation into a recipient host, minimizes the deleterious effects of the host's immune system on the cells in the core of the device. Cells are immunoisolated from the host by enclosing them within implantable polymeric capsules formed by a microporous membrane. This approach prevents the cell-to cell contact between host and implanted tissues, eliminating antigen recognition through direct presentation. The membranes used can also be tailored to control the diffusion of molecules, such as antibody and complement, based on their molecular weight (Lysaght et al., 56 J. Cell Biochem. 196 (1996), Colton, 14 Trends Biotechnol. 158 (1996)). Using encapsulation techniques cells can be transplanted into a host without immune rejection, either with or without use of immunosuppressive drugs. Useful biocompatible polymer capsules usually contain a core that contains cells, either suspended in a liquid medium or immobilized within an immobilizing matrix, and a surrounding or peripheral region of permselective matrix or membrane (“jacket”) that does not contain isolated cells, that is biocompatible, and that is sufficient to protect cells in the core from detrimental immunological attack. Encapsulation hinders elements of the immune system from entering the capsule, thereby protecting the encapsulated cells from immune destruction. The semipermeable nature of the capsule membrane also permits the biologically active molecule of interest to easily diffuse from the capsule into the surrounding host tissue.

The capsule can be made from a biocompatible material. A “biocompatible material[ is a material that, after implantation in a host, does not elicit a detrimental host response sufficient to result in the rejection of the capsule or to render it inoperable, for example through degradation. The biocompatible material is relatively impermeable to large molecules, such as components of the host's immune system, but is permeable to small molecules, such as insulin, growth factors such as DA-protein from Tables 5 and 6, and nutrients, while allowing metabolic waste to be removed. A variety of biocompatible materials are suitable for delivery of growth factors by the composition of the invention. Numerous biocompatible materials are known, having various outer surface morphologies and other mechanical and structural characteristics. Preferably the capsule of this invention will be similar to those described in WO 92/19195 or WO 95/05452, incorporated by reference; or U.S. Pat. Nos. 5,639,275; 5,653,975; 4,892,538; 5,156,844; 5,283,187; or U.S. Pat. No. 5,550,050, incorporated by reference. Such capsules allow for the passage of metabolites, nutrients and therapeutic substances while minimizing the detrimental effects of the host immune system. Components of the biocompatible material may include a surrounding semipermeable membrane and the internal cell-supporting scaffolding. Preferably, the genetically altered cells are seeded onto the scaffolding, which is encapsulated by the permselective membrane. The filamentous cell-supporting scaffold may be made from any biocompatible material selected from the group consisting of acrylic, polyester, polyethylene, polypropylene polyacetonitrile, polyethylene teraphthalate, nylon, polyamides, polyurethanes, polybutester, silk, cotton, chitin, carbon, or biocompatible metals. Also, bonded fiber structures can be used for cell implantation (U.S. Pat. No. 5,512,600, incorporated by reference). Biodegradable polymers include those comprised of poly(lactic acid) PLA, poly(lactic-coglycolic acid) PLGA, and poly(glycolic acid) PGA and their equivalents. Foam scaffolds have been used to provide surfaces onto which transplanted cells may adhere (WO 98/05304, incorporated by reference). Woven mesh tubes have been used as vascular grafts (WO 99/52573, incorporated by reference). Additionally, the core can be composed of an immobilizing matrix formed from a hydrogel, which stabilizes the position of the cells. A hydrogel is a 3-dimensional network of cross-linked hydrophilic polymers in the form of a gel, substantially composed of water.

Various polymers and polymer blends can be used to manufacture the surrounding semipermeable membrane, including polyacrylates (including acrylic copolymers), polyvinylidenes, polyvinyl chloride copolymers, polyurethanes, polystyrenes, polyamides, cellulose acetates, cellulose nitrates, polysulfones (including polyether sulfones), polyphosphazenes, polyacrylonitriles, poly(acrylonitrile/covinyl chloride), as well as derivatives, copolymers and mixtures thereof. Preferably, the surrounding semipermeable membrane is a biocompatible semipermeable hollow fiber membrane. Such membranes, and methods of making them are disclosed by U.S. Pat. Nos. 5,284,761 and 5,158,881, incorporated by reference. The surrounding semipermeable membrane is formed from a polyether sulfone hollow fiber, such as those described by U.S. Pat. No. 4,976,859 or U.S. Pat. No. 4,968,733, incorporated by reference. An alternate surrounding semipermeable membrane material is poly(acrylonitrile/covinyl chloride).

The capsule can be any configuration appropriate for maintaining biological activity and providing access for delivery of the product or function, including for example, cylindrical, rectangular, disk-shaped, patch-shaped, ovoid, stellate, or spherical. Moreover, the capsule can be coiled or wrapped into a mesh-like or nested structure. If the capsule is to be retrieved after it is implanted, configurations which tend to lead to migration of the capsules from the site of implantation, such as spherical capsules small enough to travel in the recipient host's blood vessels, are not preferred. Certain shapes, such as rectangles, patches, disks, cylinders, and flat sheets offer greater structural integrity and are preferable where retrieval is desired.

When macrocapsules are used, preferably between 10³ and 10⁸ cells are encapsulated, most preferably 10⁵ to 10⁷ cells are encapsulated in each device. Dosage may be controlled by implanting a fewer or greater number of capsules, preferably between 1 and 10 capsules per patient.

The scaffolding may be coated with extracellular matrix (ECM) molecules. Suitable examples of extracellular matrix molecules include, for example, collagen, laminin, and fibronectin. The surface of the scaffolding may also be modified by treating with plasma irradiation to impart charge to enhance adhesion of cells.

Any suitable method of sealing the capsules may be used, including the use of polymer adhesives or crimping, knotting and heat sealing. In addition, any suitable “dry” sealing method can also be used, as described, e.g., in U.S. Pat. No. 5,653,687, incorporated by reference.

The encapsulated cell devices are implanted according to known techniques. Many implantation sites are contemplated for the devices and methods of this invention. These implantation sites include, but are not limited to, the central nervous system, including the brain, spinal cord (see, U.S. Pat. Nos. 5,106,627, 5,156,844, and 5,554,148, incorporated by reference), and the aqueous and vitreous humors of the eye (see, WO 97/34586, incorporated by reference).

Methods and apparatus for implantation of capsules into the CNS are described in U.S. Pat. No. 5,487,739. Methods and apparatus for implantation of capsules into the eye are described in U.S. Pat. No. 5,904,144, U.S. Pat. No. 6,299,895, U.S. Pat. No. 6,439,427, and US 20030031700.

In one aspect the invention relates to a biocompatible capsule comprising: a core comprising living packaging cells that secrete a viral vector for infection of a target cell, wherein the viral vector is a vector according to the invention; and an external jacket surrounding said core, said jacket comprising a permeable biocompatible material, said material having a porosity selected to permit passage of retroviral vectors of approximately 100 nm diameter thereacross, permitting release of said viral vector from said capsule.

Preferably, the core additionally comprises a matrix, the packaging cells being immobilized by the matrix. According to one embodiment, the jacket comprises a hydrogel or thermoplastic material.

Examples of suitable cells for packaging cell lines include HEK293, NIH3T3, PG13, and ARPE-19 cells. Preferred cells include PG13 and 3T3 cells.

Packaging cell lines may be encapsulated and administered using the methods and compositions disclosed in U.S. Pat. No. 6,027,721 and WO 97/01357 hereby incorporated by reference in their entirety.

### EXAMPLES

### Example 1

Identification of Genes Regulated in the Developing Human Ventral Mesencephalon.

Materials and methods

Samples and RNA Extraction.

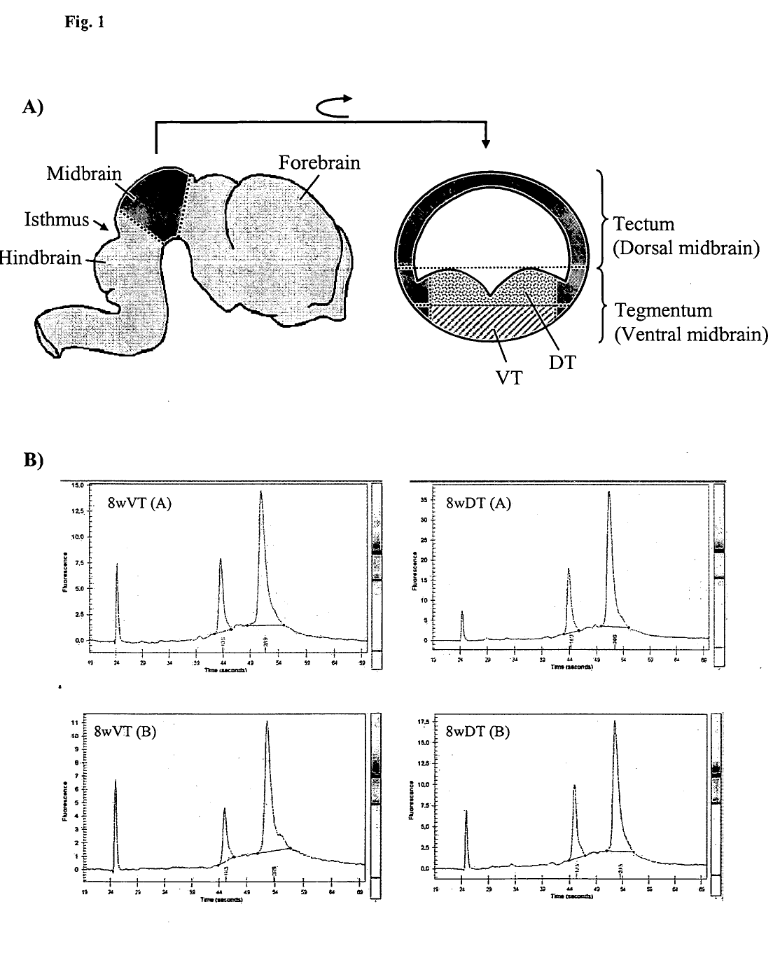

Embryonic tissue was recovered from first trimester routine abortions using standard vacuum aspiration techniques at the Karolinska University Hospital, Huddinge, Sweden. The collection was approved by the Human Ethics Committee of the Huddinge University Hospital, Karolinska Institute, and is in accordance with the guidelines of the Swedish National Board of Health and Welfare (Socialstyrelsen). The GA of each specimen was determined by size and anatomy according to the atlas of England (England, 1988). A total of 14 human embryonic and fetal brain tissue samples, 5 to 10w GA, were used for this study. Cases were rapidly sub dissected into fore- and midbrain. For cases older than 7w GA, midbrain samples were further sub dissected into ventral- and dorsal tegmentum (VT and DT) as illustrated in FIG. 1. After dissection, tissues were instantly frozen in liquid nitrogen and stored at −80° C. Subsequently, total RNA was extracted using Trizol (Invitrogen, Carlsbad, Calif.) according to manufacturers instructions. To concentrate RNA and to remove traces of chromosomal DNA, RNeasy on column DNase digestion with RNase-Free DNase was used (QlAgen, Germany) and RNA subsequently eluted in ultra pure H₂O. The quality of RNA samples for GeneChip analysis was assessed using the Agilent Bioanalyser 2100 and the RNA 600OLabChip. The OD_{260/280} were measured to 1.88+/−0.05 (mean±SD) for the remaining RNA samples to be used for Q-PCR analysis.

Affymetrix Microarray.

Two 8w GA midbrains were sub-dissected into a dorsal and ventral tegmentum and RNA extracted. Two independent rounds of amplification were performed for one sample according to the MessageAMP™ aRNA protocol (Ambion, Austin, Tex.) whereas only one round of amplification was performed for the other, resulting in three ventral samples and three dorsal samples. After purification and fragmentation, 15 μg biotinylated cRNA was hybridized for 16 hours at 45° C. to the Affymetrix Human Genome U133 Chip Set, consisting of two arrays, HG-U133A and HG-U133B (Affymetrix, Santa Clara, Calif.). Samples were first applied to HG-U133A containing mostly well-substantiated human genes and then transferred to HG-U133B, primarily representing ESTs. In total, almost 45,000 probe sets representing more than 39,000 transcripts are present (www.affVmetrix.com). Washing of the arrays and staining with a streptavidin-phycoerytrin conjugate were done using the GeneChip Fluidics Station 400 and subsequent scanning was done with the Affymetrix GeneArray Scanner, all according to manufacturers instructions (Affymetrix, Santa Clara, Calif.).

Array Analysis.

Primary analysis was done using the software-package GenePublisher (Knudsen et at., 2003). In short, qspline (Workman et al., 2002) was used for normalization, bg.adjust (Irizarry et al., 2003) was used for background correction and the Li-Wong method (Li and Wong, 2001) was used for expression index calculation. For further analysis, data obtained from GenePublisher were formatted as a tab-delimited text file and imported into Microsoft Excel2000. For each probe ID the file contained signal values, fold change, a P-value calculated from a t-test and finally gene annotation including accession number and UniGene cluster identifier. Subsequently, a low-stringency filter was applied as follows. First, the average signal values should be larger than 50. Second, the P-value should be smaller than 0.04. Finally, the signal log₂ratio (SLR) should be larger than 0.5, to identify genes that are expressed higher in the VT compared to the DT.

cDNA Synthesis.

Using RT-PCR, all RNA samples used in this study were tested negative for the presence of genomic DNA. The absence of gDNA was verified by RT-PCR reactions using primers against predicted non-transcribed regions on three different chromosomes. These primers were mapped to the Human Genome (assembly May 2004) by In-Silico PCR from UCSC Genome Bioinformatics (http://genome.ucsc.edu/cgi-bin/hgPcr); chr7:93972885-93973150, chr13:90841963-90842175 and chr20:37088292-37088645. 5ng HEK293 gDNA was used as a positive control and dH₂O as a negative control running the following PCR reactions for Chr7 and Chr13: 96° C. 5′; (96° C. 20″, 54° C. 20″, 72° C. 20″)×40, 72° C. 5, 4° C. 0 and 96° C. 5′; (96° C. 20 ″, 65° C. 20″, 72° C. 20″)×40 72° C. 5, 4° C. ∝ for Chr20.

Aliqouts of 2.5μg RNA was used as template for cDNA synthesis with an RNAseH deficient reverse transcriptase derived from MoMLV (SuperScript) and a poly-dT primer. cDNA from all samples were synthesised at the same time using the same mastermix to avoid technical variations.

mCNS Expression Panel

Tissue from different brain regions of E10.5, E11.5, E13.5, P1 and adult mice was isolated and RNA prepared by Trizol extraction as described above. Samples were all DNAse treated, but the absence of gDNA was not verified as the Chr7, Chr13 and Chr20 primer pairs were designed against the human genome. cDNA from all samples were synthesised at the same time as described above using the same mastermix to avoid variations. The expression panel consists of cDNA prepared from the following tissues; dorsal forebrain (DFB), ventral forebrain (VFB), ventral mesencephalon (VM), dorsal mesencephalon (DM) and spinal cord (SC) from 10.5 and 11.5 weeks old embryos. In addition, cortex (CTX), medial and lateral ganglionic eminences (MGE/LGE), DM, VM and SC from 13.5 weeks old embryos were included. Furthermore, from newborn mouse (P1), cerebellum (Cb), CTX, VM, DM, and MGE/LGE were used and finally Cb, CTX, VM, DM, and SC were used from adult mouse. The variation in GAPDH expression across the poanel was Less than 50%. This panel can be used to evaluate expression during CNS development.

Adult Human Expression Panel

After DNAse treatment as described above, cDNA was prepraed from Human master panel (BD, K4008-1), Human Retina (BD, 636579), Human Whole Brain (BD, 64089-1), Human Brain Putamen (BD, 636575), Human Brain Substantia Nigra (BD, 636560), Dorsal root ganglion (BD, 636150) and furthermore from fetal brain (BD, 64094-1) RNA resulting in a panel of 25 tissues. This panel can be used to evaluate expression in adult humans. Quantitative real time PCR.

All primers were designed using Clone Manager Suite 7.1 from Sci Ed Software (Cary, N.C.) and ordered from TAG Copenhagen A/S, Denmark (Table 1). Where possible, primers were designed to be intron spanning amplifying 150 to 400bp. The optimal annealing temperature for each primerset was determined by gradient RT-PCR (PTC-225, MJ Research, Waltham, Mass.) with cDNA prepared from Universal Human Reference RNA (Stratagene, La Jolla, Calif.) or Substantia Nigra total RNA (BD, Palo Alto, Calif.) as template. Gel electrophoresis and melting curve analysis was used to verify that a single PCR product of the predicted size was generated. The product was subsequently isolated using the GENECLEAN II Kit (Qbiogene Inc., Irvine, Calif.) and serially diluted over eight decades to be used for generation of a standard curve. Using approximately 20 ng of each cDNA sample as template, Q-PCR was done in duplicates in an Opticon-2 thermocycler (MJ Research, Waltham, Mass.), using LightCycler-FastStart DNA Master SYBR Green I kit (Roche, Germany). All amplifications were performed in a total volume of 10 μl containing 3 mM MgCl₂, 12% sucrose and 1× reaction buffer included in the LightCycler kit. The PCR cycling profile consisted of a 10 minutes pre-denaturation step at 98° C. followed by 35 three-step cycles; at 98° C. for 10 seconds, at the optimized annealing temperature indicated in Table 3 for the specific gene for 20 seconds and finally at 72° C. for 20 seconds. The relative amount of RNA was calculated according to the standard curve for the particular primer set and normalized to the level of GAPDH, functioning as an internal control message. The variation in GAPDH expression was only 3.2 fold from the lowest to the highest sample over the whole panel of fetal brain cDNA (2.0±0.6, mean ±SD).

To visualize multiple expression profiles, Q-PCR data were formatted as a tab-delimited text file and uploaded to the ClustArray Server v1.01 (http://chianti.ucsd.edu/ClustArray/). Clustering was done with weighted pair-group mathematical average algorithm (WPGMA) using Euclidian distance as distance measure. Purely for cosmetic reasons, some branches of the resulting dendogram were flipped using Adobe Illustrator CS 11.0.0.

Results

Experimental Setup and RNA Quality.

Since only 5-10% of the total cell population in the human fetal VM is made up of DA progenitors/neurons, a gene could be regulated several fold in the DA sub-population, but diluted out in the total RNA mix used for array analysis. Therefore we aimed at dissecting only part of the VT as close as possible to where DA neurons are expected (FIG. 1A). For comparison, the adjacent DT was used. In both cases the neighboring lateral tissue was discarded. This way two closely related regions were compared in which DA neurons are only expected in the VT sample. The sub-dissected 8w tegmental samples yielded RNA amounts between 10-18 μg, which corresponds to the expected 0.5-1 μg RNA per mg brain tissue. Using the Agilent Bioanalyzer 2100, clear 28S and 18S peaks with a [28S/18S] ratio >2 were observed for all samples (FIG. 1B). Also it should be noted that there were no noise between the peaks or low molecular weight contaminations. This indicated overall high quality and non-degraded RNA samples. Aliquots of 5 μg, were amplified resulting in 18-30 μg biotin labeled cRNA, of which 15 μg were used for array analysis on the Human Genome U133 Set.

Few Genes are Differentially Expressed Between VT and DT.

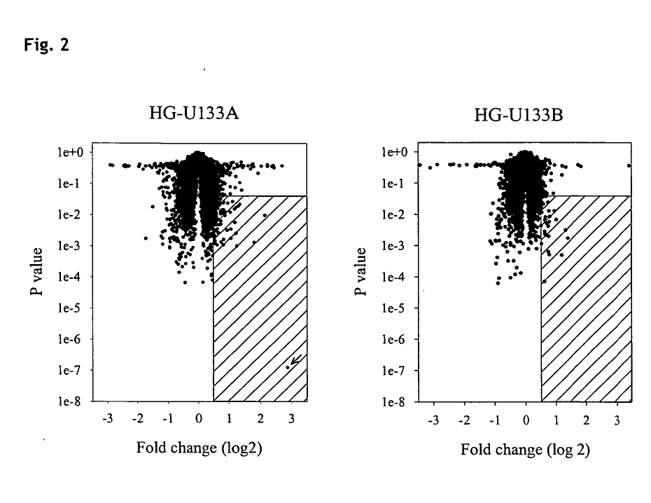

First, we carried out a detailed analysis of data from HG-U133A because in this case the presence of known DA marker genes can be used as a guide to design an effective data filter for detection of similarly regulated genes on the HG-U133B chip. To get an initial overview, data were plotted as a volcano plot (Wolfinger et al., 2001) showing the relationship between P-values and log₂ transformed fold changes (FIG. 2). From the original data, high similarity between the expression profiles of VT and DT is evident from the very narrow plots. Only very few probes are changed more than two fold. However, it was clear that several probes demonstrated higher fold changes for the original data set compared to the permutations (not shown in plot). In particular, one gene stands out with a high fold change of 2.87 (log₂) and a P-value of 1.2×10⁻⁷. This is aldehyde dehydrogenase 1 family member A1 (ALDH1A1), one of the earliest markers of DA phenotype (McCaffery and Drager, 1994). For most differentially expressed probes, P-values higher than 10⁻³ are observed, meaning several potential false positives by chance alone from a statistical point of view. As an alternative, we applied a low stringency filter to the data from HG-U133A in an attempt to filter out differently expressed genes.

Known DA Genes Top the List of Regulated Genes on HG-U133A.

Sorting away probes with signal values smaller than 50, P-values larger than 0.04 or SLR smaller than 0.5, generated a list of 107 probes, which were finally arranged with descending fold changes (Table 2). It can be seen from Table 2, that the most regulated probes represent ALDHlA1, DAT1, VMAT2, TH, Calbindin 1, NURR1 and FOXAL. These are all genes involved in DA differentiation or genes characterizing the more matured phenotype, validating the experimental setup. As mentioned, ALDH1A1 is one of the earliest markers of the DA phenotype (McCaffery and Drager, 1994), whereas the dopamine transporter DAT1 and the vesicular monoamine transporter VMAT2, both represent phenotypic markers for the mature DA neuron. Tyrosine hydroxylase is the initial and rate-limiting enzyme in the synthesis of the catecholamines including dopamine (Goridis and Rohrer, 2002). DA neurons are often defined by their expression of TH in the absence of dopamine [symbol] -hydroxylase, the enzyme that converts dopamine to noradrenaline. Calbindin 1 (28kD) is co-expressed in a subset of TH positive neurons in the developing human VM (Verney et al., 2001) and have also been shown to be expressed in the adult SN (Damier et al., 1999). Therefore Calbindin 1 (28 kD) is often used as a non-phenotypic marker of DA neurons. Three probes for NURR1, an orphan nuclear receptor with essential functions in DA development (Perlmann and Wallen-Mackenzie, 2004), exist on HG-U133A and all are among the most up-regulated as seen in Table 1. FOXA1, also known as HNF3a, is expressed from E8/9 in mice (Ang et al., 1993) and as such it may be one of the earliest cell type specific genes expressed by DA neurons in the VM (Thuret et al., 2004b). FLJ21924 and Cerebellin are also found in the top of the list, but these genes have not previously been associated with DA neurons. PITX2 is indeed expressed in both the developing and adult midbrain, however not by DA neurons (Asbreuk et al., 2002). Instead PITX2 is found in the subthalamic nucleus rostral to the SN and VTA (Martin et al., 2004). As the last probe regulated more than two fold, IGF1 is also detected as differentially expressed in agreement with previous immuno studies (Garcia-Segura et al., 1991).

For probes with less than a two-fold change, several genes known to be expressed by DA neurons are found. All four annotated probes for o-synuclein (SNCA) demonstrated a higher expression in VT compared to DT, supported by immunohistological studies in humans detecting SNCA in SN from 15w GA (Galvin et al., 2001). In parallel, for the D2 dopamine receptor (DRD2), known to be expressed by DA neurons (Meador-Woodruff et al., 1994), all four probes existing on the HG-U133A chip demonstrated a higher expression in VT. The inward rectifying potassium channel KCNJ6, also called Girk2, is detected as higher expressed in the VT in agreement with studies demonstrating high expression in the DA neurons of SN and VTA (Inanobe et al., 1999;Liao et al., 1996). A missense mutation in Girk2 is responsible for the neurological phenotype of the weaver mouse where DA neurons of the SN are selectively degraded (Schmidt et al., 1982).

Although the expression level is low, differential expression is also observed for the receptor tyrosine kinase subunit, RET, in agreement with in situ studies in mouse (Golden et al., 1999). PITX3, a bicoid-related homeodomain transcription factor was likewise identified as higher expressed in VT in agreement with its expression being restricted to the developing eye and most importantly to DA progenitors. In the brain, PITX3 is expressed specifically in the SN and VTA (Smidt et al., 1997) and it has been shown to be required for DA neuron development in SN (Nunes et al., 2003). Although widely distributed in the brain, BDNF expression has been detected in the SNc and VTA and dual in situ hybridization with TH confirms the DA nature of these cells (Seroogy et al., 1994). Finally, EPHA5 has been demonstrated to be expressed by midbrain DA neurons (Yue et al., 1999) which is also the case for ERBB4 (Thuret et al., 2004a; Steiner et al., 1999).

In addition to the welt described DA genes above, several of the identified genes in Table 2 have previously been associated with VM. As an example, SLC17A6, also called VGLUT2, has been detected in SN (Aihara et al., 2000) but also very recently in neonatal rat dopamine neurons in culture (Dal Bo et al., 2004). Furthermore, CHRNA7 (Jones and Wonnacott, 2004), FOXB1 (Alvarez-Bolado et al., 2000), DSCR1L1 (Siddiq et al., 2001), DLK1 (Jensen et al., 2001), TRIM9 (Berti et al., 2002) PCDH11X (Blanco et al., 2000) and TRPM3 (Lee et al., 2003) have been shown to be expressed in the SN/VTA area by PCR or in situ hybridization.

Verification of HG-U133A Data by Literature Mining.

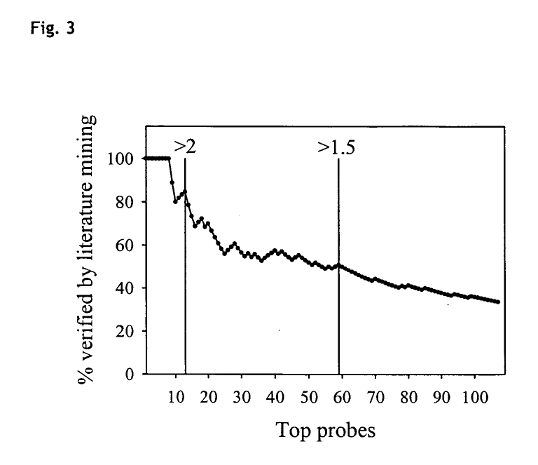

An important part of the validation is looking into the literature as above to compare the results from the array experiment with existing data. Adding up, as can be seen from FIG. 3, a high hit rate, especially in the top of the list, was noted. For probes with a fold change higher than two, an 85% hit rate (11/13) was observed. Including all probes with a fold change higher than 1.5, approximately half of them could be verified (29/59). Towards the bottom of the list, for probes with a fold change less than 1.5, there are only a few known marker genes, indicating that a more stringent filter could have been applied, but that would at the same time limit the opportunity of finding new DA genes. Overall, considering 107 probes, 35 were verified in the literature, corresponding to a hit rate of 33%. Genes that cannot be verified through the existing literature may be the most interesting ones because they are new in a DA setting. Therefore it is important to have additional alternative experimental methods for verification.

Q-PCR Verification on a Large Panel of Developing Human Brain Tissue.

We collected a unique panel of first trimester human fetat tissue, extracted RNA and prepared cDNA for Q-PCR analysis. The panel does not only contain 8w VT and DT, but also includes forebrain and subdissected midbrain tissue from 5-low GA. The 8w samples used for Q-PCR are different biological samples from the ones analyzed by array. This way, array results will be vatidated and at the same time put in a biological context of the developing human brain in the period critical of DA neuron development. However, the panet itself also needs verification to validate dissections. Therefore the expression profile of a number of known DA marker genes, also identified by the array, was analyzed by Q-PCR (FIG. 4A-H). As expected, TH is highly expressed in 5.5-7w midbrain and in the sub dissected VT of 8-10w cases (FIG. 4C). Note however the elevated TH expression in 10wDT (f), indicating that DT and VT were not probably separated for this case. The expression of the other DA marker genes is also increased for 10wDT (f) confirming the presence of DA neurons (FIG. 4A-H). Nevertheless, the corresponding ventral sample, 10wVT(f), still has a much higher expression of the DA marker genes. Also note that the expression of DA marker genes in the 6wMES sample is often tower than the 5.5w and 7wMES, most likely due to inaccuracy in age determination. With this in mind, the overall DA signature is very strong as the marker genes show similar expression profiles in agreement with the literature verifying both the cDNA panel and the array data. Therefore, in conclusion we have a very effective filter to find DA related genes and a strong tool to subsequently verify them by Q-PCR.

Filtering of HG-U133B Data.

We applied the same data filter used for HG-U133A to the HG-U133B chip to find probes up regulated in VT. This resulted in a list of 28 probes mostly annotated to UniGene clusters with no gene associated or to genes of unknown function (Table 3). Several probes are annotated to UniGene clusters next to genes expected to be regulated according to the literature and/or HG-U133A chip data. These would be Hs.27261 (15kb from KCNJ6), Hs.21374 (0.3 kb from SNCA), Hs.181788 (5.6 kb from CBLN1) and Hs.207457 (less than 2.6 kb from CHRNA7). These UniGene clusters may represent not yet recognized untranslated regions from neighboring genes. A few probes can be annotated to known genes. As an example, two probes exist for EPHA5 on the Affymetrix Human Genome U133 Chip Set, one on each chip. The one on HG-U133B demonstrated higher expression in VT which is also the case for the probe on HG-U133A and in agreement with previous studies (Yue et al., 1999).

Differential Expression at 8w GA Verified by Q-PCR.

We chose to verify the fifteen most regulated probes from HG-U133B by Q-PCR, except for EPHA5 and in addition Cerebellin, PITX2, FLJ21924 and DLK1 selected from HG-U133A (FIG. 5. and FIG. 6A-S). In the situation parallel to the array experiment, 8wVT compared to 8wDT, it is evident that differential expression could be verified for 23 out of 25 sequences (p<0.05) (FIG. 5). The two sequences that could not be verified are Cerebellin and FLJ21924, both from HG-U133A. For Cerebellin, 8w (c) and 8w (d) demonstrated a higher expression in DT whereas 8w (e) showed higher expression in the ventral sample (FIG. 6G). For FLJ21924 there was virtually no difference in expression level between VT and DT at 8w (FIG. 6R). Therefore these two genes may represent false positives. In contrast and most strikingly, for all the sequences selected from HG-U133B representing changes down to 56%, differential expression could be confirmed by Q-PCR.

Novel Genes Identified During Ventral Mesencephalic Development.

Looking at the expression of individual transcripts from 5w to low in FIG. 4, it is evident than ALDH1A1, DAT1, TH, PITX3, VMAT2, FOXAL and NURR1 representing known DA marker genes have very similar expression profiles. This group of genes cluster together in the top of the diagram in FIG. 5. For this group of genes the differential expression between VT and DT is very distinct, and a robust signal is observed from the younger midbrain samples coinciding with the time of DA induction in the human fetus (Almqvist et al., 1996; Verney, 1999).

All the genes not previously associated with the DA phenotype are found in the second cluster (II) (FIG. 5). Here the DA signature is still strong for several genes, but in general the overall differential signal is weaker compared to group I (compare FIG. 4 and 6). With a large and very significant up regulation in VT at 8w and increased expression in the young midbrain samples, the DA signature is most evident for SLC10A4 (FIG. 6A). This is a type 2 membrane protein of unknown function belonging to the sodium/bile acid cotransporter family. Q-PCR expression analysis on several adult and a few fetal tissues revealed expression only in the CNS with particular high expression exclusively in SN (FIG. 7A). The Q-PCR analysis of LOC284033 expression also revealed a DA-like profile in the developing human brain (FIG. 6B). Using ProtFun and SignalP (Www.cbs.dtu.dk), the 123 amino acid large hypothetical protein LOC284033, is predicted as a growth factor with a probable signal peptide cleavage site. Q-PCR on several adult and few fetal human tissues revealed expression only in the CNS with particular high expression in adult SN, cerebellum and spinal cord ion addition to the fetal brain (FIG. 7B). However, mouse and rat homologues could not be found and neither did we succeed in heterologous expression of histidine tagged LOC284033. Therefore it was speculated that LOC284033 might represent the UTR region of the neighboring well-conserved FLJ45455 less than 3 kb away. This was supported by Q-PCR analysis with primers in separate exons of FLJ45455 revealing a DA-like expression profile virtually identical to that of LOC284033 (FIG. 6B-D).

LRRC3B, encoding an extremely well conserved leucine-rich repeat-containing protein with a predicted C-terminal transmembrane domain, is differentially expressed between VT and DT in samples older that 8w (FIG. 6E). However, it also seems to be expressed in forebrain and in samples younger than 8w only very limited differential expression is seen. Hs.27261 displaying a DA-like expression profile may represent GIRK2 (FIG. 6F) whereas Cerebellin is a false positive as mentioned earlier (FIG. 6G). Although differential expression within the midbrain could be verified in 8w samples, PITX2 is an exception to the DA profile, as expression in the young forebrain samples is stronger than in the midbrain samples (FIG. 6H). For IGF1, known to be expressed in SN but at the same time widely distributed in the brain (Garcia-Segura et al., 1991), differential expression is seen between VT and DT at 8w GA and older, but no differential expression is observed in the younger samples (FIG. 61).

KIAA 145 identified by two different probes as higher expressed in VT, follow the DA expression profile in great detail when verified with Q-PCR (FIG. 6J-K). As the two different primersets used are detecting the same transcript, it is noteworthy that they also cluster together in FIG. 5. Q-PCR analysis on a panel of adult tissues demonstrated ubiquitous expression but noticeably the highest expression was seen in SN and putamen (FIG. 7C). KIAA1145 encodes a highly conserved transmembrane protein with coiled-coil domains.

For Hs.21374, located very close to o-synuclein, differential expression was seen between VT and DT in samples from 8w, but no differential expression was observed between the younger samples (FIG. 6L). This is also the case for the glucose transporter SLC2A13 (FIG. 6M), the UniGene cluster Hs.9887 (FIG. 6N) and the ORF C20orf100 (FIG. 6O). Hs.9887 is found in several copies on chromosome 15, does not have homologues in other species than the chimpanzee and there is no ORF larger that 100 bases. In contrast, C20orf100 is only found in a single copy on chromosome 20, has both mouse and rat homologues and an ORF with a high mobility group box as found in many transcription factors. Hs.130544, which has the highest fold change on HG-U133B but at the same time very low signals from both VT and DT was detected by Q-PCR in samples only where DA neurons are expected (FIG. 6P). In addition, the three EST's existing for this UniGene is from two 8-9w fetuses and one adult SN respectively. Therefore we sequenced IMAGE:1657840 and IMAGE:1427173 in search of an ORF but found none. Neither did we find homologues in other species or indications of noncoding RNA.

Finally, DLK1 has previously been shown to be expressed in SN and VTA in both rats and humans (Jensen et al., 2001). From FIG. 6S it is seen that even though there is a difference in expression between VT and DT at 8w, DLK1 does not follow the DA signature. In general, DLK1 is higher expressed in the forebrain compared to the corresponding midbrain samples. ELOVL3/CIG30 was not included in FIG. 6 as it was only possible to get a signal from samples older than 8w where DA neurons are expected. Interestingly it has been shown in mouse that ELOVL3/CIG30 and PITX3 genes are arranged in a partially overlapping tail-to-tail array so that the 3 ends of their transcripts are complementary (Tvrdik et al., 1999). This is also the case in rat and human judged from the NCBI Map Viewer (http://www.ncbi.nlm .nih.gov/mapview/).

Expression Analysis in Developing Mouse CNS

Tisue from different brain regions of E10.5, E11.5, E13.5, P1 and adult mice was dissected and RNA isolated. To verify tissue dissections and subsequent RNA isolation and cDNA preparation, the expression profile of several marker genes were investigated (FIG. 8). It is apparent from FIG. 8A, that the expression level of the housekeeping gene GAPDH differs less that 50% between tissues. In contrast, very differentiated expression profiles are observed for ALDH1A1 and OTX2 (FIG. 8B-C). OTX2 is known to be expressed in the forebrain and primarily in the dorsal part of the midbrain with a posterior boundary at the isthmic organiser. As expected, during development, OTX2 is found in the forebrain and (dorsal) midbrain but not in the spinal cord (FIG. 8B). Also as expected, in fetal tissues and at P1, ALDH1A1 is expressed almost exclusively in the ventral midbrain (FIG. 8C) which is also the case for TH (FIG. 8D). The GDNF receptors, GFRA1 and RET are primarily expressed in the ventral midbrain and spinal cord during development (FIG. 8E-F), in agreement with previous studies (Golden et al., 1999). Further, at P1, increased expression of GFRA1 and RET is observed in the striatum. Together, this is evidence of a high quality expression panel of the developing mouse central nervous system (CNS).

SLC10A4 is very similar to the expression profiles of GFRA1 and RET with expression in spinal cord and ventral midbrain during development and later, at P1, increased expression in striatum (FIG. 8G). COBLL1, COBL1 and TRIM9 all have very similar expression profiles (FIG. 8H-J). Generally the expression is low, but there is a marked increase in ventral midbrain and striatum at P1. Interestingly these areas of the brain constitute the nigostriatal pathway, which is a neural pathway connecting the substantia nigra with the striatum. It is one of the major dopamine pathways in the brain, and is particularly involved in the control of movement. Finally, ATXN7L1 and Dscr111 have a more temporal expression profile, but still the highest expression is observed in P1 VM and striatum.

### Example 2

Growth Factors with Midbrain Expression.

Introduction

Factors of importance for DA development do not necessarily have to be differentially expressed between ventral and dorsal tegmentum. In a clinical perspective, secreted factors, especially growth factors, are most interesting as they open up the possibility of protein based treatment strategies. We would like to identify both known and potential growth factors with robust expression in the 8w GA midbrain.

Methods

Human sequence data was downloaded from publicly available databases. Nucleotide sequences were downloaded from Unigene (Unigene ver. 186, ftp://ftp.ncbi.nih.gov/repository/UniGene/Homo_sapiens/Hs.seq.uniq.gz) and Affymetrix (http://www.Affymetrix.com/support/technical/byproduct.affx?product=hgu133) whereas protein sequences were downloaded from International Protein Index (IPI) database (IPI Ver. 3.09, ftp://ftp.ebi.ac.uk/pub/databases/IPI/current/ipi.HUMAN.fasta.gz). Also, Gene Ontology categories were downloaded with mapping information to the IPI-sequences (http://www.ebi.ac.uk/GOA/HUMAN_release.html). All these data were stored in a relational MySQL database. As Affymetrix probe sequences are often located in the non-coding part of an mRNA sequence, mapping to a protein sequence is not trivial. The mapping was performed in two steps. First, the Affymetrix nucleotide probe sequence was aligned to the Unigene data set using the BLASTN program (Altschul et al., 1990). Unigene nucleotide sequences represent full-length mRNA sequences. Next, the identified Unigene sequences were aligned against all human IPI protein sequences by the BLASTX program, which compares nucleotide sequences translated in all six reading frames against peptide sequences. In both alignment steps the criterion for a match was more than 95% sequence identity over the full-length of the shortest sequence.

Results

To identify genes with a robust midbrain expression, we selected probe sets with a signal higher than 150. For the well characterized genes, GO annotation was used directly to identify growth factors (GO:0008083-‘Growth factor activity’) (Table 5).

For probes corresponding to uncharacterized protein sequences, we first used the ProtFun method version 2.2 for function prediction (Jensen et al., 2003). ProtFun is a feature based neural network method which predicts the function of uncharacterized proteins given the amino acid sequence. The program predicts the likelihood and probability for 14 Gene Ontology categories including the “Growth Factor” category. All the sequences predicted as growth factors with odds>2 were next analysed with the SignalP method version 3.0 (Nielsen et al., 1997;Bendtsen et al., 2004). The criteria for a signal peptide prediction was a clevage site length in the range of 15-45 residues and a Dmax score above 0.43. The Dmax score represents a combination of the cleavage site and signal peptide scores. Finally, predicted secreted growth factors belonging to one of the GO categories; unassigned, signal transducer activity (GO:0004871) or binding (GO:0005488), were selected. This way potential secreted growth factors with midbrain expression were identified (Table 6).

Reference List

Aihara Y, Mashima H, Onda H, Hisano S, Kasuya H, Hori T, Yamada S, Tomura H, Yamada Y, Inoue I, Kojima I, Takeda J (2000) Molecular cloning of a novel brain-type Na(+)-dependent inorganic phosphate cotransporter. J Neurochem 74: 2622-2625.

Almqvist PM, Akesson E, Wahlberg LU, Pschera H, Seiger A, Sundstrom E (1996) First trimester development of the human nigrostriatal dopamine system. Exp Neurol 139: 227-237.

Altschul SF, Gish W, Miller W, Myers EW, Lipman DJ (1990) Basic local alignment search tool. J Mol Biol 215: 403-410.

Alvarez-Bolado G, Zhou X, Cecconi F, Gruss P (2000) Expression of Foxbl reveals two strategies for the formation of nuclei in the developing ventral diencephalon. Dev Neurosci 22: 197-206.

Ang S L, Wierda A, Wong D, Stevens K A, Cascio S, Rossant J, Zaret K S (1993) The formation and maintenance of the definitive endoderm lineage in the mouse: involvement of HNF3/forkhead proteins. Development 119: 1301-1315.

Asbreuk C H, Vogelaar C F, Hellemons A, Smidt M P, Burbach J P (2002) CNS expression pattern of Lmx1b and coexpression with ptx genes suggest functional cooperativity in the development of forebrain motor control systems. Mof Cell Neurosci 21: 410-420.

Ashburner M, Ball C A, Blake J A, Botstein D, Butler H, Cherry J M, Davis A P, Dolinski K, Dwight S S, Eppig J T, Harris M A, Hill D P, Issel-Tarver L, Kasarskis A, Lewis S, Matese J C, Richardson J E, Ringwald M, Rubin G M, Sherlock G (2000) Gene ontology: tool for the unification of biology. The Gene Ontology Consortium. Nat Genet 25: 25-29.

Bendtsen J D, Nielsen H, von Heijne G, Brunak S (2004) Improved prediction of signal peptides: SignalP 3.0. J Mol Biol 340: 783-795.

Berti C, Messali S, Ballabio A, Reymond A, Meroni G (2002) TRIM9 is specifically expressed in the embryonic and adult nervous system. Mech Dev 113: 159-162.

Bjorklund L M, Sanchez-Pernaute R, Chung S, Andersson T, Chen I Y, McNaught K S, Brownell A L, Jenkins B G, Wahlestedt C, Kim K S, Isacson O (2002) Embryonic stem cells develop into functional dopaminergic neurons after transplantation in a Parkinson rat model. Proc Natl Acad Sci U S A 99: 2344-2349.

Blanco P, Sargent C A, Boucher C A, Mitchell M, Affara N A (2000) Conservation of PCDHX in mammals; expression of human X/Y genes predominantly in brain. Mamm Genome 11: 906-914.

Burbach J P, Smits S, Smidt M P (2003) Transcription factors in the development of midbrain dopamine neurons. Ann N Y Acad Sci 991: 61-68.

Dal Bo G, St Gelais F, Danik M, Williams S, Cotton M, Trudeau L E (2004) Dopamine neurons in culture express VGLUT2 explaining their capacity to release glutamate at synapses in addition to dopamine. J Neurochem 88: 1398-1405.

Damier P, Hirsch E C, Agid Y, Graybiel A M (1999) The substantia nigra of the human brain. I. Nigrosomes and the nigral matrix, a compartmental organization based on calbindin D(28K) immunohistochemistry. Brain 122 ( Pt 8): 1421-1436.

England, M. A. A Color Atlas of Life before Birth. Normal Fetal Development. Wolfe Medical Publication, London. 1988.

Freed C R, Greene P E, Breeze R E, Tsai W Y, DuMouchel W, Kao R, Dillon S, Winfield H, Culver S, Trojanowski J Q, Eidelberg D, Fahn S (2001) Transplantation of embryonic dopamine neurons for severe Parkinson's disease. N EngI J Med 344: 710-719.

Galli R, Binda E, Orfanelli U, Cipelletti B, Gritti A, De Vitis S, Fiocco R, Foroni C, Dimeco F, Vescovi A (2004) Isolation and characterization of tumorigenic, stem-like neural precursors from human glioblastoma. Cancer Res 64: 7011-7021.

Galvin J E, Schuck T M, Lee V M, Trojanowski J Q (2001) Differential expression and distribution of alpha-, beta-, and gamma-synuclein in the developing human substantia nigra. Exp Neurol 168: 347-355.

Garcia-Segura L M, Perez J, Pons S, Rejas M T, Torres-Aleman I (1991) Localization of insulin-like growth factor I (IGF-I)-like immunoreactivity in the developing and adult rat brain. Brain Res 560: 167-174.

Golden J P, DeMaro J A, Osborne P A, Milbrandt J, Johnson E M, Jr. (1999) Expression of neurturin, GDNF, and GDNF family-receptor mRNA in the developing and mature mouse. Exp Neurol 158: 504-528.

Goridis C, Rohrer H (2002) Specification of catecholaminergic and serotonergic neurons. Nat Rev Neurosci 3: 531-541.

Greene J G, Dingledine R, Greenamyre J T (2005) Gene expression profiling of rat midbrain dopamine neurons: implications for selective vulnerability in parkinsonism. Neurobiol Dis 18: 19-31.

Grimm J, Mueller A, Hefti F, Rosenthal A (2004) Molecular basis for catecholaminergic neuron diversity. Proc Natl Acad Sci U S A 101: 13891-13896.

Hynes M, Rosenthal A (1999) Specification of dopaminergic and serotonergic neurons in the vertebrate CNS. Curr Opin Neurobiol 9: 26-36.

Inanobe A, Yoshimoto Y, Horio Y, Morishige K I, Hibino H, Matsumoto S, Tokunaga Y, Maeda T, Hata Y, Takai Y, Kurachi Y (1999) Characterization of G-protein-gated K+ channels composed of Kir3.2 subunits in dopaminergic neurons of the substantia nigra. J Neurosci 19: 1006-1017.

Irizarry R A, Hobbs B, Collin F, Beazer-Barclay Y D, Antonellis K J, Scherf U, Speed T P (2003) Exploration, normalization, and summaries of high density oligonucleotide array probe level data. Biostatistics 4: 249-264.

Jensen C H, Meyer M, Schroder H D, Kliem A, Zimmer J, Teisner B (2001) Neurons in the monoaminergic nuclei of the rat and human central nervous system express FA1/dlk. Neuroreport 12: 3959-3963.

Jensen U, Gupta R, Staerfeldt H H, Brunak S (2003) Prediction of human protein function according to Gene Ontology categories. Bioinformatics 19: 635-642.

Jones I W, Wonnacott S (2004) Precise localization of alpha7 nicotinic acetylcholine receptors on glutamatergic axon terminals in the rat ventral tegmental area. J Neurosci 24: 11244-11252.

Kim J H, Auerbach J M, Rodriguez-Gomez J A, Velasco I, Gavin D, Lumelsky N, Lee S H, Nguyen J, Sanchez-Pernaute R, Bankiewicz K, McKay R (2002) Dopamine neurons derived from embryonic stem cells function in an animal model of Parkinsons disease. Nature 418: 50-56.

Kirik D, Georgievska B, Bjorklund A (2004) Localized striatal delivery of GDNF as a treatment for Parkinson disease. Nat Neurosci 7: 105-110.

Knudsen S, Workman C, Sicheritz-Ponten T, Friis C (2003) GenePublisher: Automated analysis of DNA microarray data. Nucleic Acids Res 31: 3471-3476.

Lang A E, Lozano A M (1998a) Parkinsons disease. First of two parts. N Engl J Med 339: 1044-1053.

Lang A E, Lozano A M (1998b) Parkinsons disease. Second of two parts. N EngI J Med 339: 1130-1143.

Lee N, Chen J, Sun L, Wu S, Gray K R, Rich A, Huang M, Lin J H, Feder J N, Janovitz E B, Levesque P C, Blanar M A (2003) Expression and characterization of human transient receptor potential melastatin 3 (hTRPM3). J Biol Chem 278: 20890-20897.

Li C, Wong W H (2001) Model-based analysis of oligonucleotide arrays: model validation, design issues and standard error application. Genome Biol 2: RESEARCH0032.

Liao Y J, Jan Y N, Jan L Y (1996) Heteromultimerization of G-protein-gated inwardly rectifying K+ channel proteins GIRK1 and GIRK2 and their altered expression in weaver brain. J Neurosci 16: 7137-7150.

Lin L F, Doherty D H, Lile J D, Bektesh S, Collins F (1993) GDNF: a glial cell line-derived neurotrophic factor for midbrain dopaminergic neurons. Science 260: 1130-1132.

Martin D M, Skidmore J M, Philips S T, Vieira C, Gage P J, Condie B G, Raphael Y, Martinez S, Camper S A (2004) PITX2 is required for normal development of neurons in the mouse subthalamic nucleus and midbrain. Dev Biol 267: 93-108.

McCaffery P, Drager U C (1994) High levels of a retinoic acid-generating dehydrogenase in the meso-telencephalic dopamine system. Proc Natl Acad Sci U S A 91: 7772-7776.

Meador-Woodruff J H, Damask S P, Watson S J, Jr. (1994) Differential expression of autoreceptors in the ascending dopamine systems of the human brain. Proc Natl Acad Sci U S A 91: 8297-8301.

Nielsen H, Engelbrecht J, Brunak S, von Heijne G (1997) Identification of prokaryotic and eukaryotic signal peptides and prediction of their cleavage sites. Protein Eng 10: 1-6.

Nunes I, Tovmasian L T, Silva R M, Burke R E, Goff S P (2003) Pitx3 is required for development of substantia nigra dopaminergic neurons. Proc Natl Acad Sci U S A 100: 4245-4250.

Olanow C W, Goetz C G, Kordower J H, Stoessl A J, Sossi V, Brin M F, Shannon K M, Nauert G M, Perl D P, Godbold J, Freeman T B (2003) A double-blind controlled trial of bilateral fetal nigral transplantation in Parkinson's disease. Ann Neurol 54:-403-414.

Perlmann T, Wallen-Mackenzie A (2004) Nurr1, an orphan nuclear receptor with essential functions in developing dopamine cells. Cell Tissue Res 318: 45-52.

Polymeropoulos M H, Higgins J J, Golbe L I, Johnson W G, Ide S E, Di Iorio G, Sanges G, Stenroos E S, Pho L T, Schaffer A A, Lazzarini A M, Nussbaum R L, Duvoisin R C (1996) Mapping of a gene for Parkinson's disease to chromosome 4q21 -q23. Science 274: 1197-1199.

Roybon L, Christophersen N S, Brundin P, Li J Y (2004) Stem cell therapy for Parkinson's disease: where do we stand? Cell Tissue Res 318: 261-273.

Schmidt M J, Sawyer B D, Perry K W, Fuller R W, Foreman M M, Ghetti B (1982) Dopamine deficiency in the weaver mutant mouse. J Neurosci 2: 376-380.

Seroogy K B, Lundgren K H, Tran T M, Guthrie K M, Isackson P J, Gall C M (1994) Dopaminergic neurons in rat ventral midbrain express brain-derived neurotrophic factor and neurotrophin-3 mRNAs. J Comp Neurol 342: 321-334.

Siddiq A, Miyazaki T, Takagishi Y, Kanou Y, Hayasaka S, Inouye M, Seo H, Murata Y (2001) Expression of ZAKI-4 messenger ribonucleic acid in the brain during rat development and the effect of hypothyroidism. Endocrinology 142: 1752-1759.

Simon H H, Saueressig H, Wurst W, Goulding M D, O'Leary D D (2001) Fate of midbrain dopaminergic neurons controlled by the engrailed genes. J Neurosci 21: 3126-3134.

Simon H H, Thuret S, Alberi L (2004) Midbrain dopaminergic neurons: control of their cell fate by the engrailed transcription factors. Cell Tissue Res 318: 53-61.

Smidt M P, Asbreuk C H, Cox J J, Chen H, Johnson R L, Burbach J P (2000) A second independent pathway for development of mesencephalic dopaminergic neurons requires Lmx1b. Nat Neurosci 3: 337-341.

Smidt M P, van Schaick H S, Lanctot C, Tremblay J J, Cox J J, van der Kleij A A, Wolterink G, Drouin J, Burbach J P (1997) A homeodomain gene Ptx3 has highly restricted brain expression in mesencephalic dopaminergic neurons. Proc Natl Acad Sci U S A 94: 13305-13310.

Steiner H, Blum M, Kitai S T, Fedi P (1999) Differential expression of ErbB3 and ErbB4 neuregulin receptors in dopamine neurons and forebrain areas of the adult rat. Exp Neurol 159: 494-503.

Studer L, Tabar V, McKay R D (1998) Transplantation of expanded mesencephalic precursors leads to recovery in parkinsonian rats. Nat Neurosci 1: 290-295.

Takagi Y, Takahashi J, Saiki H, Morizane A, Hayashi T, Kishi Y, Fukuda H, Okamoto Y, Koyanagi M, Ideguchi M, Hayashi H, Imazato T, Kawasaki H, Suemori H, Omachi S, Iida H, Itoh N, Nakatsuji N, Sasai Y, Hashimoto N (2005) Dopaminergic neurons generated from monkey embryonic stem cells function in a Parkinson primate model. J Clin Invest 115: 102-109.

Thuret S, Alavian K N, Gassmann M, Lloyd C K, Smits S M, Smidt M P, Klein R, Dyck H, Simon HH (2004a) The neuregulin receptor, ErbB4, is not required for normal development and adult maintenance of the substantia nigra pars compacta. J Neurochem 91: 1302-1311.

Thuret S, Bhatt L, O'Leary D D, Simon H H (2004b) Identification and developmental analysis of genes expressed by dopaminergic neurons of the substantia nigra pars compacta. Mol Cell Neurosci 25: 394-405.

Tvrdik P, Asadi A, Kozak L P, Nuglozeh E, Parente F, Nedergaard J, Jacobsson A (1999) Cig30and Pitx3 genes are arranged in a partially overlapping tail-to-tail array resulting in complementary transcripts. J Biol Chem 274: 26387-26392.

Verney C (1999) Distribution of the catecholaminergic neurons in the central nervous system of human embryos and fetuses. Microsc Res Tech 46: 24-47.

Verney C, Zecevic N, Ezan P (2001) Expression of calbindin D28K in the dopaminergic mesotelencephalic system in embryonic and fetal human brain. J Comp Neurol 429: 45-58.

Vitalis T, Cases O, Parnavelas J G (2005) Development of the dopaminergic neurons in the rodent brainstem. Exp Neurol 191 Suppl 1: S104-S112.

Winkler C, Kirik D, Bjorklund A (2005) Cell transplantation in Parkinson's disease: how can we make it work? Trends Neurosci 28: 86-92.

Wolfinger R D, Gibson G, Wolfinger E D, Bennett L, Hamadeh H, Bushel P, Afshari C, Paules R S (2001) Assessing gene significance from cDNA microarray expression data via mixed models. J Comput Biol 8: 625-637.

Workman C, Jensen U, Jarmer H, Berka R, Gautier L, Nielser H B, Saxild H H, Nielsen C, Brunak S, Knudsen S (2002) A new non-linear normalization method for reducing variability in DNA microarray experiments. Genome Biol 3: research0048.

Yao F, Yu F, Gong L, Taube D, Rao D D, MacKenzie R G (2005) Microarray analysis of fluoro-gold labeled rat dopamine neurons harvested by laser capture microdissection. J Neurosci Methods 143: 95-106.

Yue Y, Widmer D A, Halladay A K, Cerretti D P, Wagner G C, Dreyer J L, Zhou R (1999) Specification of distinct dopaminergic neural pathways: roles of the Eph family receptor EphB1 and ligand ephrin-B2. J Neurosci 19: 2090-2101.

Zetterstrom R H, Solomin L, Jansson L, Hoffer B J, Olson L, Perlmann T (1997) Dopamine neuron agenesis in Nurr1-deficient mice. Science 276: 248-250.

Tables:

Table 1. List of primers used in this study. Q-PCR was done as described in the text.

Table 2. Genes up-regulated in the ventral tegmentum. Data from HG-U 133A was sorted as described in the text and listed with descending fold changes. Some genes are represented by several probes. Gene, in the first column, refers to the approved gene symbol defmed by the HUGO Gene Nomenclature Committee, whereas the second column refers to what may be a more commonly known alias. GeneID can be used directly for looking up the specific sequence in Entrez Gene at National Center for Biotechnology information (NCBI)

(http://www.ncbi.nlm.nih.gov/entrez/query.fcgi?db=gene). The sequences available from NCBI are incorporated by reference. Where possible, for each gene, the GO annotation column refers to the molecular finction defined by the Gene Ontology Consortium (Ashburner et al., 2000). GO categories were annotated using AmiGO (http://wwwgodatabase.or/cgi-bin/amigo/go.cgi) and checked manually. VT and DT refer to the average probe signal from ventral and dorsal tegmental samples respectively. Finally, in the last column, the change in gene expression expressed as VT/DT. Genes in bold are verified by literature as differentially expressed (see text for references), whereas genes analyzed by Q-PCR in FIG. 4 and FIG. 6 are marked by an asterisk (*).

Table 3. Sequences from HG-U133B up regulated in the ventral tegmentumr Data from HG-U133B was sorted as described for HG-U133A. The UniGene entry in the first column can be used for looking up sequences at http:www.ncbi.nlm.nih.gov/entrez/query.fcgi?db=unigene. The sequences available from UniGene are incorporated by reference. For some of the sequences a known or hypothetical gene is associated which is indicated in the second column by the official gene names according to HUGO. The remaining UniGene sequences are annotated as UNKNOWN. VT and DT refer to the average probe signal from ventral and dorsal tegmentum respectively whereas the last column shows the change in expression as VT/DT. Sequences analyzed by Q-PCR in FIG. 6 are marked by an asterisk (*).

Table 4. Reference to sequences. References to public databases for the sequences encoding transmembrane proteins or transcription factors identified in Table 2 and 3. Gene, in the first column, refers to the approved gene symbol defmed by the HUGO Gene Nomenclature Committee and the second column refers to the GeneID, which can be used directly for looking up the specific sequence in Entrez Gene at National Center for Biotechnology information (NCBI)

(http://www.ncbi.nlm.nih.gov/entrez/query.fcgi?db=gene). Furthermore the UniGene ID can be used for looking up sequences in a non-redundant set of gene-oriented clusters at

http:/www.ncbi.nlm.nih.gov/entrez/guery.fcgi?db=unigene. Finally, RefSeq Transcript ID refers to a stable non-redundant set of sequences for gene identification, which can be found at

http://www.ncbi.nlm.nih.gov/RefSeq/ and many other NCBI databases. The sequences available from NCBI, UniGene and RefSeq are incorporated by reference.

Table 5. Growth factors expressed in 8w GA midbrain. Probes with an average signal higher than 150 belonging to the GO category “growth factor activity” (GO:0008083). The genes are sorted according to the average probe signal. Gene, in the first column, refers to the approved gene symbol defmed by the HUGO Gene Nomenclature Committee and the second column refers to the affymetrix probe ID. Average, in the third column, is the average signal from VT and DT for the particular probe. UniGene ID can be used for looking up sequences in a non-redundant set of gene-oriented clusters at http://www.ncbi.nlm.nih.gov/entrez/query.fcgi?db=unigene. IPI ID can be used for looking up protein sequences at http://www.ebi.ac.uk/IPI/IPIhelp.html. RefSeq ID refers to a stable non-redundant set of sequences for gene identification, which can be found at http://www.ncbi.nlm.nih.gov/RefSeq/ and many other NCBI databases. The sequences available from UniGene, IPI and RefSeq are incorporated by reference.

Table 6. Predicted growth factors expressed in 8w GA midbrain. Some genes are represented by more than one probe set. For details see legend to Table 5. The ProtFun score is shown in the fourth column.

TABLE 1
List of primers used in this study.

| | | Annealing | Product |
|---|---|---|---|
| Target | Forward / Reverse | (° C.) | (bp) |
| Chr7 | ATAAAGGTTCCTGTGATGTCAG | 54 | 266 |
| | GTAAGTTATTTAGATGATTTTG | | |
| | AATGC | | |
| Chr13 | ATTAAACATGATAAAAGGGATT | 54 | 213 |
| | CTTACATAATGAGCACTTAGAG | | |
| | CAGGAT | | |
| Chr20 | AAACGTGTGCTCTTTTCCCCAT | 65 | 354 |
| | CTTATGCTAGCCCTCTCTGGAT | | |
| | TCCTTCTTC | | |
| ALDH1A1 | GGGCAGCCATTTCTTCTCAC | 65 | 299 |
| | CTTCTTAGCCCGCTCAACAC | | |
| DAT1 | AGTGTGCCCATGAGTAAGAG | 65 | 340 |
| | TCCCAGCAATGACCATGAAG | | |
| VMAT2 | GGACAACATGCTGCTCACTG | 65 | 178 |
| | ATTCCCGGTGACCATAGTCG | | |
| TH | GAGGGGAAGGCCGTGCTAAA | 65 | 174 |
| | GAGGCGCACGAAGTACTCCA | | |
| NURR1 | AGACCTTCGCTGTGCCCAAC | 65 | 366 |
| | TCCCAACAGCCAGGCACTTC | | |
| FOXA1 | GGGCTGGATGGTTGTATTGG | 66 | 212 |
| | ATGTTGCCGCTCGTAGTC | | |
| FLJ21924 | TGGCGGTAACAGTCCATCAG | 65 | 204 |
| | GCAGTTGGGCTAGTAGTTGC | | |
| CBLN1 | AATGAGACGGAGCCCATC | 64 | 247 |
| | TGAAAGTGCTGCGTTCTG | | |
| PITX2 | ACCTTACGGAAGCCCGAGTC | 55 | 204 |
| | TGGATAGGGAGGCGGATGTA | | |
| IGF1 | TGGTGGATGCTCTTCAGTTC | 65 | 261 |
| | TCCCTCTACTTGCGTTCTTC | | |
| PITX3 | GCCGCTACTGGGAGTCTG | 66 | 164 |
| | CTGTGCTCCTGGCCCTTG | | |
| DLK1 | TTCACGGACTCTGTGGAGAAC | 65 | 312 |
| | CGCAGAAATTGCCTGAGAAGC | | |
| GFRa1 | CAAGCACAGCTACGGAATGC | 60 | 309 |
| | TGAGGCTACTGGAGTCTATG | | |
| Hs.130544 | CTCCAACATGTCGAGTCTAC | 66 | 193 |
| | TTGTGATGGAGCCATACC | | |
| Hs.370410 | GTGAGGCTTGTTGCAGAA | 60 | 129 |
| | TAATGGCACCTGCTTTGG | | |
| Hs.9887 | TGTAGATGGGAACATGGTTTG | 60 | 229 |
| | GCAGTCTAAGTGAAACCCATA | | |
| Hs.27261 | GTATGTCTGTGCACGGACAATG | 60 | 164 |
| | GTTTCCCTAACACAGCGAGTTC | | |
| Hs.132591 | GGTGGCTGACTACATTGTG | 58 | 200 |
| | TGGGTGGAAGATGGAAGAG | | |
| Hs.30875 | GTCACCAGCCTCCAACTTC | 60 | 165 |
| | CCCAGATTCGGACAGGATG | | |
| Hs.524341 | CTACACGTCCAAACTGTCTC | 66 | 326 |
| | CAGCATAGGCTAGGATGACT | | |
| Hs.21374 | CCCACTTGGAATACCAAACC | 60 | 197 |
| | TTCACTCCCGAGACATTCAC | | |
| B4GALT6 | CCTTTTGCCAGCCAAGATAC | 66 | 213 |
| | AGTTCTCTCAACCGGCATAC | | |
| Hs.370410 | ACCATTCACCGCCTTCAGCC | 66 | 342 |
| | TGCCTAAGTCCCGCTCACAC | | |
| Hs.517868 | CGGGCTTCTTCAGAAGTACC | 61 | 245 |
| | AGGAGGAGACACATGGAGAG | | |
| Hs.302130 | AAGGAACGCAAGGGCTTCAACA | 65 | 193 |
| | GACCCAGGACCAGAATTTGAC | | |
| Hs.7413 | GCCCAACAGATTCCTGTGATG | 61 | 213 |
| | CGCACTGTCTTCTCAGTCAAC | | |
| Hs.26608 | TGCCAAACCGTTTCGTCATC | 66 | 217 |
| | TGCAAACGACGTCAAGAACC | | |

TABLE 2
Genes up-regulated in the ventral tegmentum.

| Gene | Alias | Gene ID | GO annotation | VT | DT | Change |
|---|---|---|---|---|---|---|
| ALDH1A1* | AHD2 | 216 | Catalytic | 316 | 43 | 7.31 |
| SLC6A3* | DAT1 | 6531 | Transporter | 462 | 104 | 4.44 |
| SLC18A2* | VMAT2 | 6571 | Transporter | 214 | 61 | 3.51 |
| TH* | | 7054 | Catalytic | 655 | 257 | 2.55 |
| CALB1 | Calbindin 1, 28 kD | 793 | Binding | 337 | 137 | 2.46 |
| NR4A2* | Nurr1 | 4929 | Transcription | 908 | 381 | 2.38 |
| FOXA1* | HNF3A | 3169 | Transcription | 648 | 278 | 2.33 |
| NR4A2* | Nurr1 | 4929 | Transcription | 507 | 217 | 2.33 |
| FLJ21924* | | 79832 | — | 374 | 164 | 2.28 |
| CBLN1* | | 869 | — | 185 | 82 | 2.27 |
| PITX2* | PTX2 | 5308 | Transcription | 266 | 122 | 2.17 |
| NR4A2* | Nurr1 | 4929 | Transcription | 724 | 344 | 2.10 |
| IGF1* | somatomedin C | 3479 | Signal transduction | 112 | 53 | 2.10 |
| STK17A | DRAK1 | 9263 | Catalytic | 68 | 35 | 1.96 |
| CADPS2 | | 93664 | — | 234 | 121 | 1.93 |
| PAQR6 | FLJ22672 | 79957 | Signal transduction | 705 | 383 | 1.84 |
| GABRB1 | | 2560 | Transporter | 178 | 97 | 1.84 |
| SNCA | α-synuclein | 6622 | — | 352 | 199 | 1.78 |
| PPP1R3C | PP1R5 | 5507 | Catalytic | 327 | 184 | 1.78 |
| SNCA | α-synuclein | 6622 | — | 555 | 315 | 1.77 |
| SCARB2 | CD36L2 | 950 | Signal transduction | 490 | 278 | 1.77 |
| COBLL1 | KIAA0977 | 22837 | — | 154 | 89 | 1.73 |
| ARHGAP6 | RHOGAP6 | 395 | Signal transduction | 111 | 64 | 1.72 |
| MYRIP | exophilin | 25924 | Binding | 235 | 138 | 1.71 |
| HBLD2 | hIscA | 81689 | — | 150 | 89 | 1.69 |
| DRD2 | D2R | 1813 | Signal transduction | 402 | 239 | 1.68 |
| KCNJ6 | GIRK2 | 3763 | Transporter | 473 | 283 | 1.67 |
| RET | HSCH1 | 5979 | Signal transduction | 64 | 38 | 1.67 |
| FLJ11627 | | — | — | 96 | 57 | 1.67 |
| COPG2 | 2-COP | 26958 | Transporter | 63 | 38 | 1.66 |
| FLJ20130 | | 54830 | Transporter | 121 | 73 | 1.66 |
| FOXB1 | FKH5 | 27023 | Transcription | 170 | 102 | 1.66 |
| RRAS2 | TC21 | 22800 | Binding | 245 | 149 | 1.64 |
| PITX3* | PTX3 | 5309 | Transcription | 167 | 102 | 1.64 |
| PHLDA1 | TDAG51 | 22822 | — | 173 | 106 | 1.62 |
| CORIN | CRN | 10699 | Catalytic | 90 | 56 | 1.62 |
| BDNF | | 627 | Signal transduction | 128 | 80 | 1.61 |
| SNCA | α-synuclein | 6622 | — | 326 | 203 | 1.60 |
| DSCR1L1 | ZAKI-4 | 10231 | Binding | 531 | 331 | 1.60 |
| DLK1* | FA-1 | 8788 | Binding | 289 | 181 | 1.59 |
| ATXN7L1 | KIAA1218 | 57485 | — | 130 | 82 | 1.58 |
| TRIM9 | SPRING | 114088 | Catalytic | 643 | 408 | 1.58 |
| RBMS1 | YC1 | 5937 | Binding | 236 | 150 | 1.57 |
| ZNF73 | | 7624 | Transcription | 102 | 65 | 1.57 |
| GRM8 | GLUR8 | 2918 | Signal transduction | 73 | 47 | 1.56 |
| SLC17A6 | VGLUT2 | 57084 | Transporter | 619 | 398 | 1.56 |
| PCDH11X | | 83259 | Binding | 235 | 151 | 1.56 |
| BCL2L2 | Bcl-w | 599 | — | 289 | 187 | 1.55 |
| C10orf3 | FLJ10540 | 55165 | — | 85 | 55 | 1.55 |
| NEBL | Nebulette | 10529 | Binding | 139 | 91 | 1.54 |
| PPEF2 | | 5470 | Catalytic | 85 | 55 | 1.54 |
| SNCA | α-synuclein | 6622 | — | 310 | 203 | 1.53 |
| CLDN11 | Claudin 11 | 5010 | Structural | 106 | 70 | 1.52 |
| UBE2D1 | UBC4/5 | 7321 | Catalytic | 438 | 288 | 1.52 |
| PLS3 | Plastin 3 | 5358 | Bining | 104 | 69 | 1.52 |
| EPHA5 | EHK1 | 2044 | Signal transduction | 116 | 77 | 1.51 |
| IL-17RC | MGC10763 | 84818 | Signal transduction | 186 | 124 | 1.51 |
| CHRNA7 | NACHRA7 | 1139 | Signal transduction | 224 | 149 | 1.51 |
| ERBB4 | HER4 | 2066 | Signal transduction | 670 | 444 | 1.51 |
| ADRA1B | ADRA1 | 147 | Signal transduction | 261 | 175 | 1.49 |
| HPS1 | MGC5277 | 3257 | — | 92 | 61 | 1.49 |
| RAB3B | | 5865 | Catalytic | 121 | 81 | 1.49 |
| SDC1 | SYND1 | 6382 | Binding | 143 | 96 | 1.49 |
| OR12D2 | | 26529 | Signal transduction | 174 | 117 | 1.48 |
| RRP22 | | 10633 | Catalytic | 130 | 87 | 1.48 |
| RAB5A | RAB5 | 5868 | Catalytic | 127 | 86 | 1.47 |
| CYP1B1 | CP1B | 1545 | Transporter | 61 | 42 | 1.47 |
| YES1 | C-YES | 7525 | Catalytic | 81 | 55 | 1.47 |
| RGS5 | MSTP032 | 8490 | Enzyme regulator | 109 | 74 | 1.47 |
| DRD2 | D2R | 1813 | Signal transduction | 369 | 251 | 1.46 |
| AKAP12 | AKAP250 | 9590 | Binding | 533 | 365 | 1.46 |
| CNNM2 | ACDP2 | 54805 | — | 176 | 121 | 1.46 |
| B3GALT2 | GLCT2 | 8707 | Catalytic | 171 | 117 | 1.46 |
| GRB10 | MEG1 | 2887 | Signal transduction | 659 | 448 | 1.46 |
| FPRL1 | LXA4R | 2358 | Signal transduction | 62 | 42 | 1.46 |
| C1ORF1 | Po42 | 711 | — | 684 | 467 | 1.46 |
| OAS2 | P69 | 4939 | Catalytic | 89 | 61 | 1.46 |
| DRD2 | D2R | 1813 | Signal transduction | 235 | 160 | 1.46 |
| RAB3B | | 5865 | Signal transduction | 154 | 106 | 1.45 |
| DRD2 | D2R | 1813 | Signal transduction | 118 | 81 | 1.45 |
| DCN | DSPG2 | 1634 | — | 193 | 133 | 1.45 |
| PLCL3 | KIAA1069 | 23007 | Signal transduction | 227 | 156 | 1.45 |
| ZW10 | HZW10 | 9183 | Binding | 141 | 97 | 1.45 |
| ADCY2 | HBAC2 | 108 | Catalytic | 449 | 309 | 1.45 |
| SNCA | α-synuclein | 6622 | — | 77 | 53 | 1.45 |
| DNAJC6 | DJC6 | 9829 | Catalytic | 643 | 446 | 1.44 |
| LOH11CR2A | BCSC-1 | 4013 | — | 287 | 199 | 1.44 |
| RNF17 | Mmip-2 | 56163 | Catalytic | 71 | 49 | 1.44 |
| ANK3 | Ankyrin-G | 288 | Binding | 538 | 373 | 1.44 |
| TA-LRRP | KIAA0231 | 23507 | — | 92 | 63 | 1.44 |
| SPPH1 | SPPase1 | 81537 | Catalytic | 120 | 83 | 1.44 |
| CD14 | | 929 | Signal transduction | 79 | 55 | 1.44 |
| MGC3771 | | 81854 | — | 219 | 152 | 1.43 |
| IGF1* | somatomedin C | 3479 | Signal transduction | 118 | 83 | 1.43 |
| NUDT4 | DIPP2 | 11163 | Catalytic | 59 | 41 | 1.43 |
| SV2B | KIAA0735 | 9899 | Transporter | 261 | 182 | 1.43 |
| SH3GL3 | EEN-B2 | 6457 | Binding | 633 | 441 | 1.43 |
| LRRTM4 | FLJ12568 | 80059 | — | 155 | 108 | 1.43 |
| TRPM3 | MLSN2 | 80036 | Signal transduction | 372 | 260 | 1.43 |
| SPAG4 | ACR55 | 6676 | — | 72 | 51 | 1.42 |
| DOCK9 | ZIZ1 | 23348 | Binding | 167 | 117 | 1.42 |
| WDR1 | AIP1 | 9948 | Binding | 92 | 65 | 1.42 |
| MGC29898 | | 133015 | — | 119 | 84 | 1.42 |
| TTC9 | KIAA0227 | 23508 | — | 285 | 200 | 1.42 |
| GGCX | VKCFD1 | 2677 | Catalytic | 68 | 48 | 1.42 |
| STC2 | STCRP | 8614 | Signal transduction | 65 | 45 | 1.42 |
| FADS3 | CYB5RP | 3995 | Catalytic | 176 | 124 | 1.42 |

TABLE 3
Sequences from HG-U133B up regulated in the ventral tegmentum

| UniGene | Gene | VT | DT | Change |
|---|---|---|---|---|
| Hs.130544* | UNKNOWN | 85 | 32 | 2.62 |
| Hs.370410* | KIAA1145 | 316 | 126 | 2.50 |
| Hs.9887* | UNKNOWN | 174 | 76 | 2.28 |
| Hs.27261* | UNKNOWN | 232 | 115 | 2.01 |
| Hs.132591* | SLC10A4 | 178 | 92 | 1.93 |
| Hs.30875* | LOC284033 | 293 | 169 | 1.74 |
| Hs.524341* | SLC2A13 | 224 | 130 | 1.72 |
| Hs.479853 | EPHA5 | 268 | 156 | 1.72 |
| Hs.21374* | UNKNOWN | 182 | 106 | 1.71 |
| Hs.464848* | B4GALT6 | 155 | 92 | 1.69 |
| Hs.370410* | KIAA1145 | 226 | 134 | 1.68 |
| Hs.517868* | LRP15/LRRC3B | 203 | 125 | 1.62 |
| Hs.302130* | ELOVL3/CIG30 | 97 | 61 | 1.59 |
| Hs.7413 | UNKNOWN | 207 | 132 | 1.58 |
| Hs.26608* | C20orf100 | 607 | 391 | 1.56 |
| Hs.477370 | DAB1 | 129 | 83 | 1.56 |
| Hs.514946 | CBLN2 | 480 | 311 | 1.55 |
| Hs.143434 | CNTN1 | 406 | 265 | 1.54 |
| Hs.388749 | KGNIP4 | 325 | 215 | 1.51 |
| Hs.486376 | TCBA1 | 231 | 156 | 1.47 |
| Hs.211236 | NTNG1 | 284 | 192 | 1.47 |
| Hs.181788 | UNKNOWN | 67 | 46 | 1.45 |
| Hs.444412 | UNKNOWN | 138 | 95 | 1.44 |
| Hs.275757 | DDR2 | 115 | 80 | 1.43 |
| Hs.275757 | DDR2 | 104 | 73 | 1.43 |
| Hs.470544 | PPIG | 67 | 47 | 1.43 |
| Hs.22247 | UNKNOWN | 148 | 103 | 1.43 |
| Hs.207457 | UNKNOWN | 135 | 94 | 1.42 |

TABLE 4
Reference to sequences

| | | | |
|---|---|---|---|
| Gene | Gene ID | UniGene ID | RefSeq Transcript ID |

Transmembrane

| | | | |
|---|---|---|---|
| KIAA1145 | 57458 | 370410 | NM_020698 |
| SLC10A4 | 132591 | 201780 | NM_152679 |
| SLC2A13 | 114134 | 524341 | NM_052885 |
| LRRC3B | 116135 | 517868 | NM_052953 |
| CBLN2 | 147381 | 514946 | NM_182511 |
| CNTN1 | 1272 | 549027 | NM_001843 |
| | | | NM_175038 |
| NTNG1 | 22854 | 211236 | NM_014917 |
| DDR2 | 4921 | 275757 | NM_001014796 |
| | | | NM_006182 |
| GABRB1 | 2560 | 27283 | NM_000812 |
| SCARB2 | 950 | 349656 | NM_005506 |
| FLJ11627 | — | 137567 | — |
| CORIN | 10699 | 518618 | NM_006587 |
| DLK1 | 8788 | 533717 | NM_003836 |
| PCDH11X | 83259 | 546448 | NM_032971 |
| | | | NM_032972 |
| | | | NM_032973 |
| GRM8 | 2918 | 449625 | NM_000845 |
| SLC17A6 | 57084 | 242821 | NM_020346 |
| CLDN11 | 5010 | 31595 | NM_005602 |
| IL-17RC | 84818 | 129959 | NM_153460 |
| | | | NM_153461 |
| | | | NM_153462 |
| | | | NM_153463 |
| | | | NM_153464 |
| | | | NM_032732 |
| SDC1 | 6382 | 224607 | NM_001006946 |
| | | | NM_002997 |
| ADRA1B | 147 | 368632 | NM_000679 |
| OR12D2 | 26529 | 247862 | NM_013936 |
| GRB10 | 2887 | 164060 | NM_001001549 |
| | | | NM_001001550 |
| | | | NM_001001555 |
| | | | NM_005311 |
| FPRL1 | 2358 | 99855 | NM_001005738 |
| | | | NM_001462 |
| ADCY2 | 108 | 481545 | NM_020546 |
| CD14 | 929 | 163867 | NM_000591 |
| TA-LRRP | 23507 | 482017 | NM_015350 |
| SV2B | 9899 | 8071 | NM_014848 |
| TRPM3 | 80036 | 47288 | NM_001007470 |
| | | | NM_001007471 |
| | | | NM_020952 |
| | | | NM_024971 |
| | | | NM_206944 |
| | | | NM_206945 |
| | | | NM_206946 |
| | | | NM_206947 |
| | | | NM_206948 |
| LRRTM4 | 80059 | 285782 | NM_024993 |
| SPAG4 | 6676 | 123159 | NM_003116 |
| PAQR6 | 79957 | 235873 | NM_024897 |
| | | | NM_198406 |

Transcription

| | | | |
|---|---|---|---|
| FLJ45455 | 388336 | 441035 | NM_207386.1 |
| C20orf100 | 84969 | 26608 | NM_032883 |
| FLJ21924 | 79832 | 369368 | NM_024774 |
| COBLL1 | 22837 | 470457 | NM_014900 |
| DSCR1L1 | 10231 | 440168 | NM_005822 |
| TRIM9 | 114088 | 368928 | NM_015163 |
| | | | NM_052978 |

TABLE 5
Growth factors expressed in 8w GA midbrain.

| Gene | AFFY_ID | Average | Unigene ID | IPI ID | REFSEQ ID |
|---|---|---|---|---|---|
| FGF13 | 205110_s_at | 841 | Hs.6540 | IPI00066574.1 | NP_378668 |
| CSPG5 | 39966_at | 839 | Hs.45127 | IPI00008586.2 | NP_006565 |
| HDGF | 200896_x_at | 783 | Hs.506748 | IPI00020956.1 | NP_004485 |
| LASS1 | 206397_x_at | 506 | Hs.412355 | IPI00019442.1 | NP_001483 |
| IGF1 | 209542_x_at | 487 | Hs.160562 | IPI00001610.1 | NP_000609 |
| RABEP1 | 225064_at | 397 | Hs.551518 | IPI00293009.3 | NP_004694 |
| JAG1 | 209099_x_at | 390 | Hs.224012 | IPI00099650.2 | NP_000205 |
| FGF9 | 206404_at | 360 | Hs.111 | IPI00011172.1 | NP_002001 |
| BMP2 | 205289_at | 314 | Hs.73853 | IPI00025069.1 | NP_001191 |
| BMP15 | 221332_at | 310 | Hs.532692 | IPI00001485.2 | NP_005439 |
| FGF6 | 208417_at | 283 | Hs.166015 | IPI00022396.1 | NP_066276 |
| GDF3 | 220053_at | 272 | Hs.86232 | IPI00299659.6 | NP_065685 |
| PDGFB | 204200_s_at | 255 | Hs.1976 | IPI00000044.1 | NP_002599 |
| KITLG | 226534_at | 220 | Hs.1048 | IPI00220142.1 | NP_003985 |
| TFF1 | 205009_at | 212 | Hs.162807 | IPI00022283.1 | NP_003216 |
| BMP1 | 202701_at | 211 | Hs.1274 | IPI00218044.1 | NP_006121 |
| INHBB | 205258_at | 197 | Hs.1735 | IPI00297026.5 | NP_002184 |
| GDF5 | 206614_at | 177 | Hs.1573 | IPI00015522.2 | NP_000548 |
| HBEGF | 203821_at | 166 | Hs.799 | IPI00012948.2 | NP_001936 |
| TGFB1 | 203084_at | 160 | Hs.1103 | IPI00000075.1 | NP_000651 |
| FGF2 | 230329_s_at | 154 | Hs.284244 | IPI00154603.2 | NP_001997 |
| NODAL | 230916_at | 154 | Hs.370414 | IPI00045497.1 | NP_060525 |
| VWF | 202112_at | 152 | Hs.440848 | IPI00023014.1 | NP_000543 |

TABLE 6
Predicted growth factors expressed in 8w GA midbrain.

| Gene | Affy_ID | AVR | ProtFun | UniGene | IPI | RefSeq |
|---|---|---|---|---|---|---|
| TNFRSF25 | 210847_x_at | 173 | 10.234 | Hs.462529 | IPI00219573.1 | NP_683872 |
| SLC25A29 | 231664_at | 385 | 9.582 | Hs.497598 | IPI00414250.1 | NP_689546 |
| MGC40499 | 227313_at | 167 | 9.410 | Hs.369867 | IPI00102962.2 | NP_689968 |
| NRN1 | 218625_at | 596 | 8.841 | Hs.103291 | IPI00470625.1 | NP_057672 |
| FLJ20519 | 221222_s_at | 248 | 7.821 | Hs.549171 | IPI00550533.2 | NP_060330 |
| FLJ20519 | 229120_s_at | 225 | 7.821 | Hs.549171 | IPI00550533.2 | NP_060330 |
| MGC61716 | 214364_at | 179 | 6.864 | Hs.159556 | IPI00376346.3 | NP_872307 |
| MGC61716 | 226486_at | 171 | 6.864 | Hs.159556 | IPI00376346.3 | NP_872307 |
| LOC387758 | 226769_at | 194 | 6.689 | Hs.32478 | IPI00152072.1 | NP_976249 |
| SPOCK3 | 206433_s_at | 151 | 6.128 | Hs.481133 | IPI00555693.1 | NP_058646 |
| SPOCK3 | 235342_at | 251 | 6.128 | Hs.481133 | IPI00555693.1 | NP_058646 |
| DKFZP564K1964 | 223170_at | 355 | 6.071 | Hs.3447 | IPI00023540.1 | NP_056359 |
| MGC21688 | 221904_at | 247 | 5.802 | Hs.436847 | IPI00412618.1 | NP_653236 |
| GRCA | 205056_s_at | 156 | 5.295 | Hs.418105 | IPI00004405.1 | NP_055264 |
| EGFL9 | 220262_s_at | 234 | 5.007 | Hs.337251 | IPI00028381.2 | NP_076421 |
| OS-9 | 200714_x_at | 805 | 4.975 | Hs.527861 | IPI00186581.6 | NP_001017956 |
| FLJ10803 | 209445_x_at | 208 | 4.042 | Hs.289007 | IPI00018966.4 | NP_060694 |
| C14orf112 | 223191_at | 431 | 3.871 | Hs.137108 | IPI00009365.3 | NP_057552 |
| FAM19A2 | 241399_at | 266 | 3.722 | Hs.269745 | IPI00166553.2 | NP_848634 |
| MGC34647 | 237046_x_at | 167 | 3.667 | Hs.461214 | IPI00167031.2 | NP_689669 |
| C1QTNF4 | 223708_at | 528 | 3.663 | Hs.119302 | IPI00011094.1 | NP_114115 |
| WNT16 | 221113_s_at | 204 | 3.376 | Hs.272375 | IPI00002791.1 | NP_476509 |
| WNT16 | 224022_x_at | 239 | 3.376 | Hs.272375 | IPI00002791.1 | NP_476509 |
| CCNB1IP1 | 217988_at | 213 | 2.716 | Hs.107003 | IPI00375303.1 | NP_067001 |
| KITLG | 226534_at | 220 | 2.489 | Hs.1048 | IPI00009450.1 | NP_000890 |
| WNT7B | 217681_at | 204 | 2.399 | Hs.512714 | IPI00011031.2 | NP_478679 |

1. A human embryonal stem cell, a human neural stem cell, a human neural precursor cell, a human neural cell, or a human dopaminergic neuron being genetically modified to overexpress at least one gene selected from the group consisting of genes from Table 2, that are not marked with bold, and genes from Table 3, 4, and 6.

2. The cell of claim 1, being isolated from the human body.

3. (canceled)

4. The cell of claim 1, wherein the gene has the GO annotation “signal transduction” or “binding” in Table 2.

5. The cell of claim 1, wherein the gene is selected from the group consisting of genes from Table 4 and 6.

6. The cell of claim 1, wherein the gene is selected from the group consisting of transmembrane genes from Table 4.

7. The cell of claim 6, wherein the gene is selected from the group consisting of KIAA1145, SLC10A4, SLC2A13, and LRRC3B.

8. The cell of claim 1, wherein the gene is selected from the group consisting of transcription factor genes from Table 4.

9. The cell of claim 8, wherein the gene is selected from the group consisting of FLJ45455 and C200rf100.

10. The cell of claim 1, wherein the gene is selected from the group consisting of genes from Table 6.

11. The cell of claim 10, wherein the gene is selected from the group consisting of TNFRSF25, SLC25A29, MGC40499, NRN1, FLJ20519, FLJ20519, MGC61716, MGC61716, LOC387758, SPOCK3, SPOCK3, DKFZP564K1964, MGC21688, GRCA, and EGFL9.

12. The cell of claim 11, wherein the gene is selected from the group consisting of TNFRSF25, SLC25A29, MGC40499, NRN1, FLJ20519, and FLJ20519.

13. The cell of claim 10, wherein the gene is selected from the group consisting of OS-9, NRN1, C1QTNF4, C14orf112, SLC25A29, DKFZP564K1964, FAM19A2, and SPOCK3.

14. The cell of claim 13, wherein the gene is selected from the group consisting of OS-9, NRN1, ClQTNF4, and C14 or f112.

15. The cell of claim 10, wherein the gene encodes a mature part of said protein.

16. A method for enhancing the generation of dopaminergic neurons, comprising administering to a human cell at least one protein encoded by a gene selected from the group consiting of genes from Table 2, that are not marked with bold, and genes from Table 3, 4 , 5 and 6.

17. The method of claim 16, wherein the human cell is selected from the group consisting of human embryonal stem cells, human neural stem cells, human neural precursor cells, human neurons, and human dopaminergic neurons.

18. The method of claim 16, wherein the gene encodes a transcription factor.

19. The method of claim 16, wherein the gene encodes a protein involved in signal transduction.

20. The method of claim 16, wherein said protein is administered as a protein formulation.

21. The method of claim 20, wherein the formulation comprises the mature part of said protein.

22. The method of claim 16, wherein the gene is selected from the group of genes from Table 6.

23. The method of claim 22, wherein the gene is selected from the group consisting of TNFRSF25, SLC25A29, MGC40499, NRN1, FLJ20519, FLJ20519, MGC61716, MGC61716, LOC387758, SPOCK3, SPOCK3, DKFZP564K1964, MGC21688, GRCA, and EGFL9.

24. The method of claim 23, wherein the gene is selected from the group consisting of TNFRSF25, SLC25A29, MGC40499, NRN1, FLJ20519, and FLJ20519.

25. The method of claim 22, wherein the gene is selectred from the group consisting of OS-9, NRN1, C1QTNF4, C14orf112, SLC25A29, DKFZP564K1964, FAM19A2, and SPOCK3.

26. The method of claim 25, wherein the gene is selected from the group consisting of OS-9, NRN1, ClQTNF4, and CF14orf112.

27. The method of claim 16, wherein the gene is selected from the group of genes from Table 5.

28. The method of claim 27, wherein the gene is selected group consisting of FGF13, CSPG5, HDGF, LASS1, IGF1, RABEP1JAG1, FGF9, BMP2, BMP15, FGF6, GDF3, and PDGFB.

29. The method of of claim 28, wherein is selected from the group consisting of FGF13, CSPG5, SS1, and IGF1.

30. The method of claim 16, wherein the protein is administered by causing said gene to be overexpressed in said cell.

31. The method of claim 30, wherein said overexpression is caused by transducing or transfecting said cell with an expression vector coding for said gene.

32. The method of claim 31, wherein the transduction/transfection is performed in vitro.

33. The method of claim 31, wherein the transduction/transfection is performed in vivo.

34. The method of claim 31, wherein the vector is a virus vector.

35. The method of claim 31, wherein the cell is transfected using lipofection, electroporation, or calcium phospate transfection.

36. The method of claim 16, further comprising the use of a standard dopaminergic differentiation protocol.

37. A method for enriching a population of cells comprising dopaminergic neurons or dopaminergic precursor neurons, said method comprising labelling the cells with a label specific for the expression of at least one gene selected from the group consisting of genes from Table 2, that are not marked with bold, and genes from Table 3, 4 , 5 and 6 and sorting the cells.

38. The method of claim 37, wherein the label comprises an antibody.

39. The method of claim 37, wherein the label comprises a nucleotide probe.

40. The method of claim 37, wherein the gene is selected from genes from Table 4.

41. The method of claim 40, wherein the gene is annotated in Table 4 as a transmembrane gene.

42. The method of claim 41, wherein the gene is selectred from the group consisting of KIAA1145, SLC10A4, SLC2A13, and LRRC3B.

43. A method of treatment of Parkinson's disease, said method comprising administering to a patient in need thereof a therapeutically effective amount of at least one protein encoded by a gene selected from the group consisting of genes from Table 2, that are not marked with bold, and genes from Table 3, 4, 5 and 6.

44. The method of claim 43, wherein the gene is selected from the group consisting of genes from Table 3, 4 and 6.

45. The method of claim 44, wherein the gene is annotated in Table 4 as a transmembrane gene.

46. The method of claim 45, wherein the gene is selected from the group consisting of KIAA1145, SLC10A4, SLC2A13, 3B.

47. The method of claim 44, wherein the gene is annotated in Table 4 as a transcription factor.

48. The method of claim 47, wherein the gene is FLJ45455 and C200rf100.

49. The method of claim 44, wherein the gene is selected from the group of genes from Table 6.

50. The method of claim 49, wherein the gene is selected from the group consisting of TNFRSF25, SLC25A29, MGC40499, NRN1, FLJ20519, FLJ20519, MGC61716, MGC61716, LOC387758, SPOCK3, SPOCK3, DKFZP564K1964, MGC21688, GRCA, and EGFL9.

51. The method of claim 50, wherein the gene is selected from the group consisting of TNFRSF25, SLC25A29, MGC40499, NRN1, FLJ20519, and FLJ20519.

52. The method of claim 49, wherein the gene is selected from the group consisting of OS-9, NRN1, C1QTNF4, C14orf112, SLC25A29, DKFZP564K1964, FAM19A2, and SPOCK3.

53. The method of claim 52, wherein the gene is selected from the group consisting of OS-9, NRN1, ClQTNF4, and C14orf112.

54. The method of claim 44, wherein the gene is selected from the group of genes from Table 5.

55. The method of claim 54, wherein the gene is selected from the group consisting of FGF13, CSPG5, HDGF, LASS1, IGF1, RABEP1, JAG1, FGF9, BMP2, BMP15, FGF6, GDF3, and PDGFB.

56. The method fo of claim 55, wherein is selected from the group consisting of FGF13, CSPG5, HDGF, LASS1, and IGF1.

57. The method of claim 43, wherein said protein is administered as a protein formulation.

58. The method of claim 43, wherein the gene encodes a growth factor, and said growth factor is administered by implanting a composition of cells secreting said protein in the striatum or substantia nigra of a subject.

59. The method of claim 58, wherein the cells are encapuslated behind a semipermeable immunoisolatory membrane.

60. The method of claim 43, wherein the protein is administered by causing said gene to be overexpressed in said cell.

61. The method of claim 60, wherein said overexpression is caused by transducing or transfecting said cell with an expression vector coding for said gene.

62. The method of claim 61, wherein the transduction/transfection is performed in vivo.

63. The method of claim 61, wherein the vector is a virus vector.
